(19) United States
(12) Patent Application Publication
(10) Pub. No.: US 2022/0071932 A1
(43) Pub. Date: Mar. 10, 2022

(54) Compositions and Methods Against Stress-Induced Affective Disorders and Their Associated Symptoms

(71) Applicants: The Trustees of Columbia University in the City of New York, New York, NY (US); The Research Foundation for Mental Hygiene, Inc., Menands, NY (US)

(72) Inventors: Christine A. DENNY, Ho-Ho-Kus, NJ (US); Briana K. CHEN, New York, NY (US)

(73) Assignees: The Trustees of Columbia University in the City of New York, New York, NY (US); The Research Foundation for Mental Hygiene, Inc., Menands, NY (US)

(21) Appl. No.: 17/526,716

(22) Filed: Nov. 15, 2021

Related U.S. Application Data
(63) Continuation of application No. PCT/US2020/032886, filed on May 14, 2020.
(60) Provisional application No. 62/861,765, filed on Jun. 14, 2019.
(60) Provisional application No. 62/848,406, filed on May 15, 2019.

(51) Int. Cl. A61K 31/14; A61K 45/06; A61P 25/22

(52) U.S. Cl. CPC A61K 31/14; A61P 25/22; A61K 45/06

(57) ABSTRACT

Methods for prophylactically treating or treating a stress-induced affective disorder or stress-induced psychopathology in a subject are provided. In certain embodiments, an effective amount of an antagonist of the N-methyl-D-aspartate (NMDA) receptor, such as fluoroethylnormemantine, or a pharmaceutically acceptable salt or derivative thereof, is administered to a subject prior to, during, and/or after, a stressor.

### CROSS-REFERENCE TO RELATED APPLICATIONS

The present application claims priority to U.S. Provisional Patent Application No. 62/848,406 filed on May 15, 2019, and U.S. Provisional Patent Application No. 62/861,765 filed on Jun. 14, 2019, each of which is incorporated herein by reference in its entirety.

### FIELD OF THE INVENTION

The present invention relates to fluoroethylnormemantine (FENM or FNM) compositions and their use in methods of treatment or prevention of stress-induced affective disorders. In certain aspects, a fluoroethylnormemantine composition can be administered prior to, during, or after a stressor.

### BACKGROUND OF THE INVENTION

Major depressive disorder (MDD) is the leading cause of disability worldwide, affecting more than 17 million adults in the US alone (1, 2). The current paradigm for treating MDD is to wait for the illness to develop and then relieve symptoms through behavioral therapy and/or antidepressant treatment. However, this approach is ineffective in up to 40% of patients diagnosed with treatment-resistant depression (TRD) (3). Moreover, the delayed onset of efficacy (between 6-12 weeks) of many commonly-used antidepressants, such as selective serotonin reuptake inhibitors (SSRIs) and monoamine oxidase inhibitors, is a major cause of concern for patients at an increased risk of suicide (4, 5). These significant limitations have led to a search for more efficacious compounds that can prevent and/or treat MDD with a rapid onset of action.

Traditionally, affective disorders have been treated from a symptom-suppression approach. Existing drugs aim to mitigate the impact of these chronic diseases, but do not cure or prevent the disease itself. There are no known cures. Antidepressants are typically used to treat existing depressive symptoms, but chronic antidepressant treatment may also protect against subsequent depressive episodes. This is known as “tertiary prevention” or “tertiary prophylaxis.” Tertiary preventions suppress symptoms and mitigate the impact of a chronic disease, but do not treat the underlying disease. Maintenance treatment in MDD patients is often referred to as prophylaxis against the development of additional depressive episodes, but this should not be confused with “primary prevention” or “primary prophylaxis,” which aims to prevent the disease before it ever occurs. No antidepressants have yet been shown to be primary prophylactics. Whether these tertiary prophylactic antidepressants, which can decrease the incidence of symptomatic episodes in disordered individuals, are also able to work as primary prophylaxis and prevent de novo psychiatric disorders remains to be tested. Importantly, classical antidepressant efficacy is neither sufficient for, nor necessarily predictive of, primary prophylactic efficacy.

Post-traumatic stress disorder (PTSD) is an illness characterized by persistent, vivid re-experiencing of a traumatic event, hyperarousal, and avoidance of stimuli associated with the trauma (Charney et al. (1993): Psychobiologic mechanisms of posttraumatic stress disorder. Arch Gen Psychiatry 50:295-305). PTSD is often comorbid with other prevalent psychiatric illnesses such as major depressive disorder (MDD) (28%) and substance use (73%) (Brady et al., (2000): Comorbidity of psychiatric disorders and posttraumatic stress disorder. J Clin Psychiatry 61: 22-32.). Currently, clinicians rely on several methods to reduce the symptomology of PTSD including pharmacology, psychotherapy, or a combination of both methods. However, given the lack of empirical evidence and clinical utility of psychotherapy for trauma victims, consistent therapies have not been established (National Center for PTSD, 2016).

Previous studies have shown that (R,S)-ketamine, a commonly-used anesthetic, is a rapid-acting antidepressant (6). At a subanesthetic dose, (R,S)-ketamine can relieve depressive symptoms as quickly as 2 hours after administration, lasts up to 3 weeks, and is effective even in patients suffering from TRD (7-10). When administered prior to stress, (R,S)-ketamine can enhance stress resilience to prevent the onset of stress-induced depressive-like behavior as well as attenuate learned fear (11-15). (R,S)-ketamine acts as a noncompetitive glutamate NMDAR antagonist and binds to the open channel pore (16-18). Additional evidence suggests that (R,S)-ketamine activates α-amino-3-hydroxy-5-methyl-4-isoxazolepropionic acid receptors (AMPARs), increases brain-derived neurotrophic factor (BDNF) signaling, and leads to rapid and sustained changes in synaptic plasticity (19-24). However, despite (R,S)-ketamine's remarkable actions, the compound's side effects can include psychotropic effects and high abuse potential, representing a major hurdle towards its development as a clinical treatment. These challenges have led researchers to leverage current knowledge of the mechanistic actions of (R,S)-ketamine in order to find or develop alternative compounds with similar antidepressant/prophylactic efficacy and reduced nonspecific effects.

While some novel compounds, such as stereospecific (R,S)-ketamine metabolites or alternative NMDAR antagonists (e.g., rapastinel, MK-801), are effective in reducing or preventing depressive-like behavior, not all NMDAR antagonists exhibit antidepressant or prophylactic efficacy (20, 25-29). For instance, memantine, a noncompetitive open channel NMDAR antagonist, does not reduce depressive symptoms in patients diagnosed with MDD, but is commonly used to treat moderate to severe confusion (dementia) related to Alzheimer's disease (AD) (30-32). While memantine does not cure AD, it may improve memory, awareness, and the ability to perform daily functions. To date, memantine has not been studied as a prophylactic compound. Thus, although memantine lacks specific antidepressant efficacy, it does have pro-cognitive actions.

The present disclosure provides for novel NMDAR antagonists that have antidepressant and/or prophylactic efficacy as well as pro-cognitive effects.

### SUMMARY

The present disclosure provides for a method for preventing or delaying a stress-induced affective disorder or stress-induced psychopathology in a subject. The method may comprise administering an effective amount of a pharmaceutic composition comprising fluoroethylnormemantine (FENM), or an analog, a pharmaceutically acceptable salt, a derivative, or a metabolite thereof, to the subject at least about 72 hours, about 72 hours to about 3 weeks, about 72 hours to about 2 weeks, about 1 week, at least or about 48 hours, at least or about 72 hours, at least or about 4 days, at least or about 5 days, at least or about 6 days, at least or about 1 week, at least or about 2 weeks, at least or about 3 weeks, or at least or about 4 weeks, prior to a stressor.

The present disclosure provides for a method for treating a stress-induced affective disorder or stress-induced psychopathology in a subject. The method may comprise administering an effective amount of a pharmaceutic composition comprising fluoroethylnormemantine (FENM), or an analog, a pharmaceutically acceptable salt, a derivative, or a metabolite thereof, to the subject within about 12 hours, within about 10 hours, within about 8 hours, within about 6 hours, within about 5 hours, within about 4 hours, within about 3 hours, within about 2 hours, within about 1 hour, within about 45 minutes, within about 30 minutes, within about 20 minutes, within about 15 minutes, within about 10 minutes, or within about 5 minutes, after a stressor.

The pharmaceutic composition may be administered to the subject once, twice, three times, four times or more prior to, during, or after, a stressor.

The pharmaceutic composition may be administered orally, intravenously, intranasally, or via injection to the subject.

The stress-induced affective disorder may comprise depression and/or fear. The stress-induced affective disorder may comprise major depressive disorder and/or posttraumatic stress disorder (PTSD). The stress-induced affective disorder may be selected from the group consisting of: depressive-like behavior and associated affective disorders, anhedonic behavior and associated affective disorders, anxiety and associated affective disorders, cognitive impairments and deficits and associated disorders, and combinations thereof. The stress-induced affective disorder may comprise stress-induced psychopathology, such as depressive behavior.

The present method may prevent or delay stress-induced cognitive impairment and/or decline.

The present method may further comprise administering an effective amount of an anti-depressant, an anxiolytic, or combinations thereof.

The present method may further comprise administering an effective amount of a selective serotonin reuptake inhibitor (SSRI), or a pharmaceutically acceptable salt or derivative thereof.

The present method may further comprise administering an effective amount of fluoxetine, paroxetine, sertraline, lithium, riluzole, prazosin, lamotrigine, ifenprodil, or combinations thereof.

The subject may be a mammal, such as a human.

The present disclosure provides for a method of preventing or treating the onset of anxiety in a subject. The method may comprise administering an effective amount of a pharmaceutic composition comprising fluoroethylnormemantine (FENM), or an analog, a pharmaceutically acceptable salt, a derivative, or a metabolite thereof, to the subject, wherein the subject exhibits clinical signs of anxiety.

Also encompassed by the present disclosure is a method of preventing or treating a recurrence of depression in a subject. The method may comprise administering an effective amount of a pharmaceutic composition comprising fluoroethylnormemantine (FENM), or an analog, a pharmaceutically acceptable salt, a derivative, or a metabolite thereof, to the subject, wherein the subject has recovered from at least one depressive episode.

The present disclosure provides for a method of treating depression in a subject. The method may comprise administering an effective amount of a pharmaceutic composition comprising fluoroethylnormemantine (FENM), or an analog, a pharmaceutically acceptable salt, a derivative, or a metabolite thereof, to the subject who is depressed.

One or more methods are described herein for preventing or treating stress-induced depression in a mammal by administering a therapeutically effective amount of fluoroethylnormemantine (FENM) to the mammal. Furthermore, one or more methods are described for preventing the onset of anxiety or fear in a mammal by administering a therapeutically effective amount of FENM to the mammal.

The present invention provides for a method of treating fear or depression in a mammal, comprising administering a therapeutically effect amount of fluoroethylnormemantine (FENM) at least about one week before the mammal is subjected to at least one stress event.

The present invention also provides for a method of treating fear or depression in a mammal, comprising administering a therapeutically effect amount of FENM within about 15 minutes after the mammal is subjected to at least one stress event. In one embodiment, FENM is administered within about 30 minutes after the mammal is subjected to at least one stress event. In another embodiment, FENM is administered within about 45 minutes after the mammal is subjected to at least one stress event. In another embodiment, FENM is administered within about 1 hour after the mammal is subjected to at least one stress event.

The present invention also provides for a method of treating fear, anxiety, or depression in a mammal, comprising administering a therapeutically effective amount of FENM within about one hour after the mammal is subjected to at least one stress event.

The present invention also provides for a method of treating depression in a mammal, comprising administering a therapeutically effective amount of FENM to the mammal who has exhibited or is exhibiting clinical signs of depression.

The present invention also provides for a method of treating the onset of anxiety in a mammal, comprising administering a therapeutically effective amount of FENM to the mammal, wherein the mammal is exhibiting clinical signs of anxiety.

The present invention also provides for a method of treating a recurrence of depression in a mammal, comprising administering a therapeutically effective amount of FENM to the mammal, wherein the mammal has recovered from at least one depressive episode.

The present invention also provides for a method for treating a stress-associated disorder comprising administering to a mammal at risk of having a stress-associated disorder FENM prior to, during, or within about one hour following a stress-related event in an effective amount to prevent the stress-associated disorder. In certain embodiments, the stress-associated disorder is post-traumatic stress disorder (PTSD). In certain embodiments, FENM is administered orally. In certain embodiments, FENM is administered daily beginning one week prior to the stress-related event. In certain embodiments, FENM is administered throughout the duration of the stress-related event. In certain embodiments, FENM is administered for up to 24 weeks after the stress-related event.

The present invention provides for methods of preventing and/or treating stress-induced depression and related psychiatric diseases. A subject exhibiting clinical signs of depression is administered FENM either before or after a stressful event, as a prophylactic and/or antidepressant. Relevant psychiatric diseases include depression, anxiety, and PTSD. Furthermore, one or more methods are described for preventing pathological behavior induced by chronic and/or acute stress in a mammal by administering a therapeutically effective amount of FENM to the mammal.

### BRIEF DESCRIPTION OF THE DRAWINGS

FIGS. 1A-1P. FENM attenuates learned fear and protects against stress-induced depressive-like behavior when administered 1 week prior to CFC. (A) Experimental protocol. (B) Freezing was comparable between all groups during CFC training. (C-D) (R,S)-ketamine (30 mg/kg) and FENM (20 and 30 mg/kg) significantly decreased fear expression. (E) On day 1 of the FST 1, all groups exhibited comparable immobility time. (F-G) On day 2 of the FST, all drugs and doses tested significantly reduced immobility time when compared with saline controls. (H-J) In the OF, (R,S)-ketamine and FENM did not alter distance travelled or time spent in the center of the arena. (K) In the MB task, all groups buried a comparable number of marbles. (L-M) (R,S)-ketamine and FENM did not alter distance travelled or time spent in the open arms and center of the EPM. (N) (R,S)-ketamine and FENM did not alter latency to feed in the NSF. (O-P) Body weight loss and food eaten in the NSF were comparable between all drug groups. (n=5-12 male mice per group). Error bars represent ±SEM. *p<0.05, **p<0.01, ***p<0.001. Sal, saline; K, (R,S)-ketamine; FENM, fluoroethylnormemantine; CFC, contextual fear conditioning; FST, forced swim test; OF, open field; MB, marble burying; EPM, elevated plus maze; NSF, novelty suppressed feeding; sec, second; min, minute; mg, milligram; kg, kilogram; cm, centimeter; m, meter; no., number; g, gram.

FIGS. 2A-2P. FENM is a novel antidepressant when administered following stress. (A) Experimental design. (B) Freezing was comparable between all groups during CFC training. (C-D) During CFC re-exposure, freezing was comparable between all groups. (E) On day 1 of the FST 1, all groups exhibited comparable immobility time. (F-G) On day 2 of the FST, all drugs and doses tested significantly reduced immobility time when compared with saline mice. (H-J) In the OF, all groups of mice travelled a comparable distance travelled and spent a comparable amount of time in the center of the arena. (K) In the MB task, FENM (20 and 30 mg/kg) decreased the number of marbles buried when compared with saline. (L-M) In the EPM, (R,S)-ketamine and FENM did not alter distance travelled or the time spent in the open arms and center of the maze. (N) In the NSF, (R,S)-ketamine and FENM did not alter the latency to feed in the open arena. (O-P) Body weight loss and food eaten in the home cage were comparable between all drug groups. (n=6 male mice per group). Error bars represent ±SEM. *p<0.05, **p<0.01, ***p<0.001. Sal, saline; K, (R,S)-ketamine; FENM, fluoroethylnormemantine; CFC, contextual fear conditioning; FST, forced swim test; OF, open field; MB, marble burying; EPM, elevated plus maze; NSF, novelty suppressed feeding; sec, second; min, minute; mg, milligram; kg, kilogram; cm, centimeter; m, meter; no., number; g, gram.

FIGS. 3A-3S. FENM is also an effective antidepressant following a shorter intertrial interval between stress and depression assays. (A) Experimental design. (B-C) Freezing was comparable between all groups during CFC training. (D-G) During CFC re-exposures 1 and 2, all groups of mice exhibited comparable freezing. (H-I) On day 1 of the FST 1, all groups exhibited comparable immobility time. (J-K) On day 2 of the FST, FENM, but not (R,S)-ketamine significantly reduced immobility time when compared with saline mice. (L-M) In the OF, all groups of mice travelled a comparable distance travelled and spent a comparable amount of time in the center of the arena. (N) In the MB task, all groups of mice buried a comparable number of marbles. (0) In the EPM, all groups spent a comparable amount of time in the open arms and center of the maze (P-Q) In the NSF, (R,S)-ketamine and FENM did not alter the latency to feed in the open arena or the homecage. (R-S) Body weight loss and food eaten in the home cage were comparable between all groups. (n=6 male mice per group). Error bars represent ±SEM. *p<0.05, **p<0.01, ***p<0.001. Sal, saline; K, (R,S)-ketamine; FENM, fluoroethylnormemantine; CFC, contextual fear conditioning; FST, forced swim test; OF, open field; MB, marble burying; EPM, elevated plus maze; NSF, novelty suppressed feeding; sec, second; min, minute; mg, milligram; kg, kilogram; cm, centimeter; m, meter; no., number; g, gram.

FIGS. 4A-4S. FENM attenuates fear when administered following an extinction trial. (A) Saline, (R,S)-ketamine (30 mg/kg), or FENM (20 mg/kg) was administered 5 min following re-exposure 1 (24 h before re-exposure 2). During CFC training (B-C) and re-exposure 1 (D-E), freezing was comparable between all groups. (F-G) During re-exposure 2, FENM, but not (R,S)-ketamine, significantly decreased fear expression when compared with saline. During day 1 (H-I) and day 2 (J-K) of the FST, all groups exhibited a comparable immobility time. (L) In the OF, all groups traveled a comparable distance. (M) FENM significantly increased time spent in the center when compared with saline. (N) The number of marbles buried was comparable in all groups. (0) In the EPM, the time spent in the open and closed arms was comparable in all groups. In the NSF, FENM and (R,S)-ketamine did not alter the latency to eat in (P) the NSF arena or (Q) the homecage. (R) All groups lost a comparable amount of weight. (S) All groups ate a comparable amount of food. (n=7-11 male mice per group). Error bars represent ±SEM. *p<0.05, **p<0.01, ***p<0.001. Sal, saline; FENM, fluoroethylnormemantine; CFC, contextual fear conditioning; FST, forced swim test; OF, open field; MB, marble burying; EPM, elevated plus maze; NSF, novelty suppressed feeding; cm, centimeters; sec, seconds; min, minutes; g, grams; no, number.

FIGS. 5A-5N. FENM does not alter NMDAR expression. (A) Behavioral protocol. Male mice were given a single administration of saline, (R,S)-ketamine (30 mg/kg), or FENM (20 mg/kg) one week prior to three-shock CFC training. Five days later, mice were re-exposed to the training context. One hour after re-exposure, mice were sacrificed, and brain tissue was collected for use in either immunohistochemistry or Western blot analysis. (B-C) Mice in all groups froze at comparable levels during CFC training. (D-E) Upon re-exposure, (R,S)-ketamine- and FENM-administered mice exhibited significantly less freezing when compared with saline controls. (F) Representative images of c-fos immunohistochemical staining in the (top) dorsal HPC and (bottom) ventral HPC. (G-I) Levels of c-fos⁺ neurons were comparable across all groups in the dDG, dCA3, and dCA1. (J) All groups exhibited comparable levels of c-fos⁺ neurons in the vDG. (K) Mice administered (R,S)-ketamine, but not FENM, had significantly higher numbers of c-fos⁺ neurons in the vCA3. (L) c-fos was comparable across all groups in the vCA1. (M-N) Western blot analysis revealed comparable expression of the NR2A subunit of the NMDAR in the HPC. (n=5-10 mice per group). Error bars represent±SEM. Sal, saline; K, (R,S)-ketamine; FENM, fluoroethylnormemantine; CFC, contextual fear conditioning; sac, sacrifice; min, minute; DG, dentate gyrus; CA3, Cornu Ammonis area 3; CA1, Cornu Ammonis area 1; no., number; dDG, dorsal dentate gyrus; dCA3, dorsal Cornu Ammonis area 3; dCA1, dorsal Cornu Ammonis area 1; vDG, ventral dentate gyrus; vCA3, ventral Cornu Ammonis area 3; vCA1, ventral Cornu Ammonis 1; kDa, kilodalton; β-tub, beta-tubulin.

FIGS. 6A-6E. FENM does not alter immobility time in the FST in non-stressed 129S6/SvEv mice. (A) Experimental protocol. Saline or FENM was administered 1 h before the start of the FST. (B-C) Both groups of mice had comparable immobility during day 1 of the FST. (D-E) Both groups of mice had comparable immobility during day 2 of the FST. (n=5 male mice per group). Error bars represent ±SEM. *p<0.05, **p<0.01, ***p<0.001. Sal, saline; FENM, fluoroethylnormemantine; FST, forced swim test; h, hour; sec, seconds; min, minutes.

FIGS. 7A-7M. FENM does not alter anxiety-like behavior, but decreases hypophagia in non-stressed 129S6/SvEv mice. (A) Experimental protocol. Saline or FENM was administered 1 h before the start of the OF. (B-C) Both groups of mice travelled a comparable distance in the OF. Both groups of mice spent a comparable amount of time in the center (D) and the periphery (E). (F) In the MB paradigm, FENM did not affect the number of marbles buried. In the EPM, both groups of mice spent a comparable amount of time in the (G) open arms, (H) closed arms, and (I) center. (J) In the NSF paradigm, FENM administration significantly decreased the latency to approach the pellet in the NSF arena when compared with saline administration. (K) However, FENM did not alter the latency to eat in the homecage. (L) Both groups of mice ate a comparable amount of food in the home cage. (M) FENM did not impact the amount of weight loss in the NSF paradigm. (n=10 male mice per group). Error bars represent ±SEM. *p<0.05, **p<0.01, ***p<0.001. Sal, saline; FENM, fluoroethylnormemantine; OF, open field; MB, marble burying; EPM, elevated plus maze; NSF, novelty suppressed feeding; cm, centimeters; sec, seconds; min, minutes; g, grams, no, number; h, hour.

FIGS. 8A-8S. FENM is a not prophylactic against stress-induced depressive-like when administered 5 minutes before CFC. (A) Saline, (R,S)-ketamine (30 mg/kg), or FENM (20 mg/kg) was administered 5 min prior to CFC. (B-C) During CFC training, (R,S)-ketamine, but not FENM, significantly decreased freezing when compared with saline. (D-E) During re-exposure 1, (R,S)-ketamine, but not FENM significantly decreased fear expression. (F-G) During re-exposure 2, (R,S)-ketamine significantly decreased fear expression when compared with saline. FENM significantly increased fear expression when compared with saline. (H-I) On day 1 of the FST 1, all groups exhibited comparable immobility time. (J-K) On day 2 of the FST, (R,S)-ketamine, but not FENM, significantly decreased immobility time when compared with saline. (L-M) In the OF, (R,S)-ketamine and FENM did not alter distance travelled or time spent in the center of the arena. (N) In the MB task, all groups buried a similar number of marbles. (0) In the EPM, all groups spent a comparable amount of time in the open arms and center of the maze. (P) In the NSF, (R,S)-ketamine, but not FENM, increased the latency to feed in the open arena when compared with saline. (Q) All groups fed at a comparable latency in the homecage. (R-S) Moreover, body weight loss and food eaten in the homecage were comparable between the groups. (n=6 male mice per group). Error bars represent ±SEM. *p<0.05, **p<0.01, ***p<0.001. Sal, saline; FENM, fluoroethylnormemantine; CFC, contextual fear conditioning; FST, forced swim test; OF, open field; MB, marble burying; EPM, elevated plus maze; NSF, novelty suppressed feeding; cm, centimeters; sec, seconds; min, minutes; g, grams; no, number.

FIGS. 9A-9S. FENM does not attenuate fear when administered prior to an extinction trial. (A) Saline, (R,S)-ketamine (30 mg/kg), or FENM (20 mg/kg) was administered 5 minutes prior to re-exposure 1. During CFC training (B-C), re-exposure 1 (D-E), and re-exposure 2 (F-G), freezing was comparable between all groups. During day 1 (H-I) and day 2 (J-K) of the FST, all groups exhibited comparable immobility time. (L-M) In the OF, all groups travelled a comparable distance and spent a comparable amount of time in the center. (N) The number of marbles buried was comparable in all groups. (0) In the EPM, the time spent in the open and closed arms was comparable in all groups. In the NSF, FENM or (R,S)-ketamine did not alter the latency to eat in (P) the NSF arena or (Q) the homecage. (R) All groups lost a comparable amount of weight. (S) All groups ate a comparable amount of food. (n=6 male mice per group). Error bars represent ±SEM. *p<0.05, **p<0.01, ***p<0.001. Sal, saline; FENM, fluoroethylnormemantine; CFC, contextual fear conditioning; FST, forced swim test; OF, open field; MB, marble burying; EPM, elevated plus maze; NSF, novelty suppressed feeding; cm, centimeters; sec, seconds; min, minutes; g, grams; no, number.

### DETAILED DESCRIPTION OF THE INVENTION

The present disclosure provides methods for prophylaxis and/or treatment of a stress-induced affective disorder or stress-induced psychopathology in a subject, such as depression and/or fear. Also encompassed by the present disclosure are methods for inducing and/or enhancing stress resilience in a subject. In certain embodiments, an effective amount of an antagonist of the N-methyl-D-aspartate receptor (NMDA receptor or NMDAR), such as fluoroethylnormemantine (FENM or FNM) or a pharmaceutically acceptable salt, analog, derivative, or metabolite thereof, is administered to a subject prior to, during, and/or after a stressor.

The present agent/composition may be administered therapeutically to achieve a therapeutic benefit or prophylactically to achieve a prophylactic benefit. By therapeutic benefit is meant eradication or amelioration of the underlying stress-induced affective disorder being treated, and/or eradication or amelioration of one or more of the symptoms associated with the underlying disorder. By prophylactic benefit is meant prevention or delay of the onset of a stress-induced affective disorder, and/or prevention or delay of the onset of one or more of the symptoms associated with a stress-induced affective disorder. In certain embodiments, an effective amount of the present agent/composition to be administered prevents stress-related disorders from developing or being exacerbated into more serious conditions.

In certain embodiments, for prophylactic administration, the present agent/composition may be administered to a patient at risk of developing a stress-induced affective disorder, or to a patient reporting one or more of the physiological symptoms of a stress-induced affective disorder, even though a diagnosis of a stress-induced affective disorder may not have yet been made. In certain embodiments, prophylactic administration is applied to avoid the onset of the physiological symptoms of the underlying disorder, before the symptom manifests cyclically. In this latter embodiment, the therapy is prophylactic with respect to the associated physiological symptoms instead of the underlying indication. In certain embodiments, the present agent/composition is administered prior to recurrence of a stressor. In certain embodiments, the present agent/composition is administered prior to the onset of a particular symptom.

In a further embodiment, the present invention provides for the use of the present agent or a pharmaceutically acceptable salt or solvate thereof, or a physiologically functional derivative or analog thereof, or a metabolite thereof, in the preparation of a medicament for the treatment of a stress-induced affective disorder.

“Treating” or “treatment” of a state, disorder or condition includes: (1) preventing or delaying the appearance of clinical symptoms of the state, disorder, or condition developing in a person who may be afflicted with or predisposed to the state, disorder or condition but does not yet experience or display clinical symptoms of the state, disorder or condition; (2) inhibiting the state, disorder or condition, i.e., arresting, reducing or delaying the development of the disease or a relapse thereof (in case of maintenance treatment) or at least one clinical symptom, sign, or test, thereof; or (3) relieving the disease, i.e., causing regression of the state, disorder or condition or at least one of its clinical or sub-clinical symptoms or signs.

The benefit to a subject to be treated is either statistically significant or at least perceptible to the patient or to the physician.

The present agents include antagonists of the NMDA receptor or NMDAR (also referred to herein as NMDA receptor antagonists or NMDAR antagonists), such as fluoroethylnormemantine (FENM or FNM), pharmaceutically acceptable salts or solvates thereof, analogs thereof, derivatives thereof (e.g., physiologically functional derivatives or analogs thereof), metabolites thereof, and combinations thereof.

A “prophylactically effective amount” refers to an amount effective, at dosages and for periods of time necessary, to achieve the desired prophylactic result. In certain embodiments, since a prophylactic dose is used in subjects prior to or at an earlier stage of a disorder, the prophylactically effective amount is less than the therapeutically effective amount. In certain embodiments, the prophylactically effective amount is similar to, identical to, or more than, the therapeutically effective amount.

A therapeutically effective amount, or an effective amount, of a drug is an amount effective to demonstrate a desired activity of the drug. A “therapeutically effective amount” will vary depending on the compound, the disorder and its severity and the age, weight, physical condition and responsiveness of the subject to be treated. In certain embodiments, an effective amount of an NMDA receptor antagonist such as fluoroethylnormemantine, or a pharmaceutically acceptable salt or solvate thereof, or a derivative or analog thereof, or a metabolite thereof, is an amount effective to prevent or delay the onset of a stress-induced affective disorder, and/or effective to alleviate, one or more of the symptoms of a stress-induced affective disorder.

The present disclosure provides for a method for preventing or delaying a stress-induced affective disorder or stress-induced psychopathology in a subject in need thereof. The method may comprise administering an effective amount of a pharmaceutic composition comprising an NMDA receptor antagonist such as fluoroethylnormemantine, or a pharmaceutically acceptable salt or solvate thereof, analog, derivative, or metabolite thereof, to a subject prior to, during, an/or after a stressor.

The present disclosure also provides for a method for inducing and/or enhancing stress resilience in a subject in need thereof. The method may comprise administering an effective amount of a pharmaceutic composition comprising an NMDA receptor antagonist such as fluoroethylnormemantine, or a pharmaceutically acceptable salt or solvate thereof, or analog, derivative, or metabolite thereof, to a subject prior to, during, an/or after a stressor.

The present agent/composition may be administered by any method known in the art, including, without limitation, intranasal, oral, transdermal, ocular, intraperitoneal, inhalation, intravenous, intracerebroventricular (ICV), intracisternal injection or infusion, subcutaneous, implant, vaginal, sublingual, urethral (e.g., urethral suppository), subcutaneous, intramuscular, intravenous, rectal, sub-lingual, mucosal, ophthalmic, spinal, intrathecal, intra-articular, intra-arterial, sub-arachinoid, bronchial and lymphatic administration. Topical formulation may be in the form of gel, ointment, cream, aerosol, etc.; intranasal formulation can be delivered as a spray or in a drop; transdermal formulation may be administered via a transdermal patch or iontorphoresis; inhalation formulation can be delivered using a nebulizer or similar device. Compositions can also take the form of tablets, pills, capsules, semisolids, powders, sustained release formulations, solutions, suspensions, elixirs, aerosols, or any other appropriate compositions.

In certain embodiments, an effective amount of the present agent is a sub-anesthetic amount of an NMDA receptor antagonist such as fluoroethylnormemantine, or a pharmaceutically acceptable salt or solvate thereof, or a derivative or analog thereof, or a metabolite thereof. In certain embodiments, an effective amount of the present agent is a sub-analgesic amount of an NMDA receptor antagonist such as fluoroethylnormemantine, or a pharmaceutically acceptable salt or solvate thereof, or a derivative or analog thereof, or a metabolite thereof.

In certain embodiments, a subject is treated with the present agent/composition via intravenous, oral, transdermal or intranasal administration. In certain embodiments, a subject is injected with the present agent/composition.

In certain embodiments, a subject is treated with a single dose of an effective amount of the present agent/composition, prior to, during, and/or after a stressor. In some aspects, a subject is treated with multiple doses of an effective amount of the present agent/composition, prior to, during, and/or after a stressor.

In certain embodiments, an NMDA receptor antagonist, such as FENM, or a pharmaceutically acceptable salt or solvate thereof, an analog thereof, a derivative thereof, or a metabolite thereof, is administered in a composition comprising a pharmaceutically acceptable carrier, excipient or diluent. Also provided herein is a pharmaceutical composition that comprises an NMDA receptor antagonist, such FENM, or a pharmaceutically acceptable salt or solvate thereof, an analog thereof, a derivative thereof, or a metabolite thereof, and a pharmaceutically acceptable carrier, excipient or diluent, for use in the prophylactic treatment of a stress-induced affective disorder.

“Patient” or “subject” refers to mammals and includes human and veterinary subjects. In certain embodiments, the subject is a mammal. A subject may be a human or vertebrate animal or mammal, including but not limited to a mouse, rat, dog, cat, horse, cow, pig, sheep, goat, turkey, chicken, and primate, e.g., monkey.

The present agent (e.g., an NMDA receptor antagonist) may antagonize the NMDA receptor through any mechanism, including, but not limited to, inhibiting/decreasing NMDA receptor activity, decreasing NMDA receptor level, and/or inhibiting/decreasing NMDA receptor gene expression. The terms “antagonist of NMDAR”, “antagonist of the NMDA receptor”, “NMDA receptor antagonist”, and “NMDAR antagonist” are used interchangeably herein.

By “inhibition”, “down-regulation” or “decrease” is meant any negative effect on the condition being studied; this may be total or partial. Thus, where the level or activity of a protein (e.g., NMDA receptor or NMDAR) is being detected, the present agent/composition is capable of inhibiting, down-regulating, or decreasing the level or activity of the protein (e.g., NMDA receptor or NMDAR). The inhibition or down-regulation of the level or activity of the protein achieved by the present agent may be at least 10%, such as at least 20%, at least 30%, at least 40%, at least 50%, at least 60%, at least 70%, at least 80%, at least 90% or more compared to the level or activity of the protein (e.g., NMDA receptor or NMDAR) in the absence of the present agent/composition.

The term “derivative” of the present agent may refer to pharmaceutically acceptable derivatives, which can be any pharmaceutically acceptable salt, solvate, prodrug, e.g. ester, or other precursors, of a compound which upon administration to the recipient is capable of providing (directly or indirectly) the active compound or an active metabolite or residue thereof. Such salts include pharmaceutically acceptable basic or acid addition salts as well as pharmaceutically acceptable metal salts, ammonium salts and alkylated ammonium salts. Such derivatives are recognizable to those skilled in the art, without undue experimentation. Derivatives are described, for example, in Burger's Medicinal Chemistry and Drug Discovery, 5th Edition, Vol 1: Principles and Practice, which is incorporated herein by reference. In certain embodiments, pharmaceutically acceptable derivatives include salts, solvates, esters, carbamates, and phosphate esters.

The present agent/composition may be administered by various routes, including oral, intravenous (i.v. or IV), intranasal (i.n. or IN), intramuscular (i.m. or IM), caudal, intrathecal, and subcutaneous (s.c.) routes.

Fluoroethylnormemantine Fluoroethylnormemantine (FENM or FNM) is an antagonist of the N-methyl-D-aspartate (NMDA) receptor (NMDAR).

In one embodiment, fluoroethylnormemantine (FENM or FNM) is 1-amino-3-fluoroethyl-adamantane.

In one embodiment, fluoroethylnormemantine (FENM or FNM) has the structure of Compound (I):

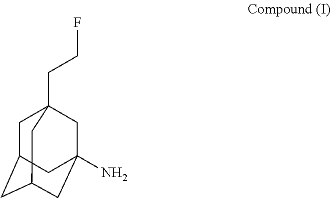 [SMILES: NC12CC3CC(C1)CC(CCF)(C3)C2]

In certain embodiments, fluoroethylnormemantine's salt may have the structure of Compound (II), wherein X indicates a counter anion from a biological environment or selected from ions including, but not limited to, chloride, bromide, iodide, acetate, methane sulphonate, benzene sulphonate, camphosulphonate, tartrate, dibenzoate, ascorbate, fumarate, citrate, phosphate, salicylate, oxalate, bromohydrate, or tosylate:

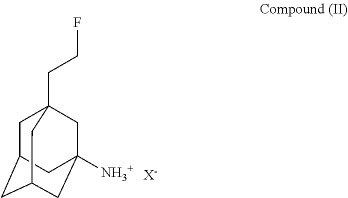

Non-limiting examples of the pharmaceutically acceptable salts of fluoroethylnormemantine include, fluoroethylnormemantine chloride, fluoroethylnormemantine bromide, fluoroethylnormemantine iodide, fluoroethylnormemantine acetate, fluoroethylnormemantine methane sulphonate, fluoroethylnormemantine benzene sulphonate, c fluoroethylnormemantine amphosulphonate, fluoroethylnormemantine tartrate, fluoroethylnormemantine dibenzoate, fluoroethylnormemantine ascorbate, fluoroethylnormemantine fumarate, fluoroethylnormemantine citrate, fluoroethylnormemantine phosphate, fluoroethylnormemantine salicylate, fluoroethylnormemantine oxalate, fluoroethylnormemantine bromohydrate, and fluoroethylnormemantine tosylate. In one embodiment, the pharmaceutically acceptable salt of fluoroethylnormemantine is fluoroethylnormemantine chloride, i.e., FENM-HCl. In one embodiment, the pharmaceutically acceptable salt of fluoroethylnormemantine is 1-amino-3-fluoroethyl-adamantane hydrochloride.

Methods of synthesizing fluoroethylnormemantine or its salts can be found in U.S. Pat. No. 9,714,212.

### NMDA Receptor Antagonists

NMDA receptor antagonists are compounds that antagonize, or inhibit, the action of the NMDA receptor. An NMDA receptor antagonist may be a competitive antagonist, an uncompetitive antagonist, a noncompetitive antagonist, and/or a glycine antagonist.

Non-limiting examples of NMDA receptor antagonists include, Fluoroethylnormemantine (FENM or FNM), ketamine, dextromethorphan (DXM), histogranin, memantine, meperidine, methadone, methoxetamine (MXE), phencyclidine (PCP), nitrous oxide (N₂O), AP5 (APV, R-2-amino-5-phosphonopentanoate), AP7 (2-amino-7-phosphonoheptanoic acid), CPPene ((3-[(R)-2-carboxypiperazin-4-yl]-prop-2-enyl-1-phosphonic acid), Selfotel, Amantadine, Atomoxetine, AZD6765, Agmatine, chloroform, dextrallorphan, dextromethorphan, dextrorphan, diphenidine, dizocilpine (MK-801), ethanol, eticyclidine, gacyclidine, ibogaine, magnesium, memantine, nitromemantine, rolicyclidine, tenocyclidine, methoxydine, tiletamine, neramexane, eliprodil, dexoxadrol, etoxadrol, remacemide, delucemine, WMS-2539, NEFA, 8A-PDHQ, HU-211, Aptiganel (Cerestat, CNS-1102), rhynchophylline, kynurenic acid, Rapastinel (GLYX-13), NRX-1074, 7-Chlorokynurenic acid, 4-Chlorokynurenine (AV-101), TK-40, 1-Aminocyclopropanecarboxylic acid (ACPC), L-Phenylalanine, Xenon, or analogs or derivatives thereof. Ketamine derivatives such as Rapastinel or Glyx-13 are also included. Rapastinel is an NMDA receptor glycine site partial agonist. It is an amidated tetrapeptide (Thr-Pro-Pro-Thr-NH₂) which rapidly crosses the blood brain barrier, but is not active orally.

Non-limiting examples of the NMDA receptor antagonists also include anti-receptor antibodies, anti-ligand antibodies, inhibitory nucleic acids, etc.

Several synthetic opioids function as NMDA receptor-antagonists, such as pethidine, methadone, meperidine, dextropropoxyphene, tramadol, levorphanol, and ketobemidone.

### Pharmaceutical Compounds

The agents used in the present methods include all hydrates, solvates, and complexes of the compounds described herein. If a chiral center or another form of an isomeric center is present in the present compound, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present disclosure may be in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this disclosure. In cases wherein compounds may exist in tautomeric forms, such as keto-enol tautomers, each tautomeric form is contemplated as being included within this disclosure whether existing in equilibrium or predominantly in one form.

When the structure of the compounds used in this invention includes an asymmetric carbon atom such compound can occur as racemates, racemic mixtures, and isolated single enantiomers. All such isomeric forms of these compounds are expressly included in this invention. Each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

The present disclosure is also intended to include use of all isotopes of atoms occurring on the compounds disclosed herein. Isotopes include those atoms having the same atomic number but different mass numbers. Isotopically-labeled compounds can generally be prepared by conventional techniques known to those skilled in the art or by processes analogous to those described herein using an appropriate isotopically-labeled reagents in place of the non-labeled reagents employed.

The compounds of the instant disclosure may be in a salt form. As used herein, a “salt” is a salt of the instant compound which has been modified by making acid or base, salts of the compounds. In the case of compounds used for treatment of mammals, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately treating a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The present methods also encompass administering a physiologically functional derivative of the present compound. As used herein, the term “physiologically functional derivative” refers to a compound (e.g., a drug precursor) that is transformed in vivo to yield the present compound or its active metabolite, or a pharmaceutically acceptable salt, hydrate or solvate of the compound. The transformation may occur by various mechanisms (e.g., by metabolic or chemical processes), such as, for example, through hydrolysis in blood. Prodrugs are such derivatives, and a discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

### Dosages

In certain embodiments, the effective amount of the present agent is a dose of about 0.01 to about 3 mg per kilogram of body weight of the subject (mg/kg), i.e., from about 0.01 mg/kg to about 3 mg/kg body weight. In certain embodiments, the effective amount of the present compound ranges 0.001 to approximately 3 mg/kg body weight, 0.001 to approximately 2 mg/kg body weight, from about 0.01 mg/kg to about 3 mg/kg body weight, from about 0.01 to about 2 mg/kg of body weight, about 0.01 to about 1.5 mg/kg of body weight, about 0.05 to about 1.4 mg/kg of body weight, about 0.05 to about 1.3 mg/kg of body weight, about 0.05 to about 1.2 mg/kg of body weight, about 0.05 to about 1.1 mg/kg of body weight, about 0.01 to about 1 mg/kg of body weight, or about 0.05 to about 0.7 mg/kg of body weight. In some aspects, the dose is about 0.05 to about 0.5 mg/kg. In some aspects, the dose is less than about 0.5 mg/kg, less that about 0.4 mg/kg, or less than about 0.3 mg/kg body weight. In some aspects, the effective amount of the present compound is a dose in the range of from about 0.01 mg/kg to about 1.5 mg/kg body weight. In some aspects, the effective amount of the present compound is a dose in the range of from about 0.01 mg/kg to about 1 mg/kg body weight. In some aspects, the effective amount of the present compound is a dose in the range of from about 0.01 mg/kg to about 0.75 mg/kg body weight. In some aspects, the effective amount of the present compound is a dose in the range of from about 0.75 mg/kg to about 1.5 mg/kg body weight. In some aspects, the effective amount of the present compound is a dose in the range of from about 0.5 mg/kg to about 1.2 mg/kg body weight. In some aspects, the effective amount of the present compound is a dose in the range of from about 0.05 mg/kg to about 0.5 mg/kg. In some aspects, the effective amount of the present compound is a dose of about 0.2 mg/kg or about 0.4 mg/kg body weight. In some aspects, the dose of the present compound is, about 0.01 to about 1 mg/kg, about 0.1 to about 0.5 mg/kg, about 0.8 to about 1.2 mg/kg, about 0.7 to about 1.1 mg/kg, about 0.05 to about 0.7 mg/kg, about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, or about 3 mg/kg body weight.

In certain embodiments, the dose of the present compound per administration is from about 1 to about 250 mg, from about 10 mg to about 300 mg, about 10 mg to about 250 mg, about 10 to about 200 mg, about 15 to about 175 mg, about 20 to about 175 mg, about 8 mg to about 32 mg, about 50 mg to about 75 mg, about 25 to about 150 mg, about 25 to about 125 mg, about 25 to about 100 mg, about 50 to about 100 mg, about 50 mg to about 75 mg, about 75 mg to about 100 mg, or about 75 mg to about 200 mg, about 1 mg, 2 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, and 250 mg. In some aspects, the dose of the present compound is about 50 mg. In some aspects, the dose of the present compound is about 75 mg. In some aspects, the total dose of the present compound is about 100 mg.

In some aspects, the therapeutically effective amount of the present compound is a sub-anesthetic dose. In some aspects, the therapeutically effective amount of the present compound is a sub-analgesic dose. In certain embodiments, the therapeutically effective amount of the present agent is below the level that results in one or more side effects of the agent. In certain embodiments, the therapeutically effective amount of the present compound is an anesthetic dose or analgesic dose.

In some aspects, the (therapeutically) effective amount of the present agent is about 0.01 mg to about 1000 mg, from about 0.01 mg to about 500 mg, from about 0.1 mg to about 250 mg, or any amount or range therein. In another aspect, the (therapeutically) effective amount of the present agent is, e.g., 0.01 mg, 0.025 mg, 0.05 mg, 0.1 mg, 0.5 mg, 1 mg, 5 mg, 10 mg, 25 mg, 50 mg, 55 mg, 60 mg, 65 mg, 70 mg, 75 mg, 80 mg, 90 mg, 95 mg, 100 mg, 150 mg, 200 mg, 250 mg, or 500 mg.

In certain embodiments, a therapeutically effective dose of the present agent may be adjusted depending on conditions of the disease/disorder to be treated or prophetically treated, the age, body weight, general health conditions, sex, and diet of the subject, dose intervals, administration routes, excretion rate, and combinations of drugs.

An initial dose of the present agent may be larger, followed by one or more smaller maintenance doses. Other ranges are possible, depending on the subject's response to the treatment. An initial dose may be the same as, or lower or higher than subsequently administered doses.

The present agent/composition may be administered daily, weekly, biweekly, several times daily, semi-weekly, every other day, bi-weekly, quarterly, several times per week, semi-weekly, monthly etc. The duration and frequency of treatment may depend upon the subject's response to treatment.

In certain embodiments, a subject may be administered 1 dose, 2 doses, 3 doses, 4 doses, 5 doses, 6 doses or more of the present agent/composition. In certain embodiments, a single dose of the present agent/composition is administered in the present method. In certain embodiments, multiple doses of the present agent/composition (e.g., 2 doses, 3 doses, 4 doses, 5 doses, 6 doses, 7 doses, 8 doses, 9 doses, 10 doses or more) are administered in the present method.

In certain embodiments, when there are more than one doses of the present agent/composition administered to a subject, the second dose is lower than the first dose. In certain embodiments, the second dose is an amount that is at most one-half, one-quarter, or one-tenth the amount of the first dose.

The number and frequency of doses may be determined based on the subject's response to administration of the composition, e.g., if one or more of the patient's symptoms improve and/or if the subject tolerates administration of the composition without adverse reaction.

In certain embodiments, the present agent/composition is administered at least once a day, at least twice a day, at least three times per day, or more. In certain embodiments, the present agent/composition is administered at least once a week, at least twice a week, at least three times per week, or more frequently. In certain embodiments, the present agent/composition is administered at least twice per month, or at least once per month. Treatment using the present method can continue as long as needed.

### Dosing Time Frame

In certain embodiments, the present agent/composition is administered to a subject prior to a stressor. In certain embodiments, the present agent/composition is administered to a subject during a stressor. In certain embodiments, the present agent/composition is administered to a subject prior to, during, and/or after a stressor. In certain embodiments, the present agent/composition is administered to a subject both prior to and after a stressor. In certain embodiments, the present agent/composition is administered to a subject after a stressor. In certain embodiments, the present agent/composition is administered to a subject prior to a stressor, and again prior to a recurrence of the stressor or a different stressor.

In certain embodiments, the present agent/composition is administered to the subject about 12 hours to about 4 weeks, about 18 hours to about 4 weeks, about 1 day to about 3.5 weeks, about 2 days to about 3 weeks, about 3 days to about 3 weeks, about 4 days to about 3 weeks, about 5 days to about 3 weeks, about 6 days to about 3 weeks, about 2 days to about 2.5 weeks, about 3 days to about 2.5 weeks, about 4 days to about 2.5 weeks, about 5 days to about 2.5 weeks, about 6 days to about 2.5 weeks, about 1 week to about 2.5 weeks, about 1 week to about 2.5 weeks, about 1 week to about 2 weeks, about 5 minutes to about 3 days, about 10 minutes to about 2 days, about 15 minutes to about 24 hours, about 20 minutes to about 12 hours, about 30 minutes to about 8 hours, about 45 minutes to about 5 hours, about 1 hour to about 12 hours, about 2 hours to about 5 hours, at least, within or about 5 minutes, at least, within or about 10 minutes, at least, within or about 15 minutes, at least, within or about 20 minutes, at least, within or about 30 minutes, at least, within or about 45 minutes, at least, within or about 1 hour, at least, within or about 2 hours, at least, within or about 3 hours, at least, within or about 4 hours, at least, within or about 5 hours, at least, within or about 6 hours, at least, within or about 8 hours, at least, within or about 10 hours, at least, within or about 12 hours, at least, within or about 15 hours, at least, within or about 1 day, at least, within or about 1.5 days, at least, within or about 2 days, at least, within or about 3 days, at least, within or about 4 days, at least, within or about 5 days, at least, within or about 6 days, at least, within or about 1 week, at least, within or about 8 days, at least, within or about 9 days, at least, within or about 10 days, at least, within or about 11 days, at least, within or about 12 days, at least, within or about 13 days, at least, within or about 2 weeks, at least, within or about 2.5 weeks, at least, within or about 3 weeks, at least, within or about 3.5 weeks, or at least, within or about 4 weeks, prior to, and/or after, a stressor.

In certain embodiments, the present agent/composition can be used to treat numerous stress-induced psychiatric diseases as a prophylactic and/or antidepressant. For example, the present agent/composition (e.g., FENM) attenuates learned fear and protects against stress-induced depressive-like behavior. FENM may be administered at least about one week before exposure to a stressor. In various embodiments, FENM can be administered at least about 1, 2, 3, 4, 5, 6, 7, 8, 9, 10, 11, 12, 13, 14, 15, 16, 17, 18, 19, 20, 21, 22, 23, 24, or 25 weeks before an exposure to a stressor.

The present agent/composition also attenuates learned fear and protects against stress-induced depressive-like behavior when administered after an exposure to a stressor. For example, the present agent/composition (e.g., FENM) can be administered about 1, 2, 3, 4, 5, 6, 7, 8, 9, 10, 15, 20, 30, 40, 50, 60, 70, 80, 90, 100, 150, 200, 250, 300, 350, 400, 450, 500 minutes after an exposure to a stressor.

In certain embodiments, the administration of the present agent/composition is continued over a period of up to 2 days, up to 3 days, up to 4 days, up to 5 days, up to 6 days, up to 1 week, up to 2 weeks, up to 3 weeks, up to 4 weeks, about 2 days, about 3 days, about 4 days, about 5 days, about 6 days, about 1 week, about 2 weeks, about 3 weeks, about 4 weeks, about 5 weeks, about 6 weeks, about 7 weeks, about 8 weeks, about 9 weeks, about 10 weeks, or longer.

In certain embodiments, the present agent/composition is administered once, twice, at least twice, at least three times, at least four times, at least five time, at least six times, at least seven times, at least eight times, at least nine times, or more per treatment.

In certain embodiments, the present agent/composition is administered at least once a day, at least twice a day, at least three times per day, at least once a week, at least twice a week, at least three times a week, at least once per month, at least twice per month, or more frequently. Treatment can continue as long as needed. The present agent/composition may be administered daily, weekly, biweekly, several times daily, semi-weekly, every other day, bi-weekly, quarterly, several times per week, semi-weekly, monthly etc. The duration and frequency of treatment may depend upon the subject's response to treatment.

### Stressors

A stressor is a stimulus that causes stress. It can be an event or other factor that disrupts the body's homeostasis of temperature, blood pressure, and/or other functions. In certain embodiments, a stressor is a traumatic or stressful event. Because humans have sophisticated brains and thought processes, anticipating a disruption can also be a stressor. In certain embodiments, a stressor is injury, trauma, combat, warfare, surgery, an accident, a criminal assault, child abuse, natural or human-caused disasters, a crash, grief, hunger, heat, cold, chemical exposure, autoimmune disease, infectious disease, viral infection, cancer, exhaustion, physical distress, neuropathy, hyperalgesia, allodynia, emotional distress, or depression. A traumatic event may be an event or something that threatens the person's life or the life of a close one or it could be something witnessed. U.S. Patent Application No. 20140018339.

A stressor may refer to a physical, chemical or emotional factor or combination of factors that causes bodily or mental tension and that may be a factor in disease causation. The term “stressor” is used interchangeably with the term, “stress”, “stress event”, “stressful event”, “stress-induced event”, or “stress-related event”. It should be appreciated that any form of stress can be compatible with aspects of the invention. A stressor may be acute, or may be chronic. Exposure to stress can be chronic or acute. As used here, “chronic stress” may refer to a state of prolonged tension from internal or external stressors, which may cause various physical and/or psychiatric manifestations. The effects of chronic and acute stress can be different. Several non-limiting examples of situations where a subject could be exposed to chronic stress include, military service such as a combat mission, and natural disasters, such as participation in a search-and-rescue operation or rebuilding following a natural disaster. These examples are encompassed within the definition of stress-associated disorders, as used herein.

There are numerous physiological processes that are altered in response to stress. Among these are altered cortisol, corticotropin, catecholamine and serotonin levels. These levels return to baseline after an acute stressor is removed (McEwen N Eng J Med 1998 338(3):171-179). These biochemical markers of stress in turn lead to ill health and psychosocial disorders. Consequently, stress plays a major role in physical and mental health. Stress can affect the onset of, or susceptibility to disease. It can also affect the progression or course of disease even when there is another underlying pathophysiology of the disease. Recovery from an existing disease can also be delayed due to stress. For example, stress is a contributing factor to high blood pressure, heart disease, headaches, colitis, irritable bowel syndrome, temporo-mandibular joint disorder, cancer, peptic ulcers, insomnia, skin disorders and asthma. Stress can also aggravate other conditions such as multiple sclerosis, diabetes, herpes, mental illness, substance abuse and psychiatric disorders characterized by the presence of violent or aggressive tendencies. Particularly, stress contributes to functional somatic disorders, affective disorders and major depressive disorder (MDD). These include disorders such as chronic fatigue syndrome (CFS), fibromyalgia (FMS), Gulf War Syndrome, anxiety, and post-traumatic stress disorder (PTSD). Stressors that disrupt normal exercise or sleep patterns.

Additional examples of use include administration prior to military deployment to protect service members (active combat soldiers, battlefield surgeons, etc.) and even military working dogs against stress. Potential non-military use cases include, but are not limited to: police, firefighters, first responders, emergency medical technicians (EMTs), emergency room (ER) doctors, prison guards (and prisoners), humanitarian aid workers, and refugees.

In certain embodiments, a subject may be administered the present agent or composition prior to a situation in which the subject (such as an early responder or military personnel) is likely to be exposed to traumatic stress, immediately after exposure to traumatic stress, and/or when the subject feels that his or her PTSD symptoms are likely to appear.

### Resilience to Stress

Resilience to stress refers to the capacity of a subject to adapt or change successfully, and/or to maintain physiological, neurological, or psychological homeostasis, in the face of a stressor (e.g., adversity). As used herein, the term “enhancing resilience” refers to increasing the ability of a subject to experience a stressor (e.g., a traumatic event) without suffering a stress-induced affective disorder, and/or with less post-event symptomatology or disruption of homeostasis and/or normal activities of daily living. In certain embodiments, improving resilience can prevent a stress-induced affective disorder. In certain embodiments, improving resilience can reduce at least one of the signs, symptoms, or symptom clusters of a stress-induced affective disorder. In certain embodiments, the present method enhances a subject's resilience to stress, helps protect against developing stressor-related psychopathology, decrease the functional consequences of stressor-induced disorders (e.g., PTSD, etc.), and reduce medical morbidity and mortality.

The Connor-Davidson Resilience Scale (CD-RISC) is a 25-item self-report scale, each rated on a 5-point scale (0-4), with higher scores reflecting greater resilience (Connor K M & Davidson, J R T. Development of a new resilience scale: the Connor-Davidson Resilience Scale (CD-RISC). Depression and Anxiety, 2003: 18: 71-82).

Resilience, psychological growth and life satisfaction may be measured with the CD-RISC, the Purpose in Life Scale, the abbreviated MOS Social Support Survey, the PTGI, and the Q-LES-Q.

### Combination Therapy

The present agent or composition may be administered to a subject alone, or may be administered to a subject in combination with one or more other treatments/agents.

In certain embodiments, the second agent is an anti-depressant, an anxiolytic, or combinations thereof. In certain embodiments, the second agent is a serotonin reuptake inhibitor (SRI), or a selective serotonin reuptake inhibitor (SSRI). In certain embodiments, the second agent is fluoxetine, paroxetine, sertraline, lithium, riluzole, prazosin, lamotrigine, ifenprodil, or combinations thereof. In certain embodiments, the second agent is a dual serotonin norepinephrine reuptake inhibitor compound (DRI). In certain embodiments, the second agent is venlafaxine, duloxetine, milnacipran, or combinations thereof. In certain embodiments, the second agent is a non-tricyclic triple reuptake inhibitor (TRI).

In certain embodiments, the present agent or composition is administered to a subject in combination with one or more treatments/agents such as antidepressants, analgesics, muscle relaxants, anorectics, stimulants, antiepileptic drugs, and sedative/hypnotics. Non-limiting examples of compounds that can be administered in combination with the present compound or composition include, neurontin, pregabalin, pramipexole, L-DOPA, amphetamine, tizanidine, clonidine, tramadol, morphine, tricyclic antidepressants, codeine, carbamazepine, sibutramine, amphetamine, valium, trazodone and combinations thereof.

In certain embodiments, combination therapy means simultaneous administration of the agents in the same dosage form, simultaneous administration in separate dosage forms, or separate administration of the agents.

In certain embodiments, the second agent/treatment is used as adjunctive therapy to the present agent or composition. In certain embodiments, the treatment includes a phase wherein treatment with the second agent/treatment takes place after treatment with the present agent or composition has ceased. In certain embodiments, the treatment includes a phase where treatment with the present agent or composition and treatment with the second agent/treatment overlap.

Combination therapy can be sequential or can be administered simultaneously. In either case, these drugs and/or therapies are said to be “co-administered.” It is to be understood that “co-administered” does not necessarily mean that the drugs and/or therapies are administered in a combined form (i.e., they may be administered separately (e.g., as separate compositions or formulations) or together (e.g., in the same formulation or composition) to the same or different sites at the same or different times).

In certain embodiments, a subject is treated concurrently (or concomitantly) with the present agent or composition and a second agent. In certain embodiments, a subject is treated initially with the present agent or composition, followed by cessation of the present compound or composition treatment and initiation of treatment with a second agent. In certain embodiments, the present agent or composition is used as an initial treatment, e.g., by administration of one, two or three doses, and a second agent is administered to prolong the effect of the present compound or composition, or alternatively, to boost the effect of the present agent or composition. A person of ordinary skill in the art will recognize that other variations of the presented schemes are possible, e.g., initiating treatment of a subject with the present agent or composition, followed by a period wherein the subject is treated with a second agent as adjunct therapy to the present agent or composition treatment, followed by cessation of the present agent or composition treatment.

The present agent and the other pharmaceutically active agent(s) may be administered together or separately and, when administered separately this may occur simultaneously or sequentially in any order. The amounts of the present agent and the other pharmaceutically active agent(s) and the relative timings of administration will be selected in order to achieve the desired combined therapeutic effect.

In various embodiments, the therapies (e.g., a composition provided herein and a second agent in a combination therapy) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In certain embodiments, the therapies are administered no more than 24 hours apart or no more than 48 hours apart. In certain embodiments, two or more therapies are administered within the same patient visit. In other embodiments, the composition provided herein and the second agent are administered concurrently. In other embodiments, the composition provided herein and the second agent are administered at about 2 to 4 days apart, at about 4 to 6 days apart, at about 1 week part, at about 1 to 2 weeks apart, or more than 2 weeks apart. In certain embodiments, administration of the same agent may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same agent may be repeated and the administration may be separated by at least at least 1 day, 2 days, 3 days, 5 days, 10 days, 15 days, 30 days, 45 days, 2 months, 75 days, 3 months, or 6 months. In certain embodiments, a composition provided herein and a second agent are administered to a subject in a sequence and within a time interval such that the composition provided herein can act together with the other agent to provide an increased benefit than if they were administered otherwise. For example, the second active agent can be administered at the same time or sequentially in any order at different points in time; however, if not administered at the same time, they should be administered sufficiently close in time so as to provide the desired therapeutic or prophylactic effect. In one embodiment, the composition provided herein and the second active agent exerts their effect at times which overlap. Each second active agent can be administered separately, in any appropriate form and by any suitable route. In other embodiments, the composition provided herein is administered before, concurrently or after administration of the second active agent. The term “about” refers to +10% of the referenced value. In other embodiments, courses of treatment are administered concurrently to a patient, i.e., individual doses of the second agent are administered separately yet within a time interval such that the compound provided herein can work together with the second active agent. For example, one component can be administered once per week in combination with the other components that can be administered once every two weeks or once every three weeks. In other words, the dosing regimens are carried out concurrently even if the therapeutics are not administered simultaneously or during the same day. The second agent can act additively or synergistically with the compound provided herein. In one embodiment, the composition provided herein is administered concurrently with one or more second agents in the same pharmaceutical composition. In another embodiment, a composition provided herein is administered concurrently with one or more second agents in separate pharmaceutical compositions. In still another embodiment, a composition provided herein is administered prior to or subsequent to administration of a second agent. Also contemplated are administration of a composition provided herein and a second agent by the same or different routes of administration, e.g., oral and parenteral. In certain embodiments, when the composition provided herein is administered concurrently with a second agent that potentially produces adverse side effects including, but not limited to, toxicity, the second active agent can advantageously be administered at a dose that falls below the threshold that the adverse side effect is elicited.

Encompassed by the present disclosure are agents/compositions and methods to prophylactically treat a subject prior to a stressor. In certain embodiments, the present agent/composition and method prevent or delay a stress-induced affective disorder or stress-induced psychopathology in a subject.

The present disclosure also provides for agents/compositions and methods to treat a stress-induced affective disorder or stress-induced psychopathology in a subject.

In certain embodiments, stress-induced affective disorders include major depressive disorder and posttraumatic stress disorder.

### Stress-Induced Affective Disorders

There are numerous disorders that are either caused by or exacerbated by stress. The present agent/composition and method may prevent, delay and/or treat a stress-induced affective disorder or stress-induced psychopathology. Stress-induced affective disorders or stress-induced psychopathologies any condition, disease or disorder that results, at least in part, from exposure to stress or is exacerbated, at least in part, from exposure to stress.

Stress-induced affective disorders or stress-induced psychopathologies which may be prevented or treated by the present agent/composition and method include, but are not limited to, depression, depressive-like behavior, fear, major depressive disorder, addictive disorders such as substance abuse, anorexia, bulimia, obesity, smoking addiction, and weight addiction; anxiety disorders such as agoraphobia, generalized anxiety disorder, obsessive compulsive disorder, panic attacks, performance anxiety, phobias, and post-traumatic stress disorder (PTSD); psychiatric disorders such as stress-induced psychiatric disorders; bipolar disorder, acute stress disorder, obsessive-compulsive disorder, social anxiety disorders, panic disorders, schizophrenia, phobias, obsessive compulsive disorders, Trichotillomania, autoimmune diseases such as allergies, arthritis, fibromyalgia, fibromytosis, lupus, multiple sclerosis, rheumatoid arthritis, Sjogren's syndrome, and vitiligo; cancer such as bone cancer, brain cancer, breast cancer, cervical cancer, colon cancer, Hodgkin's disease, leukemia, liver cancer, lung cancer, lymphoma, multiple myeloma, ovarian cancer, pancreatic cancer, and prostate cancer; cardiovascular disorders such as arrhythmia, arteriosclerosis, Burger's disease, essential hypertension, fibrillation, mitral valve prolapse, palpitations, peripheral vascular disease, Raynaud's disease, stroke, tachycardia, and Wolff-Parkinson-White Syndrome; and developmental disorders such as attention deficit disorder, concentration problems, conduct disorder, dyslexia, hyperkinesis, language and speech disorders, and learning disabilities.

### Anxiety Disorders

The present agent/composition and method may prevent, delay and/or treat an anxiety disorder. The five major types of anxiety disorders are: panic disorder, obsessive-compulsive disorder, post-traumatic stress disorder, generalized anxiety disorder and phobias (including social phobia, also called social anxiety disorder). Each anxiety disorder has its own distinct features, but they are all bound together by the common theme of excessive, irrational fear and dread. It is common for an anxiety disorder to accompany depression, eating disorders, substance abuse, or another anxiety disorder.

Panic disorder is characterized by repeated episodes of intense fear that strike often and without warning. Physical symptoms include chest pain, heart palpitations, shortness of breath, dizziness, abdominal distress, feelings of unreality, and fear of dying. Obsessive-compulsive disorder is characterized by repeated, unwanted thoughts or compulsive behaviors that seem impossible to stop or control. Generalized Anxiety Disorder is characterized by exaggerated worrisome thoughts and tension about everyday routine life events and activities, lasting at least six months. Almost always anticipating the worst even though there is little reason to expect it; accompanied by physical symptoms, such as fatigue, trembling, muscle tension, headache, or nausea. Phobias are characterized into two major types of phobias, social phobia and specific phobia. People with social phobia have an overwhelming and disabling fear of scrutiny, embarrassment, or humiliation in social situations, which leads to avoidance of many potentially pleasurable and meaningful activities. People with specific phobia experience extreme, disabling, and irrational fear of something that poses little or no actual danger; the fear leads to avoidance of objects or situations and can cause people to limit their lives unnecessarily.

### Posttraumatic Stress Disorder (PTSD)

Typically, a subject suffering from PTSD was exposed to a traumatic event in which the person experienced, witnessed, or was confronted with an event or events that involved actual or threatened death or serious injury, or a threat to the physical integrity of self or others and the person's response involved intense fear, helplessness, or horror.

Having repeated intrusive memories of the trauma exposure is one of the core symptoms of PTSD. Patients with PTSD are known to display impairments in learning and memory during neuropsychological testing. Other core symptoms of PTSD include heightened stress sensitivity (startle), tension and anxiety, memory disturbances, and dissociation.

In certain embodiments, the present method prevents or inhibits the development of post-traumatic stress disorder (PTSD) in a subject. In certain embodiments, the present method prevents or inhibits the development of one or more PTSD-like symptoms. In certain embodiments, a subject may be administered the present agent or composition prior to a situation in which the subject (such as an early responder or military personnel) is likely to be exposed to traumatic stress, immediately after exposure to traumatic stress, and/or when the subject feels that his or her PTSD symptoms are likely to appear.

Typically, the traumatic event is persistently re-experienced in one or more of the following ways: recurrent and intrusive distressing recollections of the event, including images, thoughts, or perceptions, recurrent distressing dreams of the event, acting or feeling as if the traumatic event were recurring (includes a sense of reliving the experience, illusions, hallucinations, and dissociative flashback episodes, including those that occur on awakening or when intoxicated), intense psychological distress at exposure to internal or external cues that symbolize or resemble an aspect of the traumatic event, physiological reactivity on exposure to internal or external cues that symbolize or resemble an aspect of the traumatic event. An individual suffering from PTSD also has persistent avoidance of stimuli associated with the trauma and numbing of general responsiveness (not present before the trauma), as indicated by 3 or more of the following: efforts to avoid thoughts, feelings, or conversations associated with the trauma, efforts to avoid activities, places, or people that arouse recollections of the trauma, inability to recall an important aspect of the trauma, significantly diminished interest or participation in significant activities, feeling of detachment or estrangement from others, restricted range of affect (e.g., unable to have loving feelings), sense of a foreshortened future (e.g., does not expect to have a career, marriage, children, or a normal life span), persistent symptoms of increased arousal (not present before the trauma), as indicated by 2 or more of the following: difficulty falling or staying asleep, irritability or outbursts of anger, difficulty concentrating, hypervigilance, exaggerated startle response. The disturbance, which has lasted for at least a month, causes clinically significant distress or impairment in social, occupational, or other important areas of functioning.

In certain embodiments, the present compound or composition prevents, reduces, eliminates or delays one or more of the symptoms including, but not limited to, re-experiencing of the traumatic experience in the form of intrusive memories, nightmares, flashbacks; emotional and physical reactions triggered by reminders of the trauma; distancing from others; decreased interest in activities and other people; numbing of feelings; avoidance of trauma reminders; hyperarousal symptoms, including disrupted sleep, irritability, hypervigilance, decreased concentration; increased startle reflex; and combinations thereof.

Whatever the source of the problem, some people with PTSD repeatedly relive the trauma in the form of nightmares and disturbing recollections during the day. They may also experience other sleep problems, feel detached or numb, or be easily startled. They may lose interest in things they used to enjoy and have trouble feeling affectionate. They may feel irritable, more aggressive than before, or even violent. Things that remind them of the trauma may be very distressing, which could lead them to avoid certain places or situations that bring back those memories.

The disorder may be accompanied by depression, substance abuse, or one or more other anxiety disorders. In severe cases, the person may have trouble working or socializing.

### Major Depressive Disorder

Major depressive disorder refers to a class of syndromes characterized by negative affect and repeated episodes of depression without any history of independent episodes of mood elevation and over-activity that fulfill the criteria of mania. Multiple subtypes of major depressive disorders are recognized, including these with atypical characteristics, psychotic components, etc. The age of onset and the severity, duration and frequency of the episodes of depression are all highly variable. The disorder may begin at any age. The symptoms of major depressive disorder typically develop over days to weeks. Prodromal symptoms include generalized anxiety, panic attacks, phobias or depressive symptoms and may occur during several months preceding the episode. Individual episodes also last between 3 and 12 months but recur less frequently. Most patients are asymptomatic between episodes, but a minority of patients may develop a persistent depression, mainly in old age. Individual episodes of any severity are often precipitated by stressful life events. Common symptoms of a depressive episode include reduced concentration and attention; reduced self-esteem and self-confidence; ideas of guilt and unworthiness, ideas or acts of self-harm or suicide; disturbed sleep; and diminished appetite. In certain embodiments, a major depressive episode follows a psychosocial stressor, e.g., death of a loved one, marital separation, childbirth or the end of an important relationship.

The lowered mood varies little from day to day and is often unresponsive to circumstances, yet may show a characteristic diurnal variation as the day goes on. As with manic episodes, the clinical presentation shows marked individual variations, and atypical presentations are particularly common in adolescence. In some cases, anxiety, distress, and motor agitation may be more prominent at times that the depression, and the mood change may also be masked by added features such as irritability, excessive consumption of alcohol, histrionic behavior, and exacerbation of pre-existing phobic or obsessional symptoms, or by hypochondria.

### Psychiatric Evaluations

In certain embodiments, the effects or efficacy of treatment with the present agent/composition are evaluated by the subject and/or a medical professional, e.g., the subject's physician. In certain embodiments, the evaluation is conducted within about 10 minutes, within about 15 minutes, within about 20 minutes, within about 25 minutes, within about 0.5 hours, within about 1 hour, within about 2 hours, within about 2.5 hours, within about 3 hours, within about 3.5 hours, within about 4 hours, within about 4.5 hours, within about 5 hours, within about 5.5 hours, within about 6 hours, within about 6.5 hours, within about 7 hours, within about 7.5 hours, within about 8 hours, within about 8.5 hours, within about 9 hours, within about 9.5 hours, within about 10 hours, within about 10.5 hours, within about 11 hours, within about 11.5 hours, within about 12 hours, within about 18 hours, within about 1 day, within about 2 days, within about 3 days, within about 4 days, within about 5 days, within about 6 days, within about 1 week, within about 2 weeks, within about 3 weeks, within about 4 weeks, within about 1 month, within about 2 months, within about 3 months, within about 4 months, within about 5 months, within about 6 months, within about 1 year, within about 2 years, or longer, following a stressor and/or administration of the present agent/composition.

Psychiatric evaluations of a patient being treated with the present method can be conducted to determine whether the method is effective. In certain embodiments, the psychiatric evaluation may be carried out before treatment, at the time of treatment, during treatment, and/or after treatment. When the psychiatric evaluation is carried out both before treatment and after (and/or during) treatment with the present method, the results of the evaluation before treatment can provide a baseline for comparison to the results of the evaluation during and/or after treatment. In certain embodiments, psychiatric evaluation is conducted only after treatment.

Psychophysiological stress tests can be performed to measure the amount of stress-induced anxiety present in the various systems of the body (i.e. muscular, cardiovascular, digestive, respiratory and neurological systems). These stress tests are routinely used in the art. Test results are compared to both local and national norms, to determine if the individual is exhibiting an excessive amount of physiological anxiety and whether or not they are able to recover from a standardized stressful stimuli in an appropriate length of time.

Psychiatric testing can be used to monitor a subject to determine the emotional and/or social etiology of the stress disorder. These tests are known in the art and include health-related assessments, mental health assessments, personality tests, and personality type assessment.

In certain embodiments, clinician-administered evaluation and/or self-report instruments are used, with the aim of measuring baseline symptomatology as well as drug actions on (1) the overall severity of the disorder, (2) the core symptoms, and (3) depressed mood.

Non-limiting examples of psychiatric evaluation tools and questionnaires include the following measures.

The Diagnostic and Statistical Manual of Mental Disorders (DSM-5) includes the revised diagnostic criteria for PTSD. See, American Psychiatric Association: Diagnostic and Statistical Manual of Mental Disorders, Fifth Edition. Arlington, Va., American Psychiatric Association, 2013. See also ptsd.va.gov/professional/PTSD-verview/dsm5_criteria_ptsd.asp.

The Structured Clinical Interview for DSM-IV Axis I Disorders, Patient Edition (SCID-P) is a semi-structured interview that provides probe questions as well as follow-up questions to be asked by the clinician to assist in diagnosis. First et al., Structured Clinical Interview for DSM-IV TR Axis I Disorders, Research Version, Patient Edition (SCID-I/P). New York: New York State Psychiatric Institute, Biometrics Research; 2001. It includes an overview to obtain information about demographics, work, chief complaint, history of present illness, past history, treatment history, and current functioning. The main body of SCID-P includes 9 modules that are designed to diagnose 51 mental illnesses in all.

The SCID-P for DSM-5 is the SCID—Patient version, and is the next edition of the SCID modified to incorporate the new DSM-5 criteria.

The Clinician-Administered PTSD Scale (CAPS) is a structured clinical interview designed to assess the essential features of PTSD as defined by the DSM-IV. Weathers et al., Clinician-administered PTSD scale: a review of the first ten years of research. Depress Anxiety. 2001; 13(3):132-156. The CAPS can be used to provide categorical ratings of diagnostic status as well as a quantitative index of symptom severity. Both frequency and intensity scores are derived for each individual symptom. The CAPS total score is based on an individual's response to the 17 items that assess the frequency and intensity of current PTSD symptoms. Subscales of the CAPS are utilized to assess specific symptom clusters. The total score can range from 0 to 136.

The Clinician-Administered PTSD Scale for DSM-5 (CAPS-5) is a 30-item structured interview that can be used to make current (past month) diagnosis of PTSD, make lifetime diagnosis of PTSD, and to assess PTSD symptoms over the past week. CAPS-5 is a 30-item questionnaire, corresponding to the DSM-5 diagnosis for PTSD. The language of the CAPS-5 reflects both changes to existing symptoms and the addition of new symptoms in DSM-5. Weathers, F. W., et al (2013). The Clinician-Administered PTSD Scale for DSM-5 (CAPS-5).

The Treatment Outcome PTSD Scale (TOP-8) is a brief interviewer-administered scale designed specifically for the assessment of commonly occurring signs and symptoms of PTSD that are subject to change in response to treatment (Davidson, J. R., & Colket, J. T. (1997). The eight-item treatment-outcome post-traumatic stress disorder scale: A brief measure to assess treatment outcome in post-traumatic stress disorder. International Clinical Psychopharmacology, 12(1), 41-44). The TOP-8 is comprised of eight items, each measured on a scale of 0-4, with defined anchors given for each item. The items are representative of the three core features of PTSD with a maximum possible score of 32.

The Hamilton Psychiatric Rating Scale for Anxiety (HAM-A) is a widely used observational rating measure of anxiety severity. The scale consists of 14 items. Each item is rated on a scale of 0 to 4. This scale is administered to assess the severity of anxiety and its improvement during the course of treatment. The HAM-A total score is the sum of the 14 items and the score ranges from 0 to 56. Hamilton M. The Assessment of Anxiety-States by Rating. Br J Med Psychol. 1959; 32(1):50-55.

The Montgomery-Asberg Depression Rating Scale (MADRS) is a 10-item instrument used for the evaluation of depressive symptoms in adults and for the assessment of any changes to those symptoms. Montgomery S. A., et al., A new depression scale designed to be sensitive to change. Br J Psychiatry. 1979 April; 134:382-389. Each of the 10 items is rated on a scale of 0 to 6, with differing descriptors for each item. These individual item scores are added together to form a total score, which can range between 0 and 60 points.

The Young Mania Rating Scale, item 1 (YMRS-1) used to assess mood elevation on the infusion days. Young R C, et al. Rating-Scale for Mania—Reliability, Validity and Sensitivity. Br J Psychiatry. 1978; 133 (NOV):429-435.

The Brief Psychiatric Rating Scale (BPRS) is used to assess acute behavioral changes during the infusions. Overall J E et al., The Brief Psychiatric Rating-Scale. Psychol. Rep. 1962; 10(3):799-812 Four key BPRS items for the positive (+) symptoms of psychosis are used: conceptual disorganization, hallucinatory behavior, suspiciousness, and unusual thought content. Three items representing the negative (−) symptoms of psychosis will also be used: blunted affect, emotional withdrawal, and motor retardation.

The Clinician-Administered Dissociative States Scale (CADSS) is used to measure dissociative effects during the infusions. Bremner J D, et al., Measurement of Dissociative States with the Clinician-Administered Dissociative States Scale (CADSS). J Trauma Stress. 1998; 11(1):125-136 The scale includes 19 questions and 8 observer ratings scored from 0 (not at all) to 4 (extremely). The CADSS measures impairment in body perception, environmental perception, time perception, memory impairment, and feelings of unreality.

The Patient Rating Inventory of Side Effects (PRISE) is a patient self-report used to qualify side effects by identifying and evaluating the tolerability of each symptom. Levine J, Schooler N R. SAFTEE: A technique for the systematic assessment of side effects in clinical trials. Psychopharmacol Bull. 1986; 22(2):343-381.

The Clinical Global Impression (CGI) scale assesses treatment response in psychiatric patients. The administration time is 2 minutes. This scale consists of three items: Severity of Illness (item 1); Global Improvement (item 2); and Efficacy Index (item 3). Item 1 is rated on a seven-point scale (1=normal, 7=among the most extremely ill patients) as is item 2 (1=very much improved, 7=very much worse). Each includes an additional response of “not assessed.” Item 3 is rated on a four-point scale (from “none” to “outweighs therapeutic effect”).

The Impact of Events Scale (IES) is one of the most widely used self-report measures of stress reactions to traumatic events. Horowitz et al., Impact of Event Scale: a measure of subjective stress. Psychosom Med. 1979 May; 41(3):209-218. See also, Weiss et al., The Impact of Event Scale—Revised In: Wilson J, Keane T M, eds. Assessing psychological trauma and PTSD. New York: Guilford; 1996:399-411. It measures both intrusion and avoidance. Sundin et al., Impact of Event Scale: psychometric properties. Br J Psychiatry. 2002 March; 180:205-209. Joseph S. Psychometric evaluation of Horowitz's Impact of Event Scale: a review. J Trauma Stress. 2000 January; 13(1):101-113. The total score can range from 0 to 75.

The Posttraumatic Stress Disorder Checklist (PCL-5) is a 17-item self-report measure reflecting DSM-5 symptoms of PTSD. The PCL-5 measures symptoms in response to stressful situations (Weathers, F., et al. (1993). The PTSD checklist (PCL): Reliability, validity, and diagnostic utility. Annual Convention of the International Society for Traumatic Stress Studies, San Antonio, Tex.).

The Quick Inventory of Depressive Symptomatology, Self Report (QIDS-SR) is a 16-item self-rated instrument designed to assess the severity of depressive symptoms present in the past seven days. Rush A J, Trivedi M H, Ibrahim H M et al. The 16-Item quick inventory of depressive symptomatology (QIDS), clinician rating (QIDS-C), and self-report (QIDS-SR): a psychometric evaluation in patients with chronic major depression. Biol. Psychiatry. 2003; 54(5):573-583. The 16 items cover the nine symptom domains of major depression, and are rated on a scale of 0-3. Total score ranges from 0 to 27, with ranges of 0-5 (normal), 6-10 (mild), 11-15 (moderate), 16-20 (moderate to severe), and 21+(severe).

The Childhood Trauma Questionnaire (CTQ) is a 28-item self-report instrument that assesses childhood trauma in the following areas: physical, sexual and emotional abuse and physical and emotional neglect. Bernstein D P, Stein J A, Newcomb M D et al. Development and validation of a brief screening version of the Childhood Trauma Questionnaire. Child Abuse Negl. 2003 February; 27(2):169-190. Each item is rated on a scale of 1 (never true) to 5 (very often true). The 5 subscales are then totaled, with scores ranging from 5-25 for each traumatic category.

Visual Analogue Scales (VAS) are used to assess subjective state changes. Bond A, Lader M. The use of analogue scales in rating subjective feelings. Br J Med Psychol. 1974; 47(3):211-218. They are 100-mm horizontal lines marked proportionately to the perceived intensity of the subjective experience (0=not at all, to 10=extremely) for the following states: anxious, depressed, drowsy, high, hungry, and nauseous.

The Sheehan Disability Scale (SDS) is a self-report disability measure. It has demonstrated sensitivity to impairment and changes as a result of treatment across a wide range of psychiatric disorders. The SDS asks only about current levels of impairment, providing no indication of whether the person has done better or worse in the past, thus making it a reasonable short-term outcome measure that is un-confounded by historical impressions. The dependent variable is the total score, which is based on the sum of three 10-point items (work, social life, and family life), with higher scores reflecting greater disability. Sheehan D. The Anxiety Disease. New York, N.Y.: Scribner; 1983.

The Wechsler Abbreviated Scale of Intelligence 2-Subtest (WASI-2) is a reliable brief measure of IQ for 6 to 89 year-olds that includes Vocabulary (an estimate of verbal fluid abilities) and Matrix Reasoning (an estimate of nonverbal fluid abilities). Wechsler D. Wechsler Abbreviated Scale of Intelligence San Antonio, Tex.: Psychological Corporation; 1999. It is extensively used in clinical, educational, and research settings. Average reliability coefficient is 0.96 and test-retest reliability is 0.88.

The Hopkins Verbal Learning Test (HVLT) is a repeatable test of memory acquisition and delayed recall of words. Subjects are presented with the same 12-item list for 3 learning trials and asked each time to repeat the items on each list. Delayed recall and recognition conditions are administered later. Dependent variables used in this study include total learning over the 3 trials (for the acquisition variable) and total delayed recall score (for the recall component). Brandt J, Benedict R. Hopkins Verbal Learning Test, Revised. Odessa, Fla.: Psychological Assessment Resources; 1997.

The Profile of Mood States-Bipolar (POMS-Bi) scale measures moods and feelings primarily in clinical rather than nonclinical settings. It can help to determine an individual's psychiatric status for therapy, or be used to compare mood profiles associated with various personality disorders. It is also a useful instrument in identifying the effects of drug treatments.

The Post-Traumatic Cognitions Inventory (PTCI) is a 33-item scale, which is rated on a Likert-type scale ranging from 1 (totally disagree) to 7 (totally agree). Scale scores are formed for the three subscales, which show a high degree of intercorrelation (rs=0.57-0.75).

The New Cognitions scale is a 6-item pilot scale, which is rated on a Likert-type scale ranging from 1 (not at all) to 4 (a lot). The scale is based on the Post Traumatic Growth Inventory (PTGI) from which items have been directly selected (new items were added to the scale as well), and on the Brief-COPE (see Carver, C. S. (1997) “You want to measure coping but your protocol's too long: Consider the brief COPE.” International Journal of Behavioral Medicine 4; 92-100).

The Medical Outcomes Study (MOS) Social Support Survey is a 19-item self-report measure designed to assess levels of functional social support. The MOS-SS has two subscales (emotional and instrumental social support) to identify potential social support deficits (Sherbourne, C. D. & Stewart, A. L. (1991). “The MOS Social Support Survey.” Soc Sci Med 32(6): 705-714).

The Purpose in Life test-Short Form (PIL-SF) is a brief, 4-item form of the 20-item Purpose in Life test. This scale asks respondents to report to what extent they have achieved their goals in life, and to what extent they perceive their life to be meaningful or purposeful. (Schulenberg et al 2010; Psychotherapy (Chic). 2008 December; 45(4):447-63).

Posttraumatic Growth Inventory (PTGI)-Short Version is a 10-item shortened version of the PTGI self-report questionnaire (ref). It asks respondents to rate the extent to which they have changed as the result of experiencing a highly stressful life event. Items span positive changes in five domains: relating to others, new possibilities, personal strength, spiritual change, and appreciation of life (Cann, A., et al. (2010). A short form of the Posttraumatic Growth Inventory. Anxiety, Stress & Coping, 23, 127-137).

The Quality of Life Enjoyment and Satisfaction Questionnaire (Q-LES-Q) is a self-report scale measuring the degree of enjoyment and satisfaction experienced by subjects in various areas of daily functioning. The summary scores are reliable and valid measures of these dimensions in a group of depressed subjects (Endicott J, et al. Quality of Life Enjoyment and Satisfaction Questionnaire: A New Measure. Psychopharmacology Bulletin; 1993; 29:321-326).

In certain embodiments, self-evaluation of the subject being treated is conducted.

### Pharmaceutical Compositions

While it is possible that the present agent may be administered as the raw chemical, it is possible to present the active ingredient as a pharmaceutical composition. Accordingly, the disclosure further provides a pharmaceutical composition, which comprises the present compound and/or salts, solvates and physiological functional derivatives thereof, and one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical composition including admixing the present compound, or salts, solvates and physiological functional derivatives thereof, with one or more pharmaceutically acceptable carriers, diluents or excipients.

The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing compound 20, and pharmaceutically acceptable excipients.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

Pharmaceutical compositions of the present invention may be presented in unit dose forms containing a predetermined amount of active ingredient per unit dose. Such a unit may contain, for example, 5 μg to 1 g, preferably 1 mg to 700 mg, more preferably 5 mg to 100 mg of the present compound, depending on the condition being treated, the route of administration and the age, weight and condition of the patient. Such unit doses may therefore be administered more than once a day. Preferred unit dosage compositions are those containing a daily dose or sub-dose (for administration more than once a day), as herein above recited, or an appropriate fraction thereof, of an active ingredient. Furthermore, such pharmaceutical compositions may be prepared by any of the methods well known in the pharmacy art.

Pharmaceutical compositions of the present invention may be adapted for administration by any appropriate route, for example by the oral (including buccal or sublingual), transdermal, inhaled, nasal, ocular, intraperitoneal, intravenous, intracerebroventricular, intracisternal injection or infusion, subcutaneous, implant, sublingual, subcutaneous, intramuscular, rectal, mucosal, ophthalmic, intrathecal, intra-articular, intra-arterial, sub-arachinoid, bronchial and lymphatic administration, or parenteral (including intravenous and intramuscular) route. The present composition may be injected. Such compositions may be prepared by any method known in the art of pharmacy, for example by bringing into association the active ingredient with the carrier(s) or excipient(s). The present composition may be administered parenterally or systemically. The present agent/composition may be delivered to the brain using a formulation capable of delivering a therapeutic agent across the blood brain barrier.

In a further embodiment, the present invention provides a pharmaceutical composition adapted for administration by the oral route, the treatment of stress-induced affective disorder.

Pharmaceutical compositions of the present invention which are adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

It should be understood that, in addition to the ingredients particularly mentioned above, the compositions may include other agents conventional in the art having regard to the type of formulation in question, for example those suitable for oral administration may include flavoring agents.

A therapeutically effective amount of a compound of the present invention will depend upon a number of factors including, for example, the age and weight of the subject, the precise condition requiring treatment and its severity, the nature of the formulation, and the route of administration, and will ultimately be at the discretion of the attendant physician or veterinarian.

### Kits

Also provided are kits for use in the present methods of prophylactically treating, or treating, a stress-induced affective disorder.

The kits can include an agent or composition provided herein, and instructions providing information to a health care provider regarding usage in accordance with the present methods. The kit may optionally contain a second agent or composition. Instructions may be provided in printed form or in the form of an electronic medium such as a floppy disc, CD, or DVD, or in the form of a website address where such instructions may be obtained. A unit dose of a compound or composition provided herein, or a second agent or composition, can include a dosage such that when administered to a subject, a therapeutically or prophylactically effective plasma level of the compound or composition can be maintained in the subject for at least 1 days. In some embodiments, a compound or composition can be included as a sterile aqueous pharmaceutical composition or dry powder (e.g., lyophilized) composition. In some embodiments, suitable packaging is provided. As used herein, “packaging” includes a solid matrix or material customarily used in a system and capable of holding within fixed limits a compound provided herein and/or a second agent suitable for administration to a subject. Such materials include glass and plastic (e.g., polyethylene, polypropylene, and polycarbonate) bottles, vials, paper, plastic, and plastic-foil laminated envelopes and the like.

The kits described herein contain one or more containers, which contain compounds, signaling entities, biomolecules and/or particles as described. The kits also contain instructions for mixing, diluting, and/or administrating the compounds. The kits also include other containers with one or more solvents, surfactants, preservative and/or diluents (e.g., saline (0.9% NaCl), or 5% dextrose) as well as containers for mixing, diluting or administering the components to the sample or to the patient in need of such treatment.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are used, the liquid form may be concentrated or ready to use. The solvent will depend on the compound and the mode of use or administration. Suitable solvents for drug compositions are well known and are available in the literature. The solvent will depend on the compound and the mode of use or administration.

The kits comprise a carrier being compartmentalized to receive in close confinement one or more container such as vials, tubes, and the like, each of the container comprising one of the separate elements to be used in the method. For example, one of the container may comprise a positive control in an assay. Additionally, the kit may include containers for other components, for example, buffers useful in the assay.

Unless otherwise indicated, all numbers expressing quantities of ingredients, properties and so forth used in the present disclosure and associated claims are to be understood as being modified in all instances by the term “about.” Accordingly, unless indicated to the contrary, the numerical parameters set forth in this disclosure and attached claims are approximations that may vary depending upon the desired properties sought to be obtained by the examples of the present invention. At the very least, and not as an attempt to limit the application of the doctrine of equivalents to the scope of the claim, each numerical parameter should at least be construed in light of the number of reported significant digits and by applying ordinary rounding techniques. It should be noted that when “about” is at the beginning of a numerical list, “about” modifies each number of the numerical list. Further, in some numerical listings of ranges some lower limits listed may be greater than some upper limits listed. One skilled in the art will recognize that the selected subset will require the selection of an upper limit in excess of the selected lower limit. The term “about” refers to +10% of the referenced value. In other words, the numeric value can be in a range of 90% of the stated value to 110% of the stated value.

This invention will be better understood from the Examples, which follow. However, one skilled in the art will readily appreciate that the specific methods and results discussed are merely illustrative of the invention as described more fully in the claims that follow thereafter.

### Example 1: Fluoroethylnormemantine, a Novel NMDA Receptor Antagonist, for the Prevention and Treatment of Stress-Induced Depression

BACKGROUND: Major depressive disorder (MDD) is a common, recurrent illness affecting millions of people. Recent studies have shown that the N-methyl-D-aspartate (NMDA) receptor is implicated in the pathophysiology of MDD. (R,S)-ketamine, an NMDA receptor antagonist, has been shown to be an effective antidepressant for MDD patients, but has numerous side effects including abuse potential. Here, we characterized a novel NMDA receptor antagonist, fluoroethylnormemantine (FENM), in order to determine its effectiveness as a prophylactic and/or antidepressant against stress-induced depression.
METHODS: A single injection of saline, (R,S)-ketamine (30 mg/kg), or FENM (10, 20, or 30 mg/kg) was administered before or after contextual fear conditioning (CFC) stress in 12956/SvEv mice. Drug efficacy was assayed using a variety of behavioral tests, including the forced swim test (FST), elevated plus maze (EPM), open field (OF), marble burying (MB), and novelty-suppressed feeding (NSF). Neural activity was quantified in the hippocampus. RESULTS: As a prophylactic, FENM attenuated learned fear and decreased stress-induced depressive-like behavior. As an antidepressant, FENM decreased stress-induced depressive-like behavior and facilitated extinction learning. FENM did not alter locomotion or anxiety-like behavior. FENM increased neural activity in the hippocampus.
CONCLUSIONS: Our results indicate that FENM is novel drug that is efficacious both as a prophylactic and as an antidepressant.

Recently, FENM was used as a radiolabeled compound [¹⁸F]-FENM as an innovative PET tracer (33, 34). Here, the authors report that [¹⁸F]-FENM stabilized in the brain 40 minutes post injection with 0.4% of the injected dose being found in the brain. Combined ex vivo autoradiography and immunohistochemical staining demonstrated colocalization of NMDARs and [¹⁸]-FENM, with the highest intensity in the cortex and cerebellum. Most interestingly, if (R,S)-ketamine was administered, [¹⁸]-FENM colocalization with NMDARs was lost, indicating that binding was disabled or blocked. However, although FENM is an NMDAR antagonist with lower affinity than (R,S)-ketamine, it remains unknown whether FENM can be efficacious as a prophylactic or antidepressant.

Here, we sought to characterize FENM as a novel compound for the treatment of stress-induced psychiatric disease. A single injection of saline, (R,S)-ketamine (30 mg/kg), or FENM (10, 20, or 30 mg/kg) was administered before or after contextual fear CFC stress in 12956/SvEv mice. Drug efficacy was assayed using a variety of behavioral tests, including the forced swim test (FST), elevated plus maze (EPM), open field (OF), marble burying (MB), and novelty-suppressed feeding (NSF). Neural activity was quantified in the hippocampus. As a prophylactic, FENM attenuated learned fear and decreased stress-induced depressive-like behavior. As an antidepressant, FENM decreased stress-induced depressive-like behavior and facilitated extinction learning. Our results indicate that FENM is a novel drug that is efficacious both as a prophylactic and as an antidepressant.

### Methods

### Mice

Male 12956/SvEvTac mice were purchased from Taconic (Hudson, N.Y.) at 7 weeks of age. Mice were housed 5 per cage in a 12-h (06:00-18:00) light-dark colony room at 22° C. Food and water were provided ad libitum. Behavioral testing was performed during the light phase. All experiments were approved by the Institutional Animal Care and Use Committee (IACUC) at the New York Psychiatric Institute (NYSPI).

### Drugs

All drugs were resuspended in saline and made fresh for each experiment.

Fluoroethylnormemantine (FENM): FENM was administered in a single dose at 10, 20, or 30 mg/kg of body weight. FENM was generated by M2i (Saint-Cloud, Cedex, France). (R,S)-ketamine (K): (R,S)-ketamine (Ketaset III, Ketamine HCl injection, Fort Dodge Animal Health, Fort Dodge, Iowa) was administered in a single dose at 30 mg/kg of body weight. A dose of 30 mg/kg of body weight was chosen, as previous studies indicated that is the effective dose for prophylactic efficacy (11-13, 35, 36).

### Statistical Analysis

All data were analyzed using Prism (Graphpad Software, La Jolla, Calif.). Alpha was set to 0.05 for all analyses. Generally, the effect of Drug or Group was analyzed using an analysis of variance (ANOVA), using repeated measures where appropriate. Post-hoc Fisher's LSD tests were used where appropriate. All statistical tests and p values are listed in Table 1.

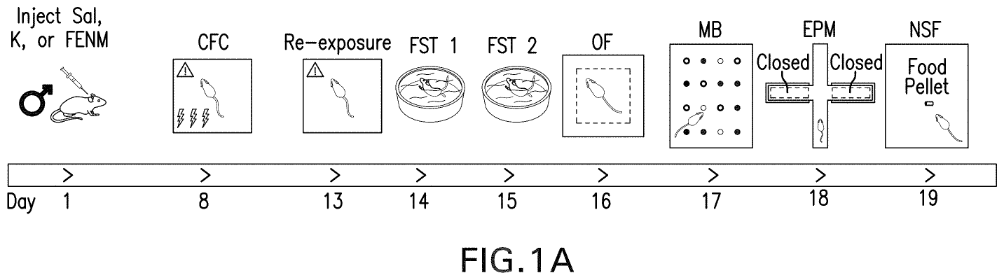

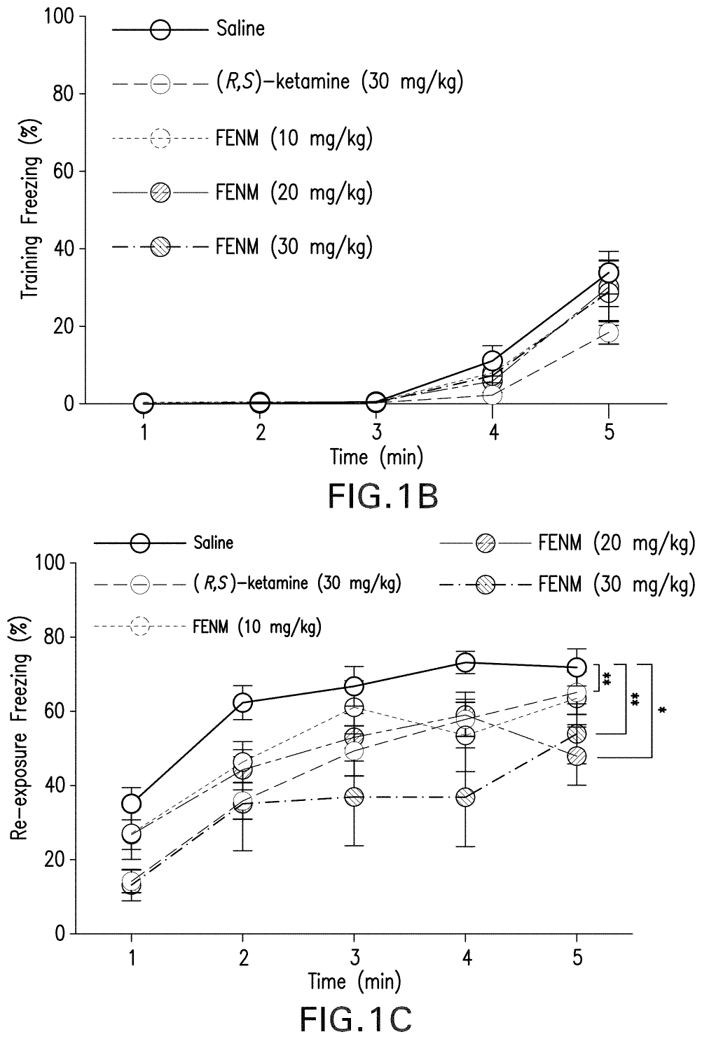

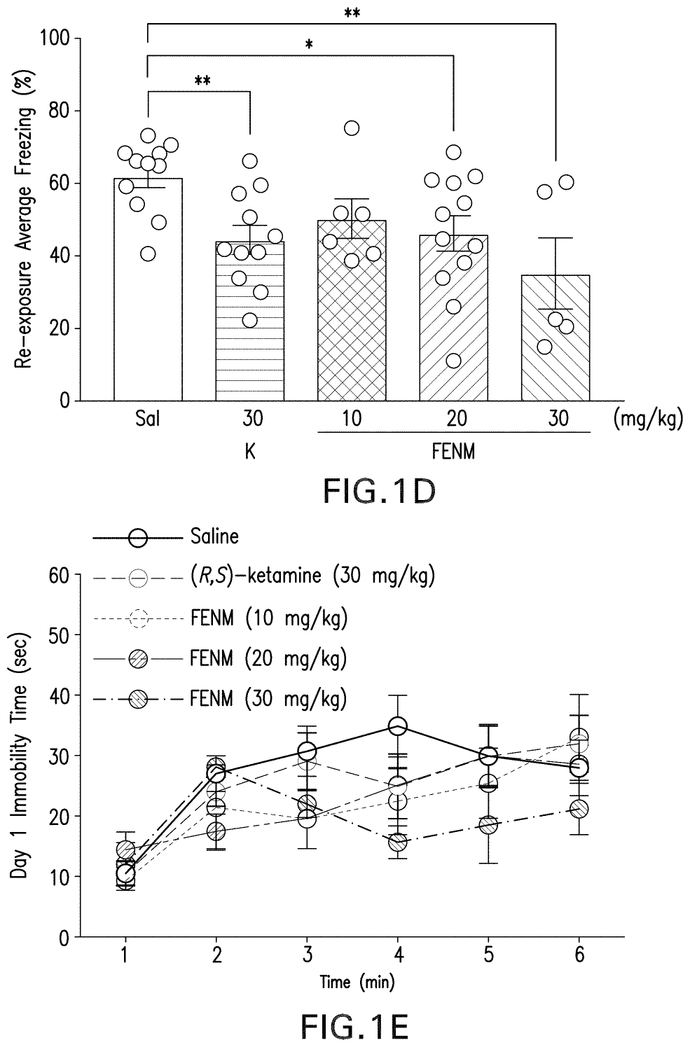

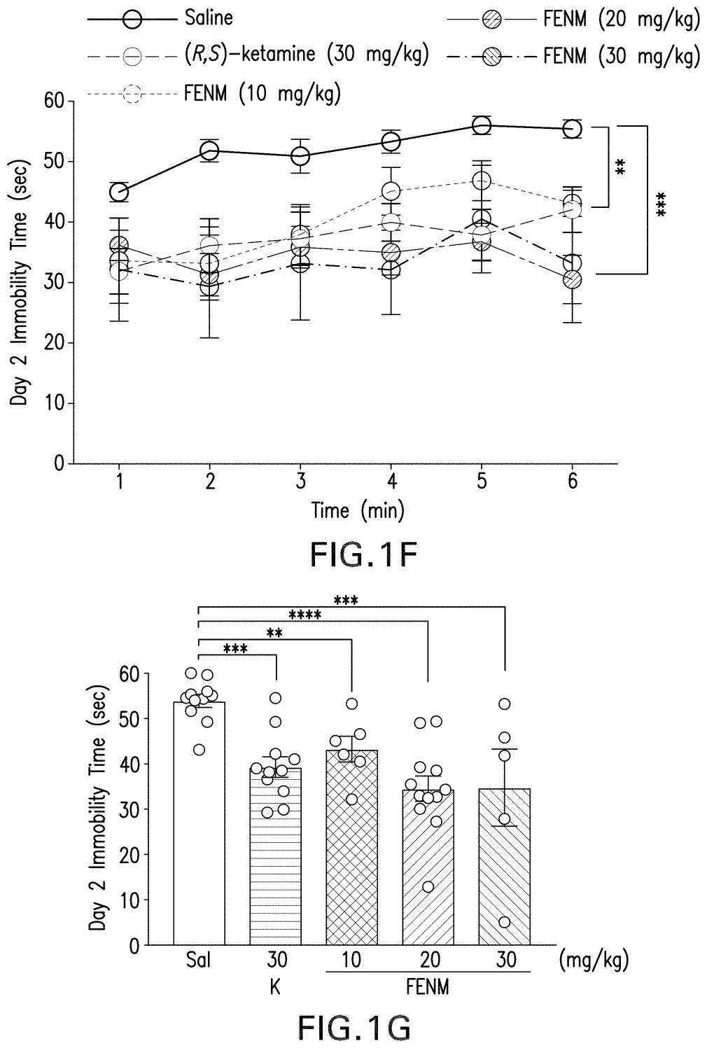

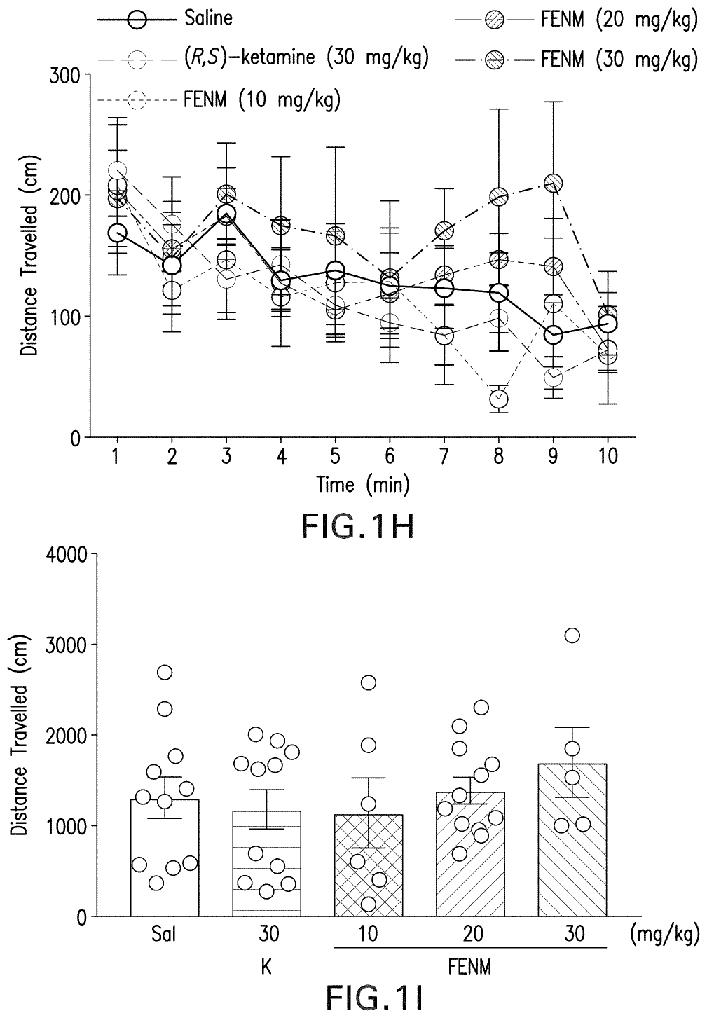

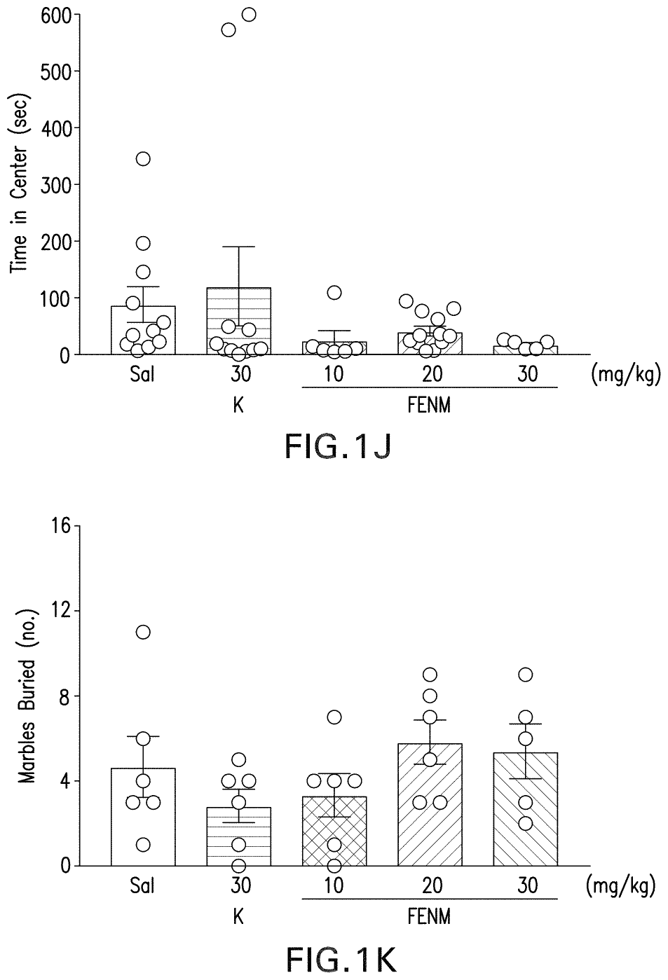

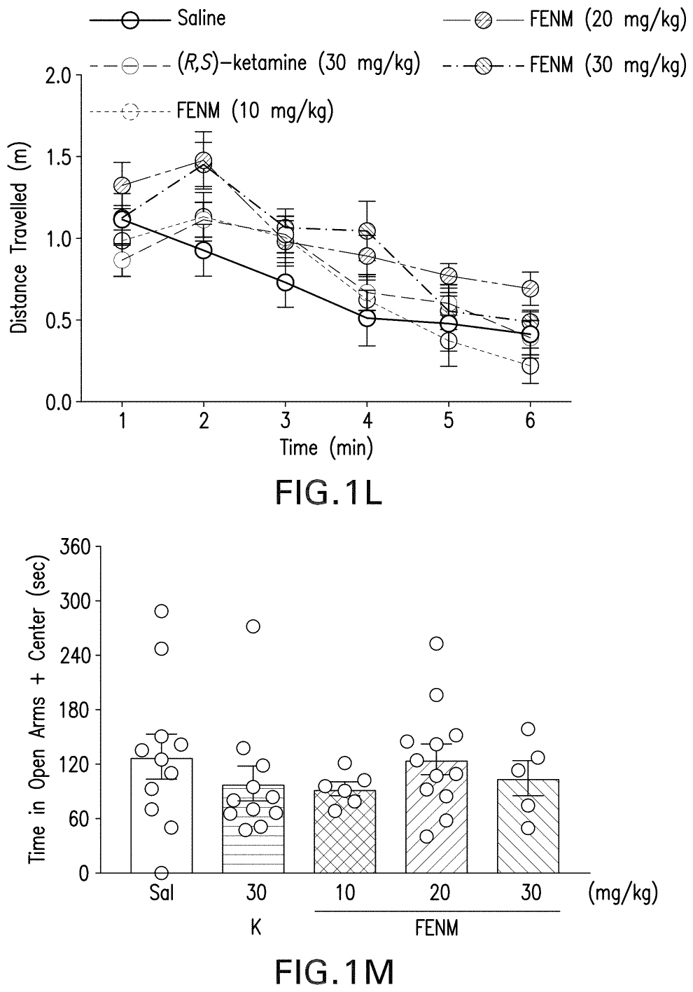

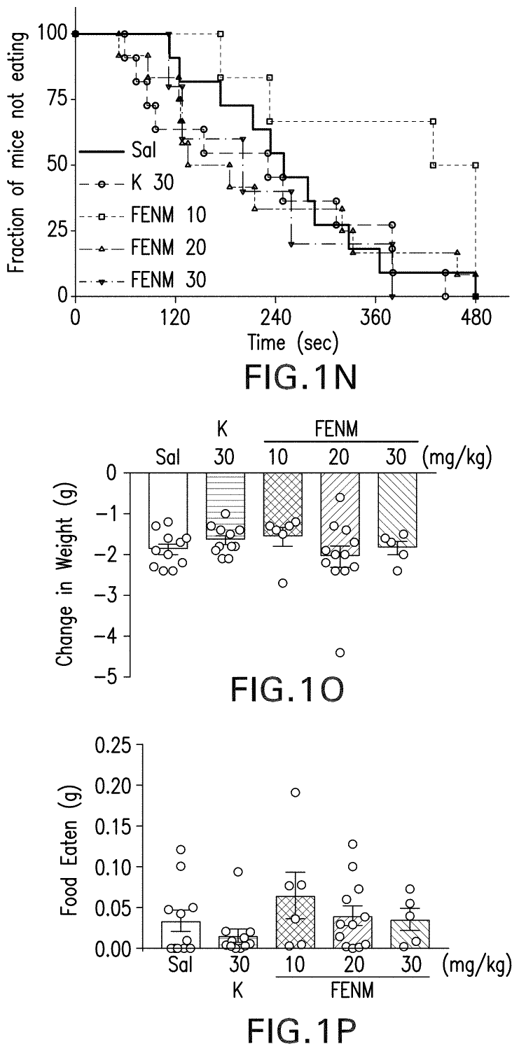

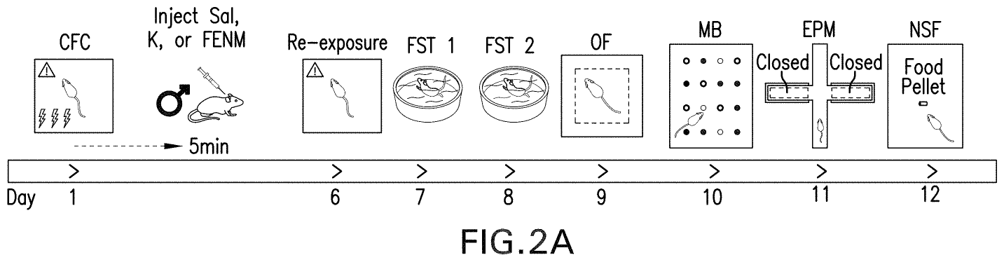

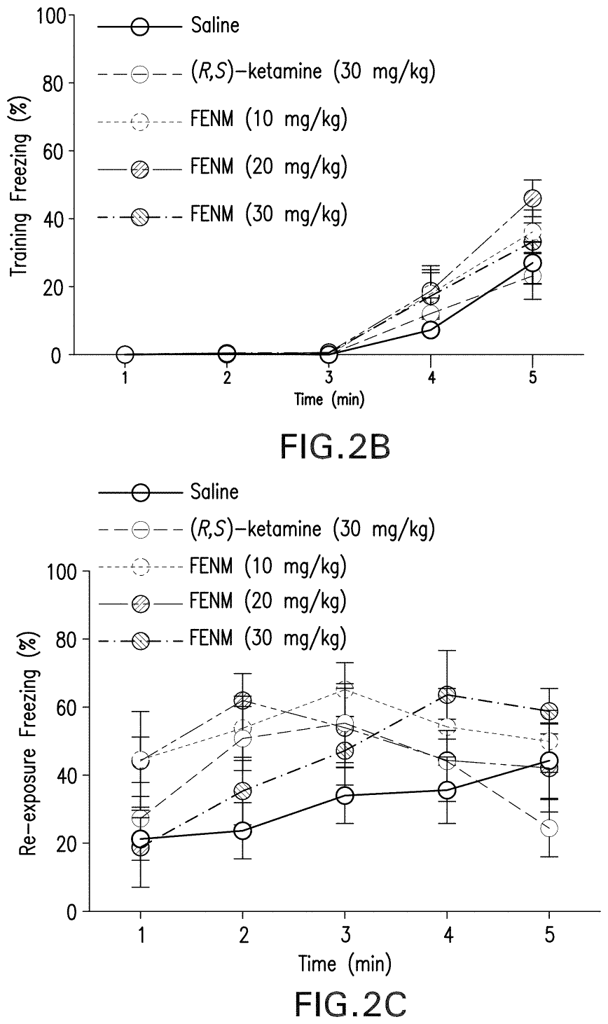

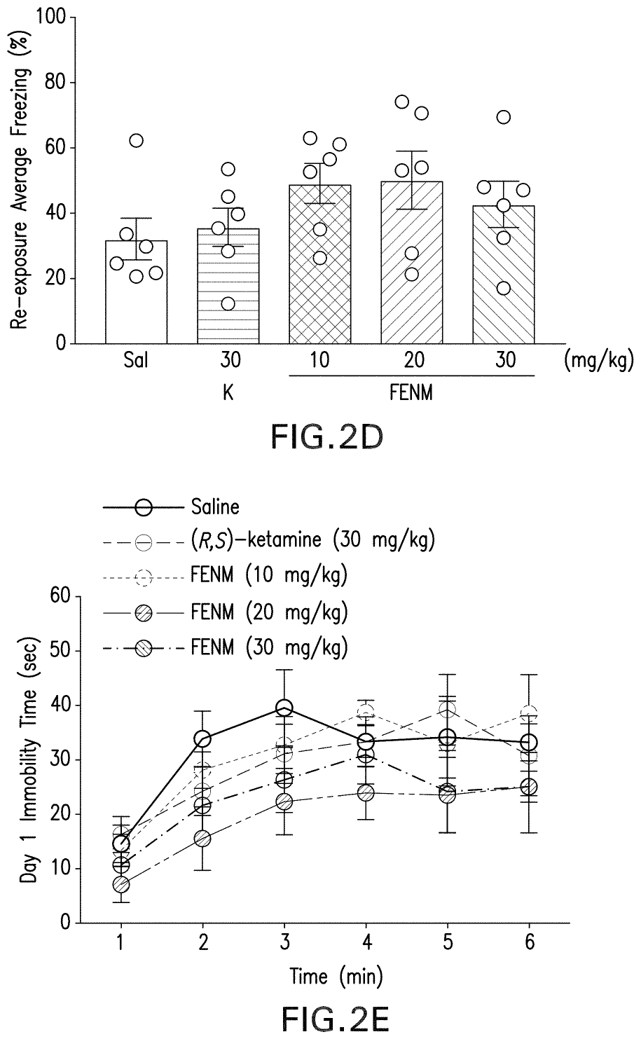

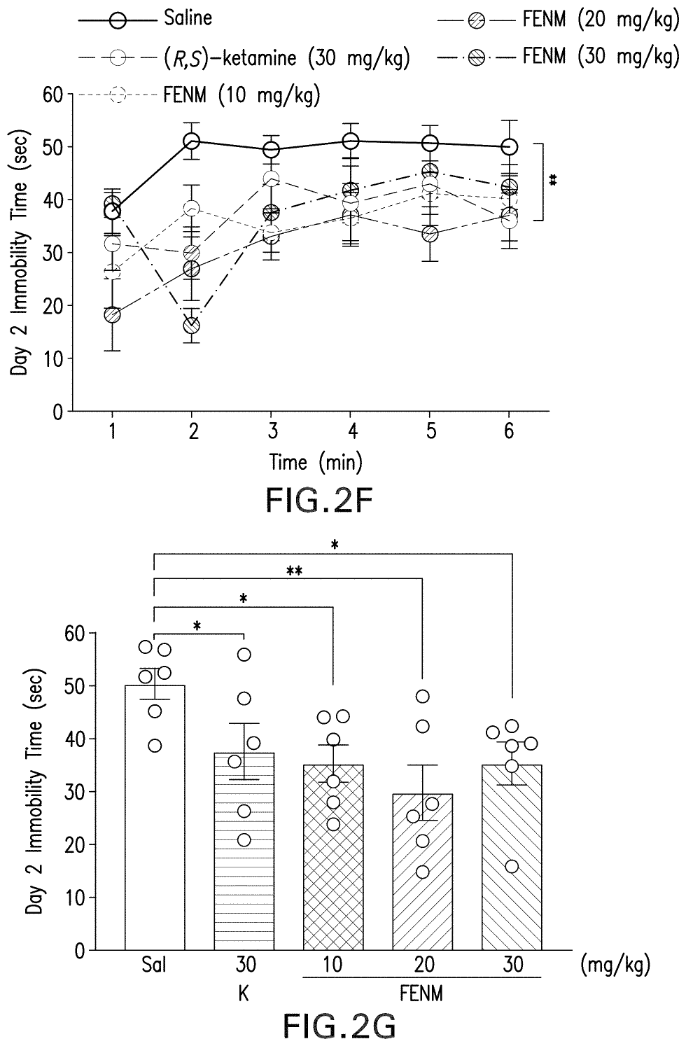

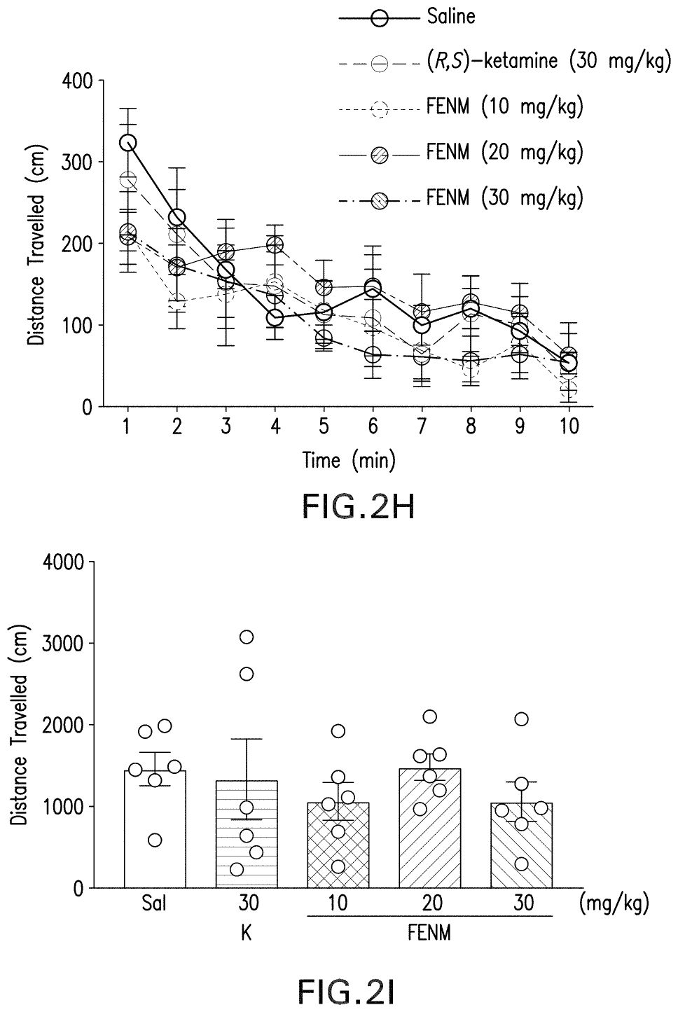

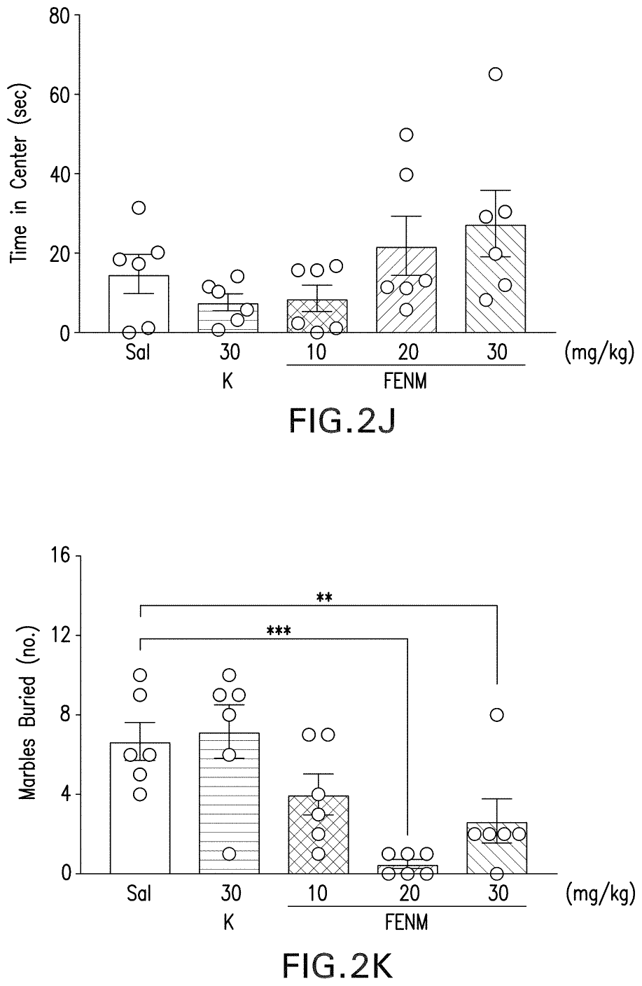

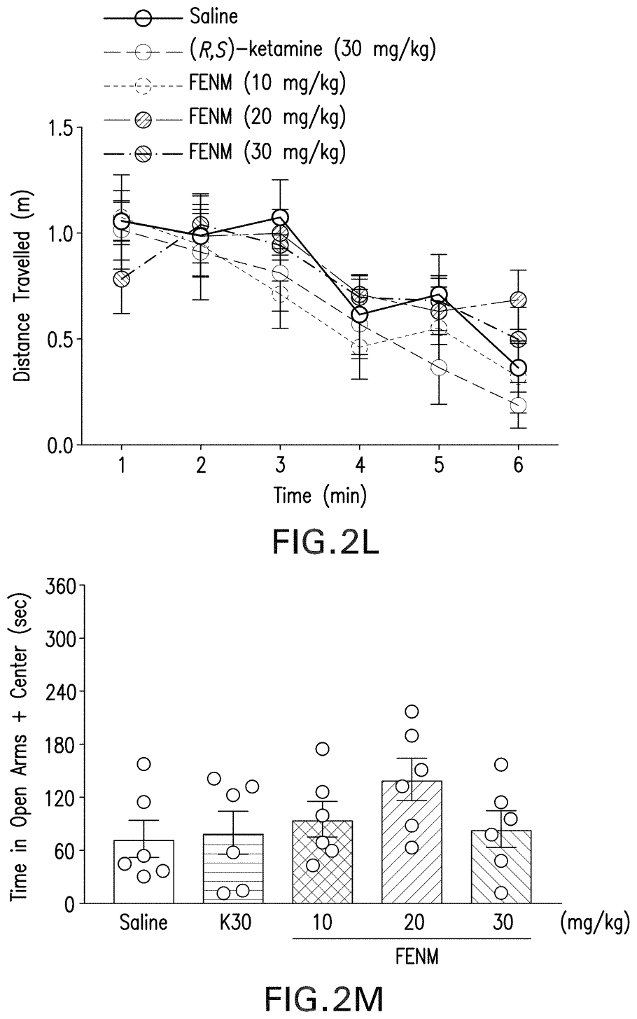

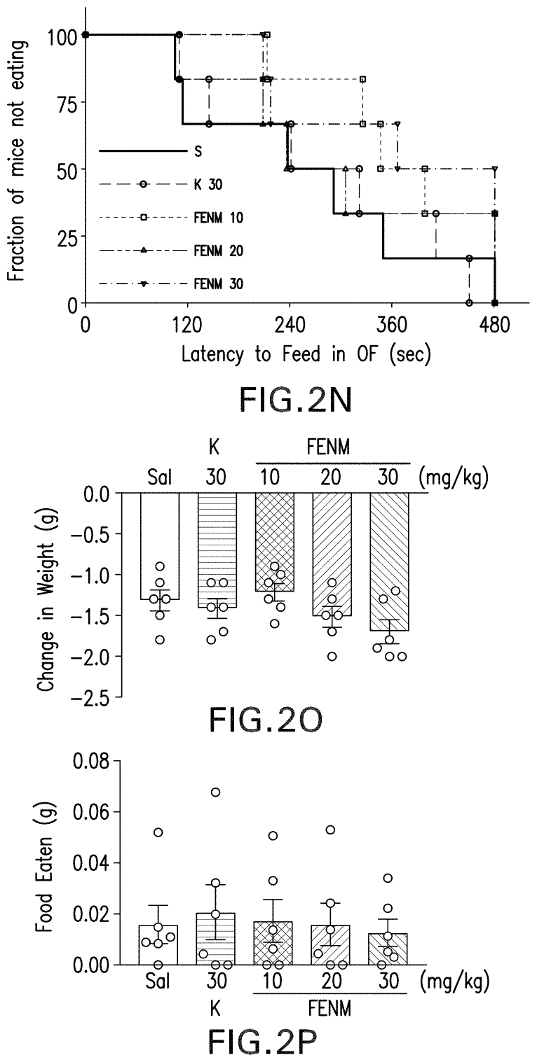

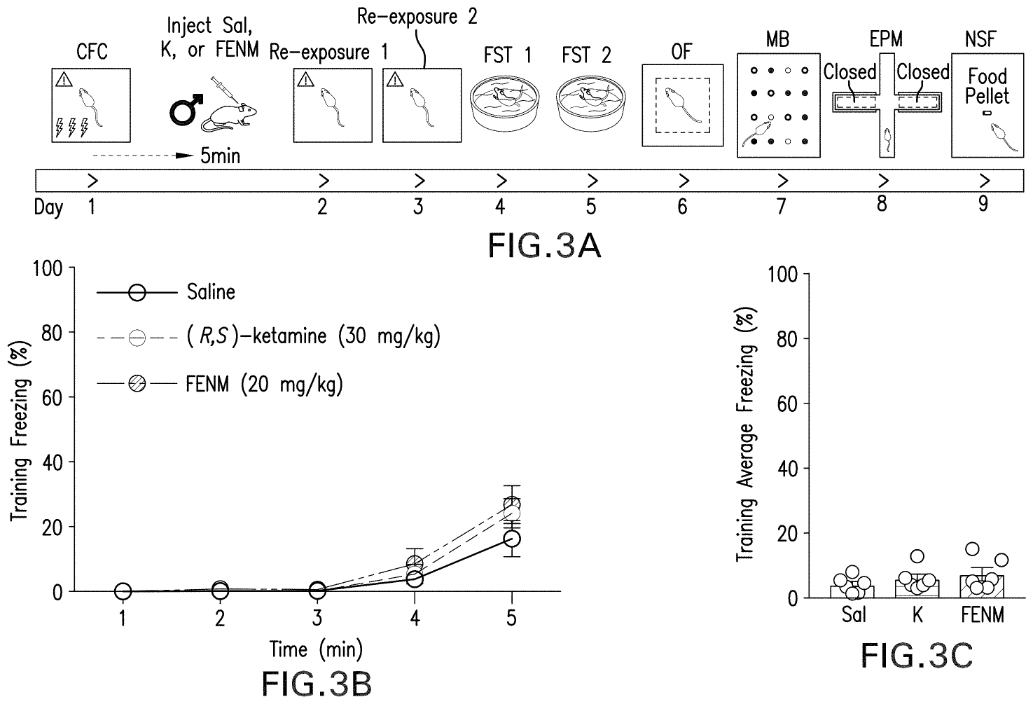

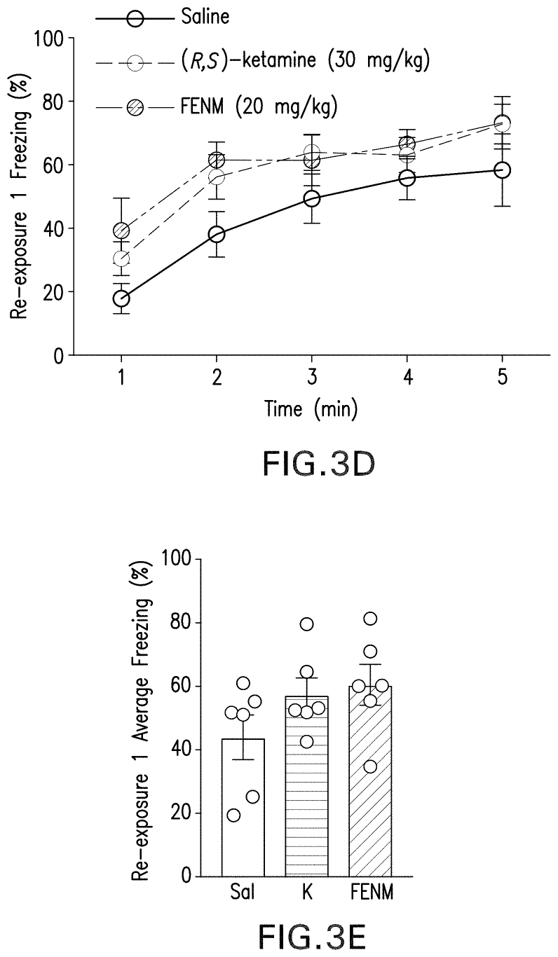

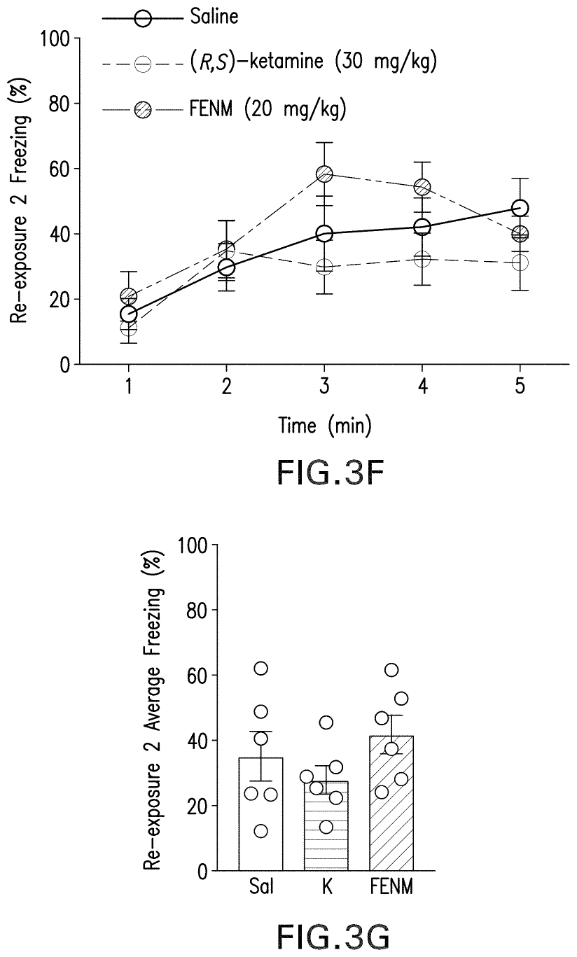

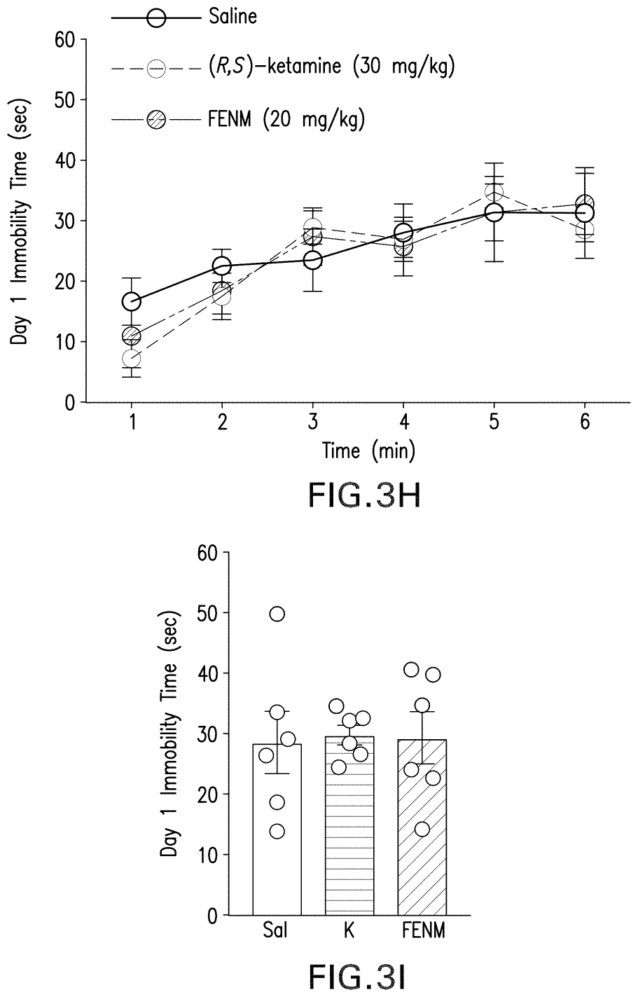

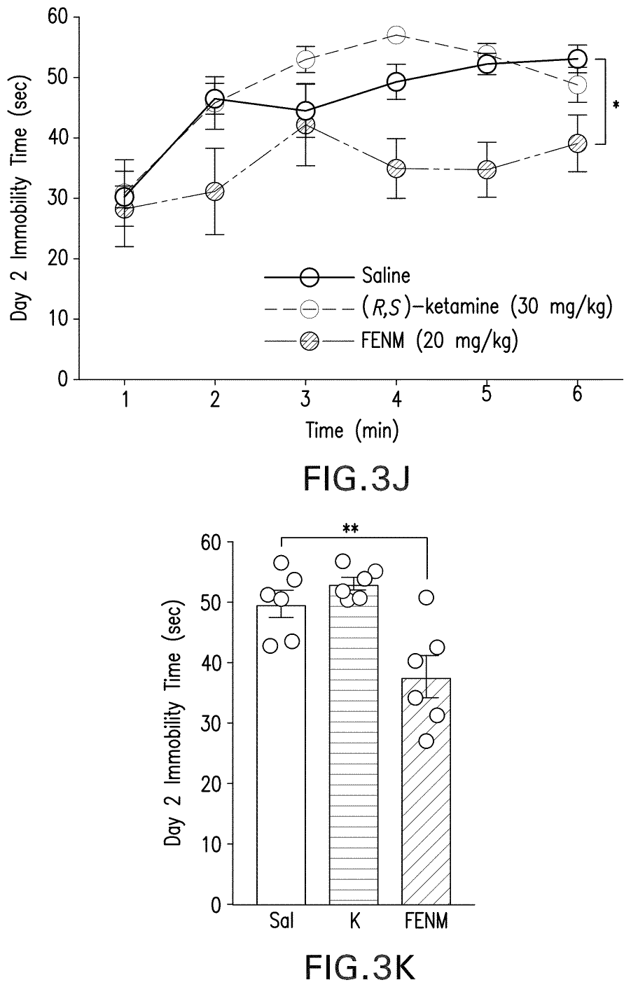

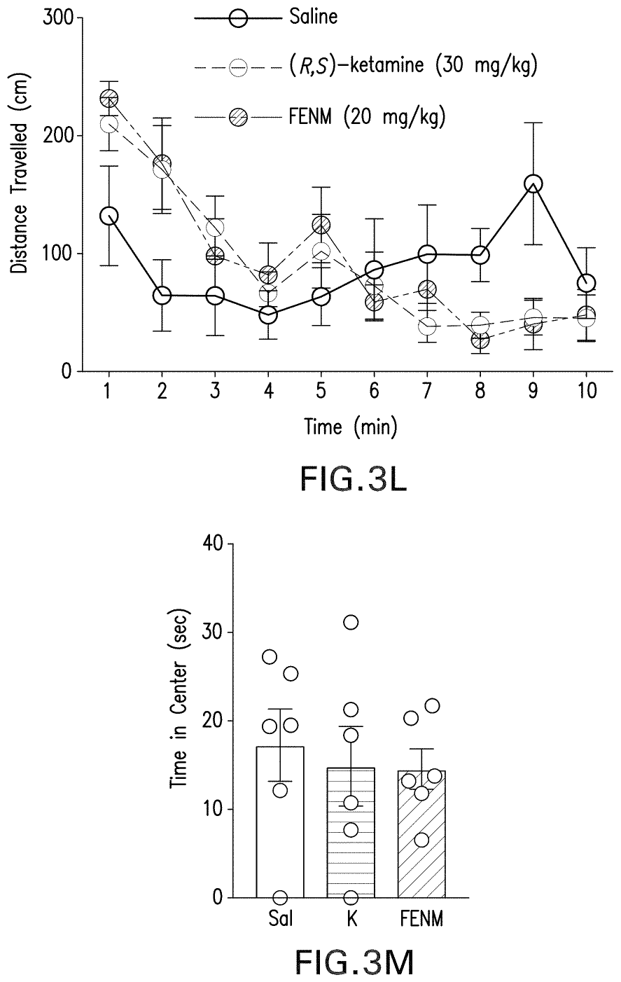

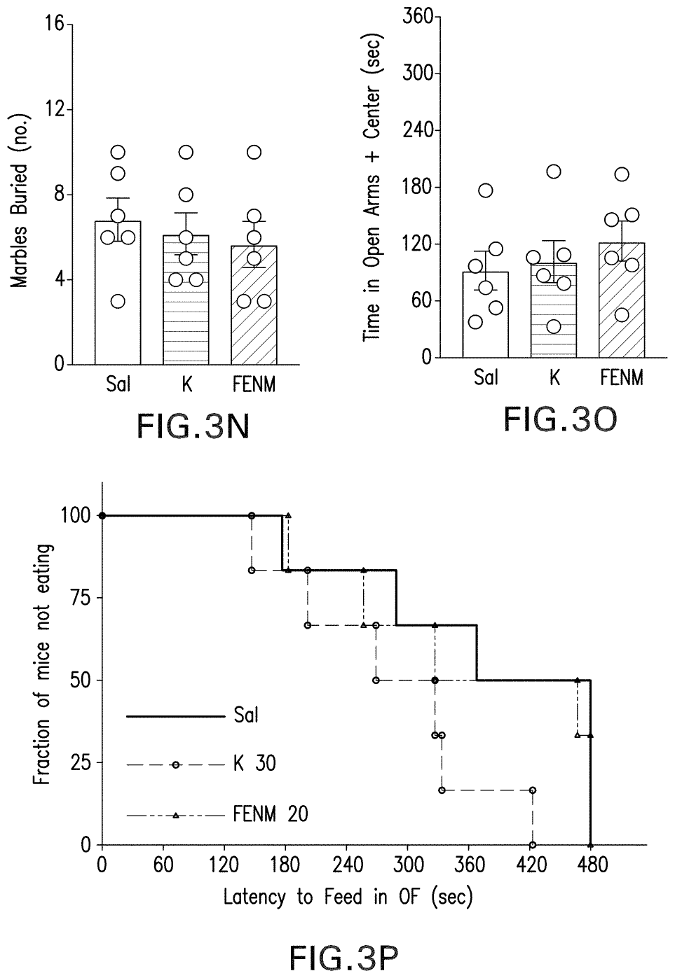

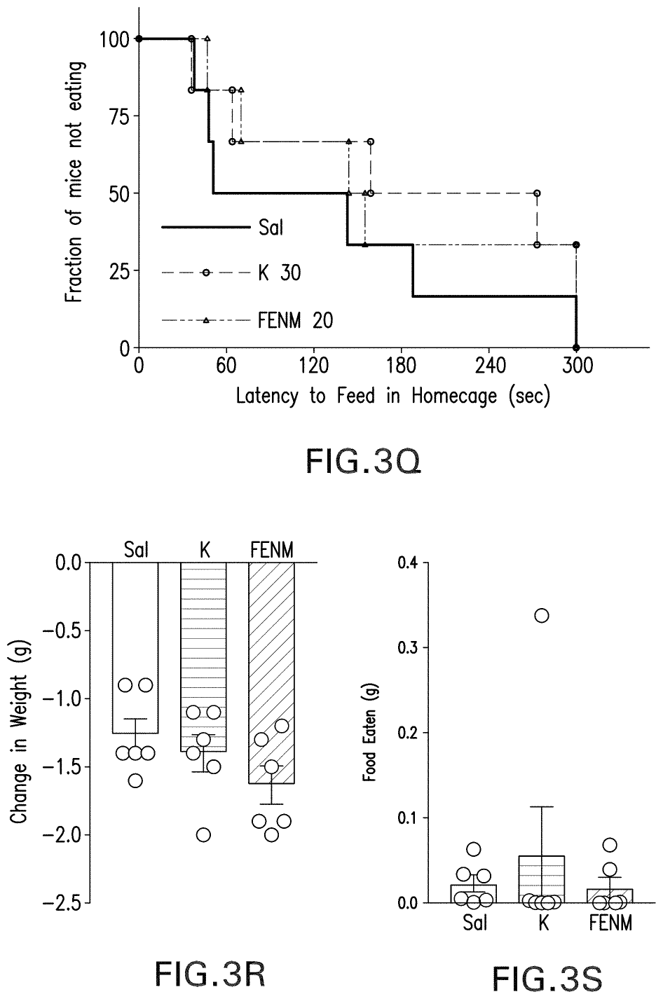

TABLE 1
Statistical analysis

| | Behavioral | | | Statistical | | | ° of | | | |
|---|---|---|---|---|---|---|---|---|---|---|
| Cohort | Paradigm | Abbrev | Measurement | Test | Comparison | F | freedom | p | * | FIG. |
| FIG. 1, | Contextual | CFC | Freezing | RMANOVA | Drug | 1.822 | 4.40 | 0.1435 | — | 1B |
| FENM | Fear | Training | (%) | | Time | 88.65 | 4160 | <0.0001 | *** | |
| 1 week | Conditioning | | | | Drug × Time | 1.181 | 16160 | 0.2882 | — | |
| before | Training | | | | | | | | | |
| stress | Contextual | CFC | Freezing | RMANOVA | Drug | 3.557 | 4.40 | 0.0142 | * | 1C |
| | Fear | Re-exposu | (%) | | Time | 49.730 | 4160 | <0.0001 | *** | |
| | | | | | Drug × Time | 1.732 | 16160 | 0.0456 | * | |
| | | | | Fisher's | Saline vs. | — | — | 0.0086 | ** | |
| | | | | LSD | (R,S)- | | | | | |
| | | | | | ketamine | | | | | |
| | | | | | (30 mg/kg) | | | | | |
| | | | | | Saline vs. | — | — | 0.1313 | — | |
| | | | | | FENM | | | | | |
| | | | | | (10 mg/kg) | | | | | |
| | | | | | Saline vs. | — | — | 0.0150 | * | |
| | | | | | FENM | | | | | |
| | | | | | (20 mg/kg) | | | | | |
| | | | | | Saline vs. | — | — | 0.0018 | ** | |
| | | | | | FENM | | | | | |
| | | | | | (30 mg/kg) | | | | | |
| | | | Freezing | Fisher's | Saline vs. | — | — | 0.0101 | * | |
| | | | Min 1 (%) | LSD | (R,S)- | | | | | |
| | | | | | ketamine | | | | | |
| | | | | | (30 mg/kg) | | | | | |
| | | | | | Saline vs. | — | — | 0.4040 | — | |
| | | | | | FENM | | | | | |
| | | | | | (10 mg/kg) | | | | | |
| | | | | | Saline vs. | — | — | 0.2929 | — | |
| | | | | | FENM | | | | | |
| | | | | | (20 mg/kg) | | | | | |
| | | | | | Saline vs. | — | — | 0.0328 | * | |
| | | | | | FENM | | | | | |
| | | | | | (30 mg/kg) | | | | | |
| | | | Freezing | Fisher's | Saline vs. | — | — | 0.0012 | ** | |
| | | | Min 2 (%) | LSD | (R,S)- | | | | | |
| | | | | | ketamine | | | | | |
| | | | | | (30 mg/kg) | | | | | |
| | | | | | Saline vs. | — | — | 0.0961 | — | |
| | | | | | FENM | | | | | |
| | | | | | (10 mg/kg) | | | | | |
| | | | | | Saline vs. | — | — | 0.0225 | * | |
| | | | | | FENM | | | | | |
| | | | | | (20 mg/kg) | | | | | |
| | | | | | Saline vs. | — | — | 0.0080 | ** | |
| | | | | | FENM | | | | | |
| | | | | | (30 mg/kg) | | | | | |
| | | | Freezing | Fisher's | Saline vs. | — | — | 0.0320 | * | |
| | | | Min 3 (%) | LSD | (R,S)- | | | | | |
| | | | | | ketamine | | | | | |
| | | | | | (30 mg/kg) | | | | | |
| | | | | | Saline vs. | — | — | 0.5544 | — | |
| | | | | | FENM | | | | | |
| | | | | | (10 mg/kg) | | | | | |
| | | | | | Saline vs. | — | — | 0.0834 | — | |
| | | | | | FENM | | | | | |
| | | | | | (20 mg/kg) | | | | | |
| | | | | | Saline vs. | — | — | 0.0038 | ** | |
| | | | | | FENM | | | | | |
| | | | | | (30 mg/kg) | | | | | |
| | | | Freezing | Fisher's | Saline vs. | — | — | 0.0592 | — | |
| | | | Min 4 (%) | LSD | (R,S)- | | | | | |
| | | | | | ketamine | | | | | |
| | | | | | (30 mg/kg) | | | | | |
| | | | | | Saline vs. | — | — | 0.0419 | * | |
| | | | | | FENM | | | | | |
| | | | | | (10 mg/kg) | | | | | |
| | | | | | Saline vs. | — | — | 0.0743 | — | |
| | | | | | FENM | | | | | |
| | | | | | (20 mg/kg) | | | | | |
| | | | | | Saline vs. | — | — | 0.0005 | *** | |
| | | | | | FENM | | | | | |
| | | | | | (30 mg/kg) | | | | | |
| | | | Freezing | Fisher's | Saline vs. | — | — | 0.4016 | — | |
| | | | Min 5 (%) | LSD | (R,S)- | | | | | |
| | | | | | ketamine | | | | | |
| | | | | | (30 mg/kg) | | | | | |
| | | | | | Saline vs. | — | — | 0.3855 | — | |
| | | | | | FENM | | | | | |
| | | | | | (10 mg/kg) | | | | | |
| | | | | | Saline vs. | — | — | 0.0027 | ** | |
| | | | | | FENM | | | | | |
| | | | | | (20 mg/kg) | | | | | |
| | | | | | Saline vs. | — | — | 0.0807 | — | |
| | | | | | FENM | | | | | |
| | | | | | (30 mg/kg) | | | | | |
| | | | Freezing | ANOVA | Drug | 3.557 | 4.40 | 0.0142 | * | 1D |
| | | | (%) | Fisher's | Saline vs. | — | — | 0.0086 | ** | |
| | | | | LSD | (R,S)- | | | | | |
| | | | | | ketamine | | | | | |
| | | | | | (30 mg/kg) | | | | | |
| | | | | | Saline vs. | — | — | 0.1313 | — | |
| | | | | | FENM | | | | | |
| | | | | | (10 mg/kg) | | | | | |
| | | | | | Saline vs. | — | — | 0.015 | * | |
| | | | | | FENM | | | | | |
| | | | | | (20 mg/kg) | | | | | |
| | | | | | Saline vs. | — | — | 0.0018 | ** | |
| | | | | | FENM | | | | | |
| | | | | | (30 mg/kg) | | | | | |
| | Forced | FST | Immobility | RMANOVA | Drug | 0.850 | 4.40 | 0.5021 | — | 1E |
| | Swim | Day 1 | Time | | Time | 13.650 | 5200 | <0.0001 | *** | |
| | Test Day 1 | | (sec) | | Drug × Time | 1.446 | 20200 | 0.1048 | — | |
| | Forced | FST | Immobility | RMANOVA | Drug | 7.683 | 4.40 | 0.0001 | *** | 1F |
| | Swim | Day 2 | Time | | Time | 3.981 | 5200 | 0.0018 | ** | |
| | Test Day 2 | | (sec) | | Drug × Time | 0.987 | 20200 | 0.4797 | — | |
| | | | | Fisher's | Saline vs. | — | — | 0.0003 | *** | |
| | | | | PLSD | (R,S)- | | | | | |
| | | | | | ketamine | | | | | |
| | | | | | (30 mg/kg) | | | | | |
| | | | | | Saline vs. | — | — | 0.0085 | ** | |
| | | | | | FENM | | | | | |
| | | | | | (10 mg/kg) | | | | | |
| | | | | | Saline vs. | — | — | <0.0001 | *** | |
| | | | | | FENM | | | | | |
| | | | | | (20 mg/kg) | | | | | |
| | | | | | Saline vs. | — | — | 0.0003 | *** | |
| | | | | | FENM | | | | | |
| | | | | | (30 mg/kg) | | | | | |
| | | | Immobility | ANOVA | Drug | 7.364 | 4.40 | 0.0002 | *** | 1G |
| | | | Time | Fisher's | Saline vs. | — | — | 0.0007 | *** | |
| | | | (sec) | PLSD | (R,S)- | | | | | |
| | | | | | ketamine | | | | | |
| | | | | | (30 mg/kg) | | | | | |
| | | | | | Saline vs. | — | — | 0.0307 | * | |
| | | | | | FENM | | | | | |
| | | | | | (10 mg/kg) | | | | | |
| | | | | | Saline vs. | — | — | <0.0001 | *** | |
| | | | | | FENM | | | | | |
| | | | | | (20 mg/kg) | | | | | |
| | | | | | Saline vs. | — | — | 0.0005 | *** | |
| | | | | | FENM | | | | | |
| | | | | | (30 mg/kg) | | | | | |
| | Open | OF | Distance | RMANOVA | Drug | 0.564 | 4.40 | 0.6903 | — | 1H |
| | Field | | Travelled | | Time | 6.837 | 9360 | <0.0001 | *** | |
| | | | (cm) | | Drug × Time | 1.013 | 36360 | 0.4524 | — | |
| | | | | ANOVA | Drug | 0.564 | 4.40 | 0.6903 | — | 1I |
| | | | Time in | ANOVA | Drug | 1.011 | 4.40 | 0.4132 | — | 1J |
| | | | Center | | | | | | | |
| | | | (sec) | | | | | | | |
| | Marble | MB | Marbles | ANOVA | Drug | 1.328 | 4.24 | 0.2882 | — | 1K |
| | Burying | | Buried | | | | | | | |
| | | | (no.) | | | | | | | |
| | Elevated | EPM | Distance | RMANOVA | Drug | 1.914 | 4.40 | 0.1269 | — | 1L |
| | Plus | | Travelled | | Time | 43.090 | 5200 | <0.0001 | **** | |
| | Maze | | (cm) | | Drug × Time | 1.488 | 20200 | 0.0884 | — | |
| | | | Time in | ANOVA | Drug | 1.214 | 4.40 | 0.3201 | — | data not |
| | | | Open | | | | | | | shown |
| | | | Arms (s) | | | | | | | |
| | | | Time in | ANOVA | Drug | 0.649 | 4.40 | 0.6310 | — | data not |
| | | | Closed | | | | | | | shown |
| | | | Arms (s) | | | | | | | |
| | | | Time in | ANOVA | Drug | 0.529 | 4.40 | 0.7148 | — | data not |
| | | | Center (s) | | | | | | | shown |
| | | | Time in | ANOVA | Drug | 0.604 | 4.40 | 0.6620 | — | 1M |
| | | | Center + | | | | | | | |
| | | | Open | | | | | | | |
| | | | Arms (s) | | | | | | | |
| | Novelty | NSF | Fraction | Log-rank | Drug | — | — | 0.1674 | — | 1N |
| | Suppressed | | of mice | (Mantel- | | | | | | |
| | Feeding | | not eating | Cox) test | | | | | | |
| | | | in OF | | | | | | | |
| | | | Latency | ANOVA | Drug | 1.827 | 4.40 | 0.1426 | — | data not |
| | | | to feed in | | | | | | | shown |
| | | | OF (sec) | | | | | | | |
| | | | Fraction | Log-rank | Drug | — | — | 0.3089 | — | data not |
| | | | of mice | (Mantel- | | | | | | shown |
| | | | not eating | Cox) test | | | | | | |
| | | | in HC | | | | | | | |
| | | | Latency | ANOVA | Drug | 0.727 | 4.40 | 0.5788 | — | data not |
| | | | to feed in | | | | | | | shown |
| | | | HC (sec) | | | | | | | |
| | | | Change in | ANOVA | Drug | 0.973 | 4.40 | 0.4332 | — | 1O |
| | | | Weight (g) | | | | | | | |
| | | | Food | ANOVA | Drug | 1.325 | 4.40 | 0.2773 | — | 1P |
| | | | Eaten (g) | | | | | | | |
| FIG. 2, | Contextual | CFC | Freezing | RMANOVA | Drug | 1.735 | 4.25 | 0.1737 | — | 2B |
| FENM 5 | Fear | Training | (%) | | Time | 79.780 | 4100 | <0.0001 | *** | |
| minutes | Conditioning | | | | Drug × Time | 1.263 | 16100 | 0.2361 | — | |
| after | Training | | | | | | | | | |
| stress | Contextual | Contextual | Freezing | RMANOVA | Drug | 1.217 | 4.20 | 0.3347 | — | 2C |
| | Fear | Fear | (%) | | Time | 5.589 | 4.80 | 0.0005 | ** | |
| | Conditioning | Conditioning | | | Drug × Time | 2.122 | 16.80 | 0.0149 | * | |
| | Re-exposure | Re-exposure | | Fisher's | Saline vs. | — | — | 0.4342 | — | |
| | | | | PLSD | (R,S)- | | | | | |
| | | | | | ketamine | | | | | |
| | | | | | (30 mg/kg) | | | | | |
| | | | | | Sal vs. | — | — | 0.0570 | — | |
| | | | | | FENM | | | | | |
| | | | | | (10 mg/kg) | | | | | |
| | | | | | Sal vs. | — | — | 0.1188 | — | |
| | | | | | FENM | | | | | |
| | | | | | (20) | | | | | |
| | | | | | Sal vs. | — | — | 0.2420 | — | |
| | | | | | FENM | | | | | |
| | | | | | (30) | | | | | |
| | | | Freezing | Fisher's | Saline vs. | — | — | 0.6701 | — | |
| | | | Min 1 (%) | PLSD | (R,S)- | | | | | |
| | | | | | ketamine | | | | | |
| | | | | | (30 mg/kg) | | | | | |
| | | | | | Sal vs. | — | — | 0.1019 | — | |
| | | | | | FENM | | | | | |
| | | | | | (10 mg/kg) | | | | | |
| | | | | | Sal vs. | — | — | 0.1072 | — | |
| | | | | | FENM | | | | | |
| | | | | | (20) | | | | | |
| | | | | | Sal vs. | — | — | 0.8647 | — | |
| | | | | | FENM | | | | | |
| | | | | | (30) | | | | | |
| | | | Freezing | Fisher's | Saline vs. | — | — | 0.0587 | — | |
| | | | Min 2 (%) | PLSD | (R,S)- | | | | | |
| | | | | | ketamine | | | | | |
| | | | | | (30 mg/kg) | | | | | |
| | | | | | Sal vs. | — | — | 0.0359 | * | |
| | | | | | FENM | | | | | |
| | | | | | (10 mg/kg) | | | | | |
| | | | | | Sal vs. | — | — | 0.0080 | ** | |
| | | | | | FENM | | | | | |
| | | | | | (20) | | | | | |
| | | | | | Sal vs. | — | — | 0.4119 | — | |
| | | | | | FENM | | | | | |
| | | | | | (30) | | | | | |
| | | | Freezing | Fisher's | Saline vs. | — | — | 0.1358 | — | |
| | | | Min 3 (%) | PLSD | (R,S)- | | | | | |
| | | | | | ketamine | | | | | |
| | | | | | (30 mg/kg) | | | | | |
| | | | | | Sal vs. | — | — | 0.0300 | * | |
| | | | | | FENM | | | | | |
| | | | | | (10 mg/kg) | | | | | |
| | | | | | Sal vs. | — | — | 0.1619 | — | |
| | | | | | FENM | | | | | |
| | | | | | (20) | | | | | |
| | | | | | Sal vs. | — | — | 0.3540 | — | |
| | | | | | FENM | | | | | |
| | | | | | (30) | | | | | |
| | | | Freezing | Fisher's | Saline vs. | — | — | 0.5515 | — | |
| | | | Min 4 (%) | PLSD | (R,S)- | | | | | |
| | | | | | ketamine | | | | | |
| | | | | | (30 mg/kg) | | | | | |
| | | | | | Sal vs. | — | — | 0.1916 | — | |
| | | | | | FENM | | | | | |
| | | | | | (10 mg/kg) | | | | | |
| | | | | | Sal vs. | — | — | 0.5390 | — | |
| | | | | | FENM | | | | | |
| | | | | | (20) | | | | | |
| | | | | | Sal vs. | — | — | 0.0503 | — | |
| | | | | | FENM | | | | | |
| | | | | | (30) | | | | | |
| | | | Freezing | Fisher's | Saline vs. | — | — | 0.1641 | — | |
| | | | Min 5 (%) | PLSD | (R,S)- | | | | | |
| | | | | | ketamine | | | | | |
| | | | | | (30 mg/kg) | | | | | |
| | | | | | Sal vs. | — | — | 0.6909 | — | |
| | | | | | FENM | | | | | |
| | | | | | (10 mg/kg) | | | | | |
| | | | | | Sal vs. | — | — | 0.8801 | — | |
| | | | | | FENM | | | | | |
| | | | | | (20) | | | | | |
| | | | | | Sal vs. | — | — | 0.3078 | — | |
| | | | | | FENM | | | | | |
| | | | | | (30) | | | | | |
| | | | Freezing | ANOVA | Drug | 1.313 | 4.25 | 0.2922 | — | 2D |
| | | | (%) | | | | | | | |
| | Forced | FST | Immobility | RMANOVA | Drug | 1.443 | 4.25 | 0.2494 | — | 2E |
| | Swim | Day 1 | Time (s) | | Time | 20.100 | 5125 | <0.0001 | *** | |
| | Test | | | | Drug × Time | 0.683 | 20125 | 0.8364 | — | |
| | Forced | FST | Immobility | RMANOVA | Drug | 2.403 | 4.25 | 0.0766 | ns | 2F |
| | Swim Test | Day 2 | Time (s) | | Time | 12.520 | 5125 | <0.0001 | **** | |
| | Day 2 | | | | Drug × Time | 3.073 | 20125 | <0.0001 | **** | |
| | | | Immobility | Fisher's | Saline vs. | — | — | 0.7994 | ns | |
| | | | Time Min | PLSD | (R,S)- | | | | | |
| | | | 1 (s) | | ketamine | | | | | |
| | | | | | (30 mg/kg) | | | | | |
| | | | | | Sal vs. | — | — | 0.3105 | ns | |
| | | | | | FENM | | | | | |
| | | | | | (10 mg/kg) | | | | | |
| | | | | | Sal vs. | — | — | 0.0237 | * | |
| | | | | | FENM | | | | | |
| | | | | | (20) | | | | | |
| | | | | | Sal vs. | — | — | 0.9988 | ns | |
| | | | | | FENM | | | | | |
| | | | | | (30) | | | | | |
| | | | Immobility | Fisher's | Saline vs. | — | — | 0.0125 | * | |
| | | | Time Min | PLSD | (R,S)- | | | | | |
| | | | 2 (s) | | ketamine | | | | | |
| | | | | | (30 mg/kg) | | | | | |
| | | | | | Sal vs. | — | — | 0.2275 | ns | |
| | | | | | FENM | | | | | |
| | | | | | (10 mg/kg) | | | | | |
| | | | | | Sal vs. | — | — | 0.0034 | ** | |
| | | | | | FENM | | | | | |
| | | | | | (20) | | | | | |
| | | | | | Sal vs. | — | — | <0.0001 | **** | |
| | | | | | FENM | | | | | |
| | | | | | (30) | | | | | |
| | | | Immobility | Fisher's | Saline vs. | — | — | 0.8598 | ns | |
| | | | Time Min | PLSD | ketamine | | | | | |
| | | | 3 (s) | | (30 mg/kg) | | | | | |
| | | | | | Sal vs. | — | — | 0.0949 | ns | |
| | | | | | FENM | | | | | |
| | | | | | (10 mg/kg) | | | | | |
| | | | | | Sal vs. | — | — | 0.0763 | ns | |
| | | | | | FENM | | | | | |
| | | | | | (20) | | | | | |
| | | | | | Sal vs. | — | — | 0.2804 | ns | |
| | | | | | FENM | | | | | |
| | | | | | (30) | | | | | |
| | | | Immobility | Fisher's | Saline vs. | — | — | 0.2874 | ns | |
| | | | Time Min | PLSD | (R,S)- | | | | | |
| | | | 4 (s) | | ketamine | | | | | |
| | | | | | (30 mg/kg) | | | | | |
| | | | | | Sal vs. | — | — | 0.1329 | ns | |
| | | | | | FENM | | | | | |
| | | | | | (10 mg/kg) | | | | | |
| | | | | | Sal vs. | — | — | 0.1586 | ns | |
| | | | | | FENM | | | | | |
| | | | | | (20) | | | | | |
| | | | | | Sal vs. | — | — | 0.4890 | ns | |
| | | | | | FENM | | | | | |
| | | | | | (30) | | | | | |
| | | | Immobility | Fisher's | Saline vs. | — | — | 0.6526 | ns | |
| | | | Time Min | PLSD | (R,S)- | | | | | |
| | | | 5 (s) | | ketamine | | | | | |
| | | | | | (30 mg/kg) | | | | | |
| | | | | | Sal vs. | — | — | 0.4763 | ns | |
| | | | | | FENM | | | | | |
| | | | | | (10 mg/kg) | | | | | |
| | | | | | Sal vs. | — | — | 0.0575 | ns | |
| | | | | | FENM | | | | | |
| | | | | | (20) | | | | | |
| | | | | | Sal vs. | — | — | 0.8673 | ns | |
| | | | | | FENM | | | | | |
| | | | | | (30) | | | | | |
| | | | Immobility | Fisher's | Saline vs. | — | — | 0.1586 | ns | |
| | | | Time Min | PLSD | (R,S)- | | | | | |
| | | | 6 (s) | | ketamine | | | | | |
| | | | | | (30 mg/kg) | | | | | |
| | | | | | Sal vs. | — | — | 0.4453 | ns | |
| | | | | | FENM | | | | | |
| | | | | | (10 mg/kg) | | | | | |
| | | | | | Sal vs. | — | — | 0.2137 | ns | |
| | | | | | FENM | | | | | |
| | | | | | (20) | | | | | |
| | | | | | Sal vs. | — | — | 0.6591 | ns | |
| | | | | | FENM | | | | | |
| | | | | | (30) | | | | | |
| | | | Immobility | ANOVA | Drug | 3.148 | 4.25 | 0.0317 | * | 2G |
| | | | Time (s) | Fisher's | Saline vs. | — | — | 0.0466 | * | |
| | | | | PLSD | (R,S)- | | | | | |
| | | | | | ketamine | | | | | |
| | | | | | (30 mg/kg) | | | | | |
| | | | | | Sal vs. | — | — | 0.0204 | * | |
| | | | | | FENM | | | | | |
| | | | | | (10 mg/kg) | | | | | |
| | | | | | Sal vs. | — | — | 0.0024 | ** | |
| | | | | | FENM | | | | | |
| | | | | | (20) | | | | | |
| | | | | | Sal vs. | — | — | 0.0210 | * | |
| | | | | | FENM | | | | | |
| | | | | | (30) | | | | | |
| | Open | OF | Distance | RMANOVA | Drug | 0.503 | 4.25 | 0.7341 | ns | 2H |
| | Field | | Travelled | | Time | 19.300 | 9225 | <0.0001 | **** | |
| | | | (cm) | | Drug × Time | 0.733 | 36225 | 0.8676 | ns | |
| | | | | ANOVA | Drug | 0.503 | 4.25 | 0.7341 | ns | 2I |
| | | | Time in | ANOVA | Drug | 2.207 | 4.25 | 0.0973 | ns | 2J |
| | | | Center (s) | | | | | | | |
| | Marble | MB | Marbles | ANOVA | Drug | 7.590 | 4.25 | 0.0004 | *** | 2K |
| | Burying | | Buried | Fisher's | Saline vs. | — | — | 0.7292 | ns | |
| | | | (no.) | PLSD | (R,S)- | | | | | |
| | | | | | ketamine | | | | | |
| | | | | | (30 mg/kg) | | | | | |
| | | | | | Sal vs. | — | — | 0.0737 | ns | |
| | | | | | FENM | | | | | |
| | | | | | (10 mg/kg) | | | | | |
| | | | | | Sal vs. | — | — | 0.0002 | *** | |
| | | | | | FENM | | | | | |
| | | | | | (20) | | | | | |
| | | | | | Sal vs. | — | — | 0.0097 | ** | |
| | | | | | FENM | | | | | |
| | | | | | (30) | | | | | |
| | Elevated | EMP | Distance | RMANOVA | Drug | 0.662 | 4.25 | 0.6239 | ns | 2L |
| | Plus | | Travelled | | Time | 18.450 | 5125 | <0.0001 | **** | |
| | Maze | | (cm) | | Drug × Time | 0.751 | 20125 | 0.7661 | ns | |
| | | | Time in | ANOVA | Drug | 1.083 | 4.25 | 0.3863 | — | data not |
| | | | Open | | | | | | | shown |
| | | | Arms (s) | | | | | | | |
| | | | Time in | ANOVA | Drug | 1.473 | 4.25 | 0.2401 | — | data not |
| | | | Closed | | | | | | | shown |
| | | | Arms (s) | | | | | | | |
| | | | Time in | ANOVA | Drug | 1.342 | 4.25 | 0.2822 | — | data not |
| | | | Center (s) | | | | | | | shown |
| | | | Time in | ANOVA | Drug | 1.473 | 4.25 | 0.2401 | ns | 2M |
| | | | Center + | | | | | | | |
| | | | Open | | | | | | | |
| | | | Arms (s) | | | | | | | |
| | Novelty- | NSF | Fraction | Log-rank | Drug | — | — | 0.4815 | ns | 2N |
| | Suppressed | | of mice | (Mantel- | | | | | | |
| | Feeding | | not eating | Cox) test | | | | | | |
| | | | Latency | ANOVA | Drug | 0.892 | 4.25 | 0.4833 | ns | data not |
| | | | to feed | | | | | | | shown |
| | | | (sec) | | | | | | | |
| | | | Fraction | Log-rank | Drug | — | — | 0.0865 | — | data not |
| | | | of mice | (Mantel- | | | | | | shown |
| | | | not eating | Cox) test | | | | | | |
| | | | in HC | | | | | | | |
| | | | Latency | ANOVA | Drug | 1.833 | 4.25 | 0.1539 | — | data not |
| | | | to feed in | | | | | | | shown |
| | | | HC (sec) | | | | | | | |
| | | | Change in | ANOVA | Drug | 2.176 | 4.25 | 0.1010 | ns | 2O |
| | | | Weight (g) | | | | | | | |
| | | | Food | ANOVA | Drug | 0.125 | 4.25 | 0.9723 | ns | 2P |
| | | | Eaten (g) | | | | | | | |
| FIG. 3, | Contextual | CFC | Freezing | RMANOVA | Drug | 1.097 | 2.15 | 0.3591 | — | 3B |
| FENM 5 | Fear | Training | (%) | | Time | 39.850 | 4.60 | <0.0001 | *** | |
| minutes | Conditioning | | | | Drug × Time | 0.809 | 8.60 | 0.5971 | — | |
| after | Training | | | | | | | | | |
| stress | | | | ANOVA | Drug | 1.097 | 2.15 | 0.3591 | — | 3C |
| | Contextual | CFC | Freezing | RMANOVA | Drug | 1.952 | 2.15 | 0.1764 | — | 3D |
| | Fear | Re-exposure 1 | (%) | | Time | 41.510 | 4.60 | <0.0001 | *** | |
| | Conditioning | | | | Drug × Time | 0.683 | 8.60 | 0.7048 | — | |
| | Re-exposure 1 | | | ANOVA | Drug | 1.952 | 2.15 | 0.1764 | — | 3E |
| | Contextual | CFC | Freezing | RMANOVA | Drug | 1.296 | 2.15 | 0.3025 | — | 3F |
| | Fear | Re-exposure 2 | (%) | | Time | 10.510 | 4.60 | <0.0001 | *** | |
| | Conditioning | | | | Drug × Time | 1.307 | 8.60 | 0.2575 | — | |
| | Re-exposure 2 | | | ANOVA | Drug | 1.296 | 2.15 | 0.3025 | — | 3G |
| | Forced | FST | Immobility | RMANOVA | Drug | 0.070 | 2.15 | 0.9332 | — | 3H |
| | Swim | Day 1 | Time (s) | | Time | 13.100 | 5.75 | <0.0001 | *** | |
| | Test | | | | Drug × Time | 0.618 | 10.75 | 0.7942 | — | |
| | Day 1 | | | ANOVA | Drug | 0.024 | 2.15 | 0.9767 | — | 3I |
| | Forced | FST | Immobility | RMANOVA | Drug | 7.447 | 2.15 | 0.0057 | ** | 3J |
| | Swim | Day 2 | Time (s) | | Time | 10.910 | 5.75 | <0.0001 | *** | |
| | Test | | | | Drug × Time | 1.544 | 10.75 | 0.1407 | — | |
| | Day 2 | | | Fisher's | Saline vs. | — | — | 0.8125 | — | |
| | | | | PLSD | (R,S)- | | | | | |
| | | | | | ketamine | | | | | |
| | | | | | (30 mg/kg) | | | | | |
| | | | | | Saline vs. | — | — | 0.0234 | * | |
| | | | | | FENM | | | | | |
| | | | | | (20 mg/kg) | | | | | |
| | | | | | (R,S)- | — | — | 0.0069 | ** | |
| | | | | | ketamine | | | | | |
| | | | | | (30 mg/kg) | | | | | |
| | | | | | vs. FENM | | | | | |
| | | | | | (20 mg/kg) | | | | | |
| | | | Immobility | ANOVA | Drug | 10.700 | 2.15 | 0.0013 | ** | 3K |
| | | | Time (s) | Fisher's | Saline vs. | — | — | 0.6082 | — | |
| | | | | PLSD | (R,S)- | | | | | |
| | | | | | ketamine | | | | | |
| | | | | | (30 mg/kg) | | | | | |
| | | | | | Saline vs. | — | — | 0.0097 | ** | |
| | | | | | FENM | | | | | |
| | | | | | (20 mg/kg) | | | | | |
| | | | | | (R,S)- | — | — | 0.0014 | ** | |
| | | | | | ketamine | | | | | |
| | | | | | (30 mg/kg) | | | | | |
| | | | | | vs. FENM | | | | | |
| | | | | | (20 mg/kg) | | | | | |
| | Open | OF | Distance | RMANOVA | Drug | 0.036 | 2.15 | 0.9650 | — | 3L |
| | Field | | Travelled | | Time | 9.389 | 9135 | <0.0001 | *** | |
| | | | (cm) | | Drug × Time | 3.089 | 18135 | <0.0001 | *** | |
| | | | | Fisher's | Saline vs. | — | — | 0.9319 | — | |
| | | | | PLSD | (R,S)- | | | | | |
| | | | | | ketamine | | | | | |
| | | | | | (30 mg/kg) | | | | | |
| | | | | | Saline vs. | — | — | 0.7967 | — | |
| | | | | | FENM | | | | | |
| | | | | | (20 mg/kg) | | | | | |
| | | | | | (R,S)- | — | — | 0.8632 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.0589 | — | |
| | | | Travelled | PLSD | (R,S)- | | | | | |
| | | | Min 1 | | ketamine | | | | | |
| | | | (cm) | | (30 mg/kg) | | | | | |
| | | | | | Saline vs. | — | — | 0.0158 | * | |
| | | | | | FENM | | | | | |
| | | | | | (20 mg/kg) | | | | | |
| | | | | | (R,S)- | — | — | 0.5911 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.0098 | ** | |
| | | | Travelled | PLSD | (R,S)- | | | | | |
| | | | Min 2 | | ketamine | | | | | |
| | | | (cm) | | (30 mg/kg) | | | | | |
| | | | | | Saline vs. | — | — | 0.0068 | ** | |
| | | | | | FENM | | | | | |
| | | | | | (20 mg/kg) | | | | | |
| | | | | | (R,S)- | — | — | 0.8997 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.1586 | — | |
| | | | Travelled | PLSD | (R,S)- | | | | | |
| | | | Min 3 | | ketamine | | | | | |
| | | | (cm) | | (30 mg/kg) | | | | | |
| | | | | | Saline vs. | — | — | 0.4093 | — | |
| | | | | | FENM | | | | | |
| | | | | | (20 mg/kg) | | | | | |
| | | | | | (R,S)- | — | — | 0.5565 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.6504 | — | |
| | | | Travelled | PLSD | (R,S)- | | | | | |
| | | | Min 4 | | ketamine | | | | | |
| | | | (cm) | | (30 mg/kg) | | | | | |
| | | | | | Saline vs. | — | — | 0.4054 | — | |
| | | | | | FENM | | | | | |
| | | | | | (20 mg/kg) | | | | | |
| | | | | | (R,S)- | — | — | 0.7043 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.3454 | — | |
| | | | Travelled | PLSD | (R,S)- | | | | | |
| | | | Min 5 | | ketamine | | | | | |
| | | | (cm) | | (30 mg/kg) | | | | | |
| | | | | | Saline vs. | — | — | 0.1372 | — | |
| | | | | | FENM | | | | | |
| | | | | | (20 mg/kg) | | | | | |
| | | | | | (R,S)- | — | — | 0.5847 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.7410 | — | |
| | | | Travelled | PLSD | (R,S)- | | | | | |
| | | | Min 6 | | ketamine | | | | | |
| | | | (cm) | | (30 mg/kg) | | | | | |
| | | | | | Saline vs. | — | — | 0.5067 | — | |
| | | | | | FENM | | | | | |
| | | | | | (20 mg/kg) | | | | | |
| | | | | | (R,S)- | — | — | 0.7385 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.1344 | — | |
| | | | Travelled | PLSD | (R,S)- | | | | | |
| | | | Min 7 | | ketamine | | | | | |
| | | | (cm) | | (30 mg/kg) | | | | | |
| | | | | | Saline vs. | — | — | 0.4644 | — | |
| | | | | | FENM | | | | | |
| | | | | | (20 mg/kg) | | | | | |
| | | | | | (R,S)- | — | — | 0.4416 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.1467 | — | |
| | | | Travelled | PLSD | (R,S)- | | | | | |
| | | | Min 8 | | ketamine | | | | | |
| | | | (cm) | | (30 mg/kg) | | | | | |
| | | | | | Saline vs. | — | — | 0.0809 | — | |
| | | | | | FENM | | | | | |
| | | | | | (20 mg/kg) | | | | | |
| | | | | | (R,S)- | — | — | 0.7658 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.0061 | ** | |
| | | | Travelled | PLSD | (R,S)- | | | | | |
| | | | Min 9 | | ketamine | | | | | |
| | | | (cm) | | (30 mg/kg) | | | | | |
| | | | | | Saline vs. | — | — | 0.0040 | ** | |
| | | | | | FENM | | | | | |
| | | | | | (20 mg/kg) | | | | | |
| | | | | | (R,S)- | — | — | 0.8899 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.4667 | — | |
| | | | Travelled | PLSD | (R,S)- | | | | | |
| | | | Min 10 | | ketamine | | | | | |
| | | | (cm) | | (30 mg/kg) | | | | | |
| | | | | | Saline vs. | — | — | 0.5094 | — | |
| | | | | | FENM | | | | | |
| | | | | | (20 mg/kg) | | | | | |
| | | | | | (R,S)- | — | — | 0.9456 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Time in | ANOVA | Drug | 0.156 | 2.15 | 0.8567 | — | 3M |
| | | | Center (s) | | | | | | | |
| | Marble | MB | Marbles | ANOVA | Drug | 0.325 | 2.15 | 0.7278 | — | 3N |
| | Burying | | Buried | | | | | | | |
| | | | (no.) | | | | | | | |
| | Elevated | EPM | Time in | ANOVA | Drug | 0.564 | 2.15 | 0.5806 | — | 3O |
| | Plus | | Open | | | | | | | |
| | Maze | | Arms + | | | | | | | |
| | | | Center (s) | | | | | | | |
| | | | Time in | ANOVA | Drug | 2.404 | 2.15 | 0.1242 | — | data not |
| | | | Open | | | | | | | shown |
| | | | Arms (s) | | | | | | | |
| | | | Time in | ANOVA | Drug | 0.825 | 2.15 | 0.4573 | — | data not |
| | | | Closed | | | | | | | shown |
| | | | Arms (s) | | | | | | | |
| | | | Time in | ANOVA | Drug | 0.296 | 2.15 | 0.7848 | — | data not |
| | | | Center (s) | | | | | | | shown |
| | Novelty- | NSF | Fraction | Log-rank | Drug | — | — | 0.1501 | — | 3P |
| | Suppressed | | of mice | (Mantel- | | | | | | |
| | Feeding | | not eating | Cox) test | | | | | | |
| | | | in OF | | | | | | | |
| | | | Latency | ANOVA | Drug | 1.131 | 2.15 | 0.3488 | — | |
| | | | to feed in | | | | | | | |
| | | | OF (sec) | | | | | | | |
| | | | Fraction | Log-rank | Drug | — | — | 0.6325 | — | 3Q |
| | | | of mice | (Mantel- | | | | | | |
| | | | not eating | Cox) test | | | | | | |
| | | | in HC | | | | | | | |
| | | | Latency | ANOVA | Drug | 0.467 | 2.15 | 0.6360 | — | |
| | | | to feed in | | | | | | | |
| | | | HC (sec) | | | | | | | |
| | | | Change in | ANOVA | Drug | 1.954 | 2.15 | 0.1762 | — | 3R |
| | | | Weight (g) | | | | | | | |
| | | | Food | ANOVA | Drug | 0.396 | 2.15 | 0.6798 | — | 3S |
| | | | Eaten (g) | | | | | | | |
| 5 min | Contextual | CFC | Freezing | RMANOVA | Drug | 0.114 | 2.25 | 0.8930 | — | 4B |
| after | Fear | Training | (%) | | Time | 57.920 | 4100 | <0.0001 | *** | |
| re-exposure 1 | Conditioning | | | | Drug × Time | 0.207 | 8100 | 0.9890 | — | |
| | Training | | | ANOVA | Drug | 0.114 | 2.25 | 0.8930 | — | 4C |
| | Contextual | CFC | Freezing | RMANOVA | Drug | 0.106 | 2.29 | 0.9002 | — | 4D |
| | Fear | Re-exposu | (%) | | Time | 18.970 | 4116 | <0.0001 | **** | |
| | | | | | Drug × Time | 0.746 | 8116 | 0.6512 | — | |
| | | | | ANOVA | Drug | 0.106 | 2.29 | 0.9002 | — | 4E |
| | Context | CFC | Freezing | RMANOVA | Drug | 3.680 | 2.29 | 0.0376 | * | 4F |
| | Fear | Re-exposure 2 | (%) | | Time | 32.490 | 4116 | <0.0001 | **** | |
| | Conditioning | | | | Drug × Time | 0.533 | 8116 | 0.8293 | — | |
| | Re-exposure 2 | | | Fisher's | Saline vs. | — | — | 0.9122 | — | |
| | | | | LSD | ketamine | | | | | |
| | | | | | (30 mg/kg) | | | | | |
| | | | | | Saline vs. | — | — | 0.0281 | * | |
| | | | | | FENM | | | | | |
| | | | | | (20 mg/kg) | | | | | |
| | | | | | (R,S)- | — | — | 0.0248 | * | |
| | | | | | ketamine | | | | | |
| | | | | | (30 mg/kg) | | | | | |
| | | | | | vs. FENM | | | | | |
| | | | | | (20 mg/kg) | | | | | |
| | | | | ANOVA | Drug | 3.680 | 2.29 | 0.0376 | * | 4G |
| | | | | Fisher's | Saline vs. | — | — | 0.9122 | — | |
| | | | | LSD | (R,S)- | | | | | |
| | | | | | ketamine | | | | | |
| | | | | | (30 mg/kg) | | | | | |
| | | | | | Saline vs. | — | — | 0.0281 | * | |
| | | | | | FENM | | | | | |
| | | | | | (20 mg/kg) | | | | | |
| | | | | | (R,S)- | — | — | 0.0248 | * | |
| | | | | | ketamine | | | | | |
| | | | | | (30 mg/kg) | | | | | |
| | | | | | vs. FENM | | | | | |
| | | | | | (20 mg/kg) | | | | | |
| | Forced | FST | Immobility | RMANOVA | Drug | 0.028 | 2.29 | 0.9725 | — | 4H |
| | Swim | Day 1 | Time | | Time | 11.460 | 5145 | <0.0001 | *** | |
| | Test | | (sec) | | Drug × Time | 1.322 | 10145 | 0.2239 | — | |
| | Day 1 | | Immobility | ANOVA | Drug | 0.163 | 2.29 | 0.8504 | — | 4I |
| | | | Time | | | | | | | |
| | | | (sec) | | | | | | | |
| | Forced | FST | Immobility | RMANOVA | Drug | 1.062 | 2.29 | 0.3587 | — | 4J |
| | Swim | Day 2 | Time | | Time | 18.790 | 5145 | <0.0001 | *** | |
| | Test | | (sec) | | Drug × Time | 1.855 | 10145 | 0.0562 | — | |
| | Day 2 | | Immobility | ANOVA | Drug | 2.306 | 2.29 | 0.1177 | — | 4K |
| | | | Time | | | | | | | |
| | | | (sec) | | | | | | | |
| | Open | OF | Distance | RMANOVA | Drug | 0.998 | 2.29 | 0.3808 | — | 4L |
| | Field | | Travelled | | Time | 11.150 | 9261 | <0.0001 | *** | |
| | | | (cm) | | Drug × Time | 0.769 | 18261 | 0.7361 | — | |
| | | | Time in | ANOVA | Drug | 0.998 | 2.29 | 0.3808 | — | 4M |
| | | | Center | | | | | | | |
| | | | (sec) | | | | | | | |
| | Marble | MB | Marbles | ANOVA | Drug | 2.691 | 2.29 | 0.0847 | — | 4N |
| | Burying | | Buried | | | | | | | |
| | | | (no.) | | | | | | | |
| | Elevated | EPM | Time in | ANOVA | Drug | 0.007 | 2.29 | 0.9934 | — | 4O |
| | Plus | | Center + | | | | | | | |
| | Maze | | Open | | | | | | | |
| | | | Arms (s) | | | | | | | |
| | | | Time in | ANOVA | Drug | 1.294 | 2.29 | 0.2896 | — | data not |
| | | | Open | | | | | | | shown |
| | | | Arms | | | | | | | |
| | | | (sec) | | | | | | | |
| | | | Time in | ANOVA | Drug | 0.034 | 2.29 | 0.9666 | — | data not |
| | | | Closed | | | | | | | shown |
| | | | Arms | | | | | | | |
| | | | (sec) | | | | | | | |
| | | | Time in | ANOVA | Drug | 0.487 | 2.29 | 0.6193 | — | data not |
| | | | Center | | | | | | | shown |
| | | | (sec) | | | | | | | |
| | Novelty- | NSF | Fraction | Log-rank | Drug | — | — | 0.8020 | — | 4P |
| | Suppressed | | of mice | (Mantel- | | | | | | |
| | Feeding | | not eating | Cox) test | | | | | | |
| | | | in OF | | | | | | | |
| | | | Latency | ANOVA | Drug | 0.136 | 2.29 | 0.8735 | — | |
| | | | to feed in | | | | | | | |
| | | | OF (sec) | | | | | | | |
| | | | Fraction | Log-rank | Drug | — | — | 0.2283 | — | 4Q |
| | | | of mice | (Mantel- | | | | | | |
| | | | not eating | Cox) test | | | | | | |
| | | | in HC | | | | | | | |
| | | | Latency | ANOVA | Drug | 0.914 | 2.29 | 0.4123 | — | |
| | | | to feed in | | | | | | | |
| | | | HC (sec) | | | | | | | |
| | | | Change in | ANOVA | Drug | 1.207 | 2.29 | 0.3138 | — | 4R |
| | | | Weight (g) | | | | | | | |
| | | | Food | ANOVA | Drug | 0.933 | 2.29 | 0.4048 | — | 4S |
| | | | Eaten (g) | | | | | | | |
| Baseline, | Forced | FST | Immobility | RMANOVA | Drug | 0.246 | 1.8 | 0.6334 | — | 6B |
| [symbol] | Swim | Day 1 | Time | | Time | 8.309 | 5.40 | <0.0001 | *** | |
| | Test | | (sec) | | Drug × Time | 0.339 | 5.40 | 0.8861 | — | |
| | | | | t-test | Saline vs. | — | — | 0.7086 | — | 6C |
| | | | | | FENM | | | | | |
| | | | | | (20 mg/kg) | | | | | |
| | Forced | FST | Immobility | RMANOVA | Drug | 0.030 | 1.8 | 0.8657 | — | 6D |
| | Swim | Day 2 | Time | | Time | 2.593 | 5.40 | 0.0401 | * | |
| | Test | | (sec) | | Drug × Time | 2.949 | 5.40 | 0.0233 | * | |
| | Day 2 | | Immobility | Fisher's | Saline vs. | — | — | 0.3330 | — | |
| | | | Time Min | LSD | FENM | | | | | |
| | | | 1 (sec) | | (20 mg/kg) | | | | | |
| | | | Immobility | Fisher's | Saline vs. | — | — | 0.4156 | — | |
| | | | Time Min | LSD | FENM | | | | | |
| | | | 2 (sec) | | (20 mg/kg) | | | | | |
| | | | Immobility | Fisher's | Saline vs. | — | — | 0.0346 | * | |
| | | | Time Min | LSD | FENM | | | | | |
| | | | 3 (sec) | | (20 mg/kg) | | | | | |
| | | | Immobility | Fisher's | Saline vs. | — | — | 0.2905 | — | |
| | | | Time Min | LSD | FENM | | | | | |
| | | | 4 (sec) | | (20 mg/kg) | | | | | |
| | | | Immobility | Fisher's | Saline vs. | — | — | 0.6448 | — | |
| | | | Time Min | LSD | FENM | | | | | |
| | | | 5 (sec) | | (20 mg/kg) | | | | | |
| | | | Immobility | Fisher's | Saline vs. | — | — | 0.5256 | — | |
| | | | Time Min | LSD | FENM | | | | | |
| | | | 6 (sec) | | (20 mg/kg) | | | | | |
| | | | Immobility | t-test | Saline vs. | — | — | 0.7847 | — | 6E |
| | | | Time | | FENM | | | | | |
| | | | (sec) | | (20 mg/kg) | | | | | |
| Baseline, | Open | OF | Distance | RMANOVA | Drug | 1.885 | 1.18 | 0.1866 | — | 7B |
| anxiety | Field | | Travelled | | Time | 5.317 | 9162 | <0.0001 | *** | |
| | | | (cm) | | Drug × Time | 0.541 | 9162 | 0.8429 | — | |
| | | | Distance | t-test | Saline vs. | — | — | 0.1878 | — | 7C |
| | | | Travelled | | FENM | | | | | |
| | | | (cm) | | (20 mg/kg) | | | | | |
| | | | Time in | t-test | Saline vs. | — | — | 0.1241 | — | 7D |
| | | | Center | | FENM | | | | | |
| | | | (sec) | | (20 mg/kg) | | | | | |
| | | | Time in | t-test | Saline vs. | — | — | 0.1225 | — | 7E |
| | | | Periphery | | FENM | | | | | |
| | | | (sec) | | (20 mg/kg) | | | | | |
| | Marble | MB | Marbles | t-test | Saline vs. | — | — | 0.4657 | — | 7F |
| | Burying | | Buried | | FENM | | | | | |
| | | | (no.) | | (20 mg/kg) | | | | | |
| | Elevated | EPM | Time in | t-test | Saline vs. | — | — | 0.5729 | — | 7G |
| | Plus | | Open | | FENM | | | | | |
| | Maze | | Arms | | (20 mg/kg) | | | | | |
| | | | (sec) | | | | | | | |
| | | | Time in | t-test | Saline vs. | — | — | 0.5729 | — | 7H |
| | | | Closed | | FENM | | | | | |
| | | | Arms | | (20 mg/kg) | | | | | |
| | | | (sec) | | | | | | | |
| | | | Time in | t-test | Saline vs. | — | — | 0.3211 | — | 7I |
| | | | Center | | FENM | | | | | |
| | | | (sec) | | (20 mg/kg) | | | | | |
| | Novelty- | NSF | Fraction | Log-rank | Saline vs. | — | — | 0.0210 | * | 7J |
| | suppressed | | of mice | (Mantel- | FENM | | | | | |
| | | | not eating | Cox) test | (20 mg/kg) | | | | | |
| | | | in OF | | | | | | | |
| | | | Fraction | Log-rank | Saline vs. | — | — | 0.7953 | — | 7K |
| | | | of mice | (Mantel- | FENM | | | | | |
| | | | not eating | Cox) test | (20 mg/kg) | | | | | |
| | | | in HC | | | | | | | |
| | | | Change in | t-test | Saline vs. | — | — | 0.1661 | — | 7L |
| | | | Weight (g) | | FENM | | | | | |
| | | | | | (20 mg/kg) | | | | | |
| | | | Food | t-test | Saline vs. | — | — | 0.0801 | — | 7M |
| | | | Eaten (g) | | FENM | | | | | |
| | | | | | (20 mg/kg) | | | | | |
| 5 min | Contextual | CFC | Freezing | RMANOVA | Drug | 8.624 | 2.15 | 0.0032 | ** | 8B |
| before | Fear | Training | (%) | | Time | 15.430 | 4.60 | <0.0001 | *** | |
| CFC | Conditioning | | | | Drug × Time | 3.146 | 8.60 | 0.0049 | ** | |
| | Training | | | Fisher's | Saline vs. | — | — | 0.0024 | ** | |
| | | | | LSD | (R,S)- | | | | | |
| | | | | | ketamine | | | | | |
| | | | | | (30 mg/kg) | | | | | |
| | | | | | Saline vs. | — | — | 0.9251 | — | |
| | | | | | FENM | | | | | |
| | | | | | (20 mg/kg) | | | | | |
| | | | | | (R,S)- | — | — | 0.0029 | ** | |
| | | | | | ketamine | | | | | |
| | | | | | (30 mg/kg) | | | | | |
| | | | | | vs. FENM | | | | | |
| | | | | | (20 mg/kg) | | | | | |
| | | | Average | ANOVA | Drug | 8.624 | 2.15 | 0.0032 | ** | 8C |
| | | | Freezing | Fisher's | Saline vs. | — | — | 0.0024 | ** | |
| | | | | LSD | (R,S)- | | | | | |
| | | | | | ketamine | | | | | |
| | | | | | (30 mg/kg) | | | | | |
| | | | | | Saline vs. | — | — | 0.9251 | — | |
| | | | | | FENM | | | | | |
| | | | | | (20 mg/kg) | | | | | |
| | | | | | (R,S)- | — | — | 0.0029 | ** | |
| | | | | | ketamine | | | | | |
| | | | | | (30 mg/kg) | | | | | |
| | | | | | vs. FENM | | | | | |
| | | | | | (20 mg/kg) | | | | | |
| | Contextual | CFC | Freezing | RMANOVA | Drug | 67.250 | 2.15 | <0.0001 | *** | 8D |
| | Fear | Re-exposure 1 | (%) | | Time | 8.065 | 4.60 | <0.0001 | *** | |
| | Conditioning | | | | Drug × Time | 4.322 | 8.60 | 0.0004 | *** | |
| | Re-exposure 1 | | | Fisher's | Saline vs. | — | — | <0.0001 | *** | |
| | | | | LSD | (R,S)- | | | | | |
| | | | | | ketamine | | | | | |
| | | | | | (30 mg/kg) | | | | | |
| | | | | | Saline vs. | — | — | 0.0665 | — | |
| | | | | | FENM | | | | | |
| | | | | | (20 mg/kg) | | | | | |
| | | | | | (R,S)- | — | — | <0.0001 | *** | |
| | | | | | ketamine | | | | | |
| | | | | | (30 mg/kg) | | | | | |
| | | | | | vs. FENM | | | | | |
| | | | | | (20 mg/kg) | | | | | |
| | | | Freezing | Fisher's | Saline vs. | — | — | 0.0066 | ** | |
| | | | Min 1 (%) | LSD | (R,S)- | | | | | |
| | | | | | ketamine | | | | | |
| | | | | | (30 mg/kg) | | | | | |
| | | | | | Saline vs. | — | — | 0.0008 | *** | |
| | | | | | FENM | | | | | |
| | | | | | (20 mg/kg) | | | | | |
| | | | | | (R,S)- | — | — | <0.0001 | *** | |
| | | | | | ketamine | | | | | |
| | | | | | (30 mg/kg) | | | | | |
| | | | | | vs. FENM | | | | | |
| | | | | | (20 mg/kg) | | | | | |
| | | | Freezing | Fisher's | Saline vs. | — | — | <0.0001 | *** | |
| | | | Min 2 (%) | LSD | (R,S)- | | | | | |
| | | | | | ketamine | | | | | |
| | | | | | (30 mg/kg) | | | | | |
| | | | | | Saline vs. | — | — | 0.0075 | ** | |
| | | | | | FENM | | | | | |
| | | | | | (20 mg/kg) | | | | | |
| | | | | | (R,S)- | — | — | <0.0001 | *** | |
| | | | | | ketamine | | | | | |
| | | | | | (30 mg/kg) | | | | | |
| | | | | | vs. FENM | | | | | |
| | | | | | (20 mg/kg) | | | | | |
| | | | Freezing | Fisher's | Saline vs. | — | — | <0.0001 | *** | |
| | | | Min 3 (%) | LSD | (R,S)- | | | | | |
| | | | | | ketamine | | | | | |
| | | | | | (30 mg/kg) | | | | | |
| | | | | | Saline vs. | — | — | 0.1384 | — | |
| | | | | | FENM | | | | | |
| | | | | | (20 mg/kg) | | | | | |
| | | | | | (R,S)- | — | — | <0.0001 | *** | |
| | | | | | ketamine | | | | | |
| | | | | | (30 mg/kg) | | | | | |
| | | | | | vs. FENM | | | | | |
| | | | | | (20 mg/kg) | | | | | |
| | | | Freezing | Fisher's | Saline vs. | — | — | <0.0001 | *** | |
| | | | Min 4 (%) | LSD | (R,S)- | | | | | |
| | | | | | ketamine | | | | | |
| | | | | | (30 mg/kg) | | | | | |
| | | | | | Saline vs. | — | — | 0.7601 | — | |
| | | | | | FENM | | | | | |
| | | | | | (20 mg/kg) | | | | | |
| | | | | | (R,S)- | — | — | <0.0001 | *** | |
| | | | | | ketamine | | | | | |
| | | | | | (30 mg/kg) | | | | | |
| | | | | | vs. FENM | | | | | |
| | | | | | (20 mg/kg) | | | | | |
| | | | Freezing | Fisher's | Saline vs. | — | — | <0.0001 | *** | |
| | | | Min 5 (%) | LSD | (R,S)- | | | | | |
| | | | | | ketamine | | | | | |
| | | | | | (30 mg/kg) | | | | | |
| | | | | | Saline vs. | — | — | 0.4751 | — | |
| | | | | | FENM | | | | | |
| | | | | | (20 mg/kg) | | | | | |
| | | | | | (R,S)- | — | — | <0.0001 | *** | |
| | | | | | ketamine | | | | | |
| | | | | | (30 mg/kg) | | | | | |
| | | | | | vs. FENM | | | | | |
| | | | | | (20 mg/kg) | | | | | |
| | | | Freezing | ANOVA | Drug | 67.250 | 2.15 | <0.0001 | *** | 8E |
| | | | (%) | Fisher's | Saline vs. | — | — | <0.0001 | *** | |
| | | | | LSD | (R,S)- | | | | | |
| | | | | | ketamine | | | | | |
| | | | | | (30 mg/kg) | | | | | |
| | | | | | Saline vs. | — | — | 0.0665 | — | |
| | | | | | FENM | | | | | |
| | | | | | (20 mg/kg) | | | | | |
| | | | | | (R,S)- | — | — | <0.0001 | *** | |
| | | | | | ketamine | | | | | |
| | | | | | (30 mg/kg) | | | | | |
| | | | | | vs. FENM | | | | | |
| | | | | | (20 mg/kg) | | | | | |
| | Contextual | CFC | Freezing | RMANOVA | Drug | 35.700 | 2.15 | <0.0001 | *** | 8F |
| | Fear | Re-exposure 2 | (%) | | Time | 14.360 | 4.60 | <0.0001 | *** | |
| | Conditioning | | | | Drug × Time | 3.669 | 8.60 | 0.0015 | ** | |
| | Re-exposure 2 | | | Fisher's | Saline vs. | — | — | <0.0001 | *** | |
| | | | | LSD | (R,S)- | | | | | |
| | | | | | ketamine | | | | | |
| | | | | | (30 mg/kg) | | | | | |
| | | | | | Saline vs. | — | — | 0.0173 | * | |
| | | | | | FENM | | | | | |
| | | | | | (20 mg/kg) | | | | | |
| | | | | | (R,S)- | — | — | <0.0001 | *** | |
| | | | | | ketamine | | | | | |
| | | | | | (30 mg/kg) | | | | | |
| | | | | | vs. FENM | | | | | |
| | | | | | (20 mg/kg) | | | | | |
| | | | Freezing | Fisher's | Saline vs. | — | — | 0.4378 | — | |
| | | | Min 1 (%) | LSD | (R,S)- | | | | | |
| | | | | | ketamine | | | | | |
| | | | | | (30 mg/kg) | | | | | |
| | | | | | Saline vs. | — | — | 0.5432 | — | |
| | | | | | FENM | | | | | |
| | | | | | (20 mg/kg) | | | | | |
| | | | | | (R,S)- | — | — | 0.1684 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Freezing | Fisher's | Saline vs. | — | — | 0.0304 | * | |
| | | | Min 2 (%) | LSD | (R,S)- | | | | | |
| | | | | | ketamine | | | | | |
| | | | | | (30 mg/kg) | | | | | |
| | | | | | Saline vs. | — | — | 0.0173 | * | |
| | | | | | FENM | | | | | |
| | | | | | (20 mg/kg) | | | | | |
| | | | | | (R,S)- | — | — | <0.0001 | *** | |
| | | | | | ketamine | | | | | |
| | | | | | (30 mg/kg) | | | | | |
| | | | | | vs. FENM | | | | | |
| | | | | | (20 mg/kg) | | | | | |
| | | | Freezing | Fisher's | Saline vs. | — | — | 0.0033 | ** | |
| | | | Min 3 (%) | LSD | ketamine | | | | | |
| | | | | | (30 mg/kg) | | | | | |
| | | | | | Saline vs. | — | — | 0.0053 | ** | |
| | | | | | FENM | | | | | |
| | | | | | (20 mg/kg) | | | | | |
| | | | | | (R,S)- | — | — | <0.0001 | *** | |
| | | | | | ketamine | | | | | |
| | | | | | (30 mg/kg) | | | | | |
| | | | | | vs. FENM | | | | | |
| | | | | | (20 mg/kg) | | | | | |
| | | | Freezing | Fisher's | Saline vs. | — | — | <0.0001 | *** | |
| | | | Min 4 (%) | LSD | (R,S)- | | | | | |
| | | | | | ketamine | | | | | |
| | | | | | (30 mg/kg) | | | | | |
| | | | | | Saline vs. | — | — | 0.3068 | — | |
| | | | | | FENM | | | | | |
| | | | | | (20 mg/kg) | | | | | |
| | | | | | (R,S)- | — | — | <0.0001 | *** | |
| | | | | | ketamine | | | | | |
| | | | | | (30 mg/kg) | | | | | |
| | | | | | vs. FENM | | | | | |
| | | | | | (20 mg/kg) | | | | | |
| | | | Freezing | Fisher's | Saline vs. | — | — | <0.0001 | *** | |
| | | | Min 5 (%) | LSD | (R,S)- | | | | | |
| | | | | | ketamine | | | | | |
| | | | | | (30 mg/kg) | | | | | |
| | | | | | Saline vs. | — | — | 0.3729 | — | |
| | | | | | FENM | | | | | |
| | | | | | (20 mg/kg) | | | | | |
| | | | | | (R,S)- | — | — | <0.0001 | *** | |
| | | | | | ketamine | | | | | |
| | | | | | (30 mg/kg) | | | | | |
| | | | | | vs. FENM | | | | | |
| | | | | | (20 mg/kg) | | | | | |
| | | | Freezing | ANOVA | Drug | 35.700 | 1.15 | <0.0001 | *** | 8G |
| | | | (%) | Fisher's | Saline vs. | — | — | <0.0001 | *** | |
| | | | | LSD | (R,S)- | | | | | |
| | | | | | ketamine | | | | | |
| | | | | | (30 mg/kg) | | | | | |
| | | | | | Saline vs. | — | — | 0.0173 | * | |
| | | | | | FENM | | | | | |
| | | | | | (20 mg/kg) | | | | | |
| | | | | | (R,S)- | — | — | <0.0001 | *** | |
| | | | | | ketamine | | | | | |
| | | | | | (30 mg/kg) | | | | | |
| | | | | | vs. FENM | | | | | |
| | | | | | (20 mg/kg) | | | | | |
| | Forced | FSY | Immobility | RMANOVA | Drug | 2.958 | 2.15 | 0.0826 | — | 8H |
| | Swim | Day 1 | Time | | Time | 1.201 | 5.75 | 0.3170 | — | |
| | Test | | (sec) | | Drug × Time | 2.668 | 10.75 | 0.0076 | ** | |
| | Day 1 | | Immobility | Fisher's | Saline vs. | — | — | 0.0026 | ** | |
| | | | Time Min | LSD | (R,S)- | | | | | |
| | | | 1 (sec) | | ketamine | | | | | |
| | | | | | (30 mg/kg) | | | | | |
| | | | | | Saline vs. | — | — | <0.0001 | *** | |
| | | | | | FENM | | | | | |
| | | | | | (20 mg/kg) | | | | | |
| | | | | | (R,S)- | — | — | 0.2548 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Immobility | Fisher's | Saline vs. | — | — | 0.2638 | — | |
| | | | Time Min | LSD | (R,S)- | | | | | |
| | | | 2 (sec) | | ketamine | | | | | |
| | | | | | (30 mg/kg) | | | | | |
| | | | | | Saline vs. | — | — | 0.0752 | — | |
| | | | | | FENM | | | | | |
| | | | | | (20 mg/kg) | | | | | |
| | | | | | (R,S)- | — | — | 0.5009 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Immobility | Fisher's | Saline vs. | — | — | 0.2171 | — | |
| | | | Time Min | LSD | (R,S)- | | | | | |
| | | | 3 (sec) | | ketamine | | | | | |
| | | | | | (30 mg/kg) | | | | | |
| | | | | | Saline vs. | — | — | 0.0422 | * | |
| | | | | | FENM | | | | | |
| | | | | | (20 mg/kg) | | | | | |
| | | | | | (R,S)- | — | — | 0.4155 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Immobility | Fisher's | Saline vs. | — | — | 0.6849 | — | |
| | | | Time Min | LSD | (R,S)- | | | | | |
| | | | 4 (sec) | | ketamine | | | | | |
| | | | | | (30 mg/kg) | | | | | |
| | | | | | Saline vs. | — | — | 0.4348 | — | |
| | | | | | FENM | | | | | |
| | | | | | (20 mg/kg) | | | | | |
| | | | | | (R,S)- | — | — | 0.7067 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Immobility | Fisher's | Saline vs. | — | — | 0.2114 | — | |
| | | | Time Min | LSD | (R,S)- | | | | | |
| | | | 5 (sec) | | ketamine | | | | | |
| | | | | | (30 mg/kg) | | | | | |
| | | | | | Saline vs. | — | — | 0.8223 | — | |
| | | | | | FENM | | | | | |
| | | | | | (20 mg/kg) | | | | | |
| | | | | | (R,S)- | — | — | 0.3041 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Immobility | Fisher's | Saline vs. | — | — | 0.0499 | * | |
| | | | Time Min | LSD | (R,S)- | | | | | |
| | | | 6 (sec) | | ketamine | | | | | |
| | | | | | (30 mg/kg) | | | | | |
| | | | | | Saline vs. | — | — | 0.0677 | — | |
| | | | | | FENM | | | | | |
| | | | | | (20 mg/kg) | | | | | |
| | | | | | (R,S)- | — | — | 0.8903 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Immobility | ANOVA | Drug | 1.646 | 2.15 | 0.2257 | — | 8I |
| | | | Time (s) | | | | | | | |
| | Forced | FST | Immobility | RMANOVA | Drug | 7.857 | 2.15 | 0.0046 | ** | 8J |
| | Swim | Day 2 | Time | | Time | 4.338 | 5.75 | 0.0016 | ** | |
| | Test | | (sec) | | Drug × Time | 1.121 | 10.75 | 0.3583 | — | |
| | Day 2 | | | Fisher's | Saline vs. | — | — | 0.0036 | ** | |
| | | | | LSD | (R,S)- | | | | | |
| | | | | | ketamine | | | | | |
| | | | | | (30 mg/kg) | | | | | |
| | | | | | Saline vs. | — | — | 0.3085 | — | |
| | | | | | FENM | | | | | |
| | | | | | (20 mg/kg) | | | | | |
| | | | | | (R,S)- | — | — | 0.0712 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Immobility | ANOVA | Drug | 7.431 | 2.15 | 0.0057 | ** | 8K |
| | | | Time | Fisher's | Saline vs. | — | — | 0.0046 | ** | |
| | | | (sec) | LSD | (R,S)- | | | | | |
| | | | | | ketamine | | | | | |
| | | | | | (30 mg/kg) | | | | | |
| | | | | | Saline vs. | — | — | 0.3636 | — | |
| | | | | | FENM | | | | | |
| | | | | | (20 mg/kg) | | | | | |
| | | | | | (R,S)- | — | — | 0.0713 | — | |
| | | | | | ketamine | | | | | |
| | | | | | (30 mg/kg) | | | | | |
| | | | | | vs. FENM | | | | | |
| | | | | | (20 mg/kg) | | | | | |
| | Open | OF | Distance | RMANOVA | Drug | 0.040 | 2.15 | 0.9612 | — | 8L |
| | Field | | Travelled | | Time | 3.874 | 9135 | 0.0002 | ** | |
| | | | (cm) | | Drug × Time | 0.935 | 18135 | 0.5379 | — | |
| | | | Time in | ANOVA | Drug | 1.095 | 2.15 | 0.3598 | — | 8M |
| | | | Center | | | | | | | |
| | | | (sec) | | | | | | | |
| | Marble | MB | Marbles | ANOVA | Drug | 1.500 | 2.15 | 0.2548 | — | 8N |
| | Burying | | Buried | | | | | | | |
| | | | (no.) | | | | | | | |
| | Elevated | EPM | Time in | ANOVA | Drug | | | | | 8O |
| | Plus | | Open | | | | | | | |
| | Maze | | Arms + | | | | | | | |
| | | | Center | | | | | | | |
| | | | (sec) | | | | | | | |
| | | | Time in | ANOVA | Drug | 0.282 | 2.15 | 0.7582 | — | data not |
| | | | Open | | | | | | | shown |
| | | | Arms | | | | | | | |
| | | | (sec) | | | | | | | |
| | | | Time in | ANOVA | Drug | 1.900 | 2.15 | 0.1839 | — | data not |
| | | | Closed | | | | | | | shown |
| | | | Arms | | | | | | | |
| | | | (sec) | | | | | | | |
| | | | Time in | ANOVA | Drug | 1.982 | 2.15 | 0.1723 | — | data not |
| | | | Center | | | | | | | shown |
| | | | (sec) | | | | | | | |
| | Novelty | NSF | Fraction | Log-rank | Drug | — | — | 0.0250 | * | 8P |
| | Suppressed | | of mice | (Mantel- | | | | | | |
| | Feeding | | not eating | Cox) test | | | | | | |
| | | | in OF | | | | | | | |
| | | | Latency | ANOVA | Drug | 4.251 | 2.15 | 0.0345 | * | |
| | | | to feed in | Fisher's | Saline vs. | — | — | 0.0575 | ns | |
| | | | OF (sec) | LSD | (R,S)- | | | | | |
| | | | | | ketamine | | | | | |
| | | | | | (30 mg/kg) | | | | | |
| | | | | | Saline vs. | — | — | 0.4585 | ns | |
| | | | | | FENM | | | | | |
| | | | | | (20 mg/kg) | | | | | |
| | | | | | (R,S)- | — | — | 0.0130 | * | |
| | | | | | ketamine | | | | | |
| | | | | | (30 mg/kg) | | | | | |
| | | | | | vs. FENM | | | | | |
| | | | | | (20 mg/kg) | | | | | |
| | | | Fraction | Log-rank | Drug | — | — | 0.5238 | — | 8Q |
| | | | of mice | (Mantel- | | | | | | |
| | | | not eating | Cox) test | | | | | | |
| | | | in HC | | | | | | | |
| | | | Latency | ANOVA | Drug | 0.291 | 2.15 | 0.7514 | — | |
| | | | to feed in | | | | | | | |
| | | | HC (sec) | | | | | | | |
| | | | Change in | ANOVA | Drug | 1.064 | 2.15 | 0.3698 | ns | 8R |
| | | | Weight (g) | | | | | | | |
| | | | Food | ANOVA | Drug | 0.280 | 2.15 | 0.7596 | ns | 8S |
| | | | Eaten (g) | | | | | | | |
| 5 min | Contextual | CFC | Freezing | RMANOVA | Drug | 0.175 | 2.15 | 0.8408 | — | 9B |
| before | Fear | Training | (%) | | Time | 35.910 | 4.60 | <0.0001 | *** | |
| re-exposure 1 | Conditioning | | | | Drug × Time | 0.437 | 8.60 | 0.8942 | — | |
| | Training | | | ANOVA | Drug | 0.175 | 2.15 | 0.8408 | — | 9C |
| | Contextual | CFC | Freezing | RMANOVA | Drug | 0.489 | 2.15 | 0.6228 | — | 9D |
| | Fear | Re-exposure 1 | (%) | | Time | 3.788 | 4.60 | 0.0082 | ** | |
| | Conditioning | | | | Drug × Time | 1.332 | 8.60 | 0.2453 | — | |
| | Re-exposure 1 | | Freezing | ANOVA | Drug | 0.489 | 2.15 | 0.6228 | — | 9E |
| | | | (%) | | | | | | | |
| | Contextual | CFC | Freezing | RMANOVA | Drug | 2.010 | 2.15 | 0.1685 | — | 9F |
| | Fear | Re-exposure 2 | (%) | | Time | 9.659 | 4.60 | <0.0001 | *** | |
| | Conditioning | | | | Drug × Time | 1.350 | 8.60 | 0.2374 | — | |
| | Re-exposure 2 | | Freezing | ANOVA | Drug | 2.010 | 2.15 | 0.1685 | — | 9G |
| | | | (%) | | | | | | | |
| | Forced | FST | Immobility | RMANOVA | Drug | 0.645 | 2.15 | 0.5387 | — | 9H |
| | Swim | Day 1 | Time | | Time | 17.510 | 5.75 | <0.0001 | *** | |
| | Test | | (sec) | | Drug × Time | 10.75 | 1.331 | 0.2298 | — | |
| | Day 1 | | Immobility | ANOVA | Drug | 0.208 | 2.15 | 0.8143 | — | 9I |
| | | | Time | | | | | | | |
| | | | (sec) | | | | | | | |
| | Forced | FST | Immobility | RMANOVA | Drug | 0.627 | 2.15 | 0.5475 | — | 9J |
| | Swim | Day 2 | Time | | Time | 11.830 | 5.75 | <0.0001 | *** | |
| | Test | | (sec) | | Drug × Time | 0.464 | 10.75 | 0.9079 | — | |
| | Day 2 | | Immobility | ANOVA | Drug | 0.472 | 2.15 | 0.6325 | — | 9K |
| | | | Time | | | | | | | |
| | | | (sec) | | | | | | | |
| | Open | OF | Distance | RMANOVA | Drug | 2.498 | 2.15 | 0.1158 | — | 9L |
| | Field | | Travelled | | Time | 14.660 | 9135 | <0.0001 | *** | |
| | | | (cm) | | Drug × Time | 1.758 | 18135 | 0.0368 | * | |
| | | | Distance | Fisher's | Saline vs. | — | — | 0.3306 | — | |
| | | | Travelled | LSD | (R,S)- | | | | | |
| | | | Min 1 | | ketamine | | | | | |
| | | | (cm) | | (30 mg/kg) | | | | | |
| | | | | | Saline vs. | — | — | 0.1253 | — | |
| | | | | | FENM | | | | | |
| | | | | | (20 mg/kg) | | | | | |
| | | | | | (R,S)- | — | — | 0.0129 | * | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.5200 | — | |
| | | | Travelled | LSD | (R,S)- | | | | | |
| | | | Min 2 | | ketamine | | | | | |
| | | | (cm) | | (30 mg/kg) | | | | | |
| | | | | | Saline vs. | — | — | 0.0385 | * | |
| | | | | | FENM | | | | | |
| | | | | | (20 mg/kg) | | | | | |
| | | | | | (R,S)- | — | — | 0.0070 | ** | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.3850 | — | |
| | | | Travelled | LSD | (R,S)- | | | | | |
| | | | Min 3 | | ketamine | | | | | |
| | | | (cm) | | (30 mg/kg) | | | | | |
| | | | | | Saline vs. | — | — | 0.0219 | * | |
| | | | | | FENM | | | | | |
| | | | | | (20 mg/kg) | | | | | |
| | | | | | (R,S)- | — | — | 0.0017 | ** | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.5357 | — | |
| | | | Travelled | LSD | (R,S)- | | | | | |
| | | | Min 4 | | ketamine | | | | | |
| | | | (cm) | | (30 mg/kg) | | | | | |
| | | | | | Saline vs. | — | — | 0.0708 | — | |
| | | | | | FENM | | | | | |
| | | | | | (20 mg/kg) | | | | | |
| | | | | | (R,S)- | — | — | 0.0158 | * | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.9852 | — | |
| | | | Travelled | LSD | (R,S)- | | | | | |
| | | | Min 5 | | ketamine | | | | | |
| | | | (cm) | | (30 mg/kg) | | | | | |
| | | | | | Saline vs. | — | — | 0.0540 | — | |
| | | | | | FENM | | | | | |
| | | | | | (20 mg/kg) | | | | | |
| | | | | | (R,S)- | — | — | 0.0563 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.8156 | — | |
| | | | Travelled | LSD | (R,S)- | | | | | |
| | | | Min 6 | | ketamine | | | | | |
| | | | (cm) | | (30 mg/kg) | | | | | |
| | | | | | Saline vs. | — | — | 0.3753 | — | |
| | | | | | FENM | | | | | |
| | | | | | (20 mg/kg) | | | | | |
| | | | | | (R,S)- | — | — | 0.2633 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.6375 | — | |
| | | | Travelled | LSD | (R,S)- | | | | | |
| | | | Min 7 | | ketamine | | | | | |
| | | | (cm) | | (30 mg/kg) | | | | | |
| | | | | | Saline vs. | — | — | 0.2989 | — | |
| | | | | | FENM | | | | | |
| | | | | | (20 mg/kg) | | | | | |
| | | | | | (R,S)- | — | — | 0.5695 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.2174 | — | |
| | | | Travelled | LSD | (R,S)- | | | | | |
| | | | Min 8 | | ketamine | | | | | |
| | | | (cm) | | (30 mg/kg) | | | | | |
| | | | | | Saline vs. | — | — | 0.4000 | — | |
| | | | | | FENM | | | | | |
| | | | | | (20 mg/kg) | | | | | |
| | | | | | (R,S)- | — | — | 0.6936 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.3052 | — | |
| | | | Travelled | LSD | (R,S)- | | | | | |
| | | | Min 9 | | ketamine | | | | | |
| | | | (cm) | | (30 mg/kg) | | | | | |
| | | | | | Saline vs. | — | — | 0.5188 | — | |
| | | | | | FENM | | | | | |
| | | | | | (20 mg/kg) | | | | | |
| | | | | | (R,S)- | — | — | 0.0959 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Distance | Fisher's | Saline vs. | — | — | 0.8070 | — | |
| | | | Travelled | LSD | (R,S)- | | | | | |
| | | | Min 10 | | ketamine | | | | | |
| | | | (cm) | | (30 mg/kg) | | | | | |
| | | | | | Saline vs. | — | — | 0.6663 | — | |
| | | | | | FENM | | | | | |
| | | | | | (20 mg/kg) | | | | | |
| | | | | | (R,S)- | — | — | 0.4995 | — | |
| | | | | | ketamine | | | | | |
| | | | | | (30 mg/kg) | | | | | |
| | | | | | vs. FENM | | | | | |
| | | | | | (20 mg/kg) | | | | | |
| | | | Time in | ANOVA | Drug | 1.059 | 2.15 | 0.3712 | — | 9M |
| | | | Center | | | | | | | |
| | | | (sec) | | | | | | | |
| | Marble | MB | Marbles | ANOVA | Drug | 1.061 | 2.15 | 0.3708 | — | 9N |
| | Burying | | Buried | | | | | | | |
| | | | (no.) | | | | | | | |
| | Elevated | EPM | Time in | ANOVA | Drug | 1.018 | 2.15 | 0.3851 | — | 9O |
| | Plus | | Open | | | | | | | |
| | Maze | | Arms + | | | | | | | |
| | | | Center | | | | | | | |
| | | | (sec) | | | | | | | |
| | | | Time in | ANOVA | Drug | 0.895 | 2.15 | 0.4293 | — | data not |
| | | | Open | | | | | | | shown |
| | | | Arms (s) | | | | | | | |
| | | | Time in | ANOVA | Drug | 1.877 | 2.15 | 0.1873 | — | data not |
| | | | Closed | | | | | | | shown |
| | | | Arms (s) | | | | | | | |
| | | | Time in | ANOVA | Drug | 0.733 | 2.15 | 0.4971 | — | data not |
| | | | Center (s) | | | | | | | shown |
| | Novelty- | NSF | Fraction | Log-rank | Drug | — | — | 0.6032 | — | 9P |
| | Suppressed | | of mice | (Mantel- | | | | | | |
| | Feeding | | not eating | Cox) test | | | | | | |
| | | | in OF | | | | | | | |
| | | | Latency | ANOVA | Drug | 0.195 | 2.15 | 0.8249 | — | |
| | | | to feed in | | | | | | | |
| | | | OF (sec) | | | | | | | |
| | | | Fraction | Log-rank | Drug | — | — | 0.3747 | — | 9Q |
| | | | of mice | (Mantel- | | | | | | |
| | | | not eating | Cox) test | | | | | | |
| | | | in HC | | | | | | | |
| | | | Latency | ANOVA | Drug | 0.274 | 2.15 | 0.7641 | — | |
| | | | to feed in | | | | | | | |
| | | | HC (sec) | | | | | | | |
| | | | Change in | ANOVA | Drug | 2.183 | 2.15 | 0.1471 | — | 9R |
| | | | Weight (g) | | | | | | | |
| | | | Food | ANOVA | Drug | 0.845 | 2.15 | 0.4491 | — | 9S |
| | | | Eaten (g) | | | | | | | |

[symbol]

indicates data missing or illegible when filed

### Behavioral Methods

### Contextual Fear Conditioning (CFC)

A 3-shock CFC paradigm was administered as previously described (45, 46). CFC was conducted in chambers obtained from Med Associates (St. Albans, Vt.), with internal dimensions of approximately 20 cm wide×16 cm deep×20.5 cm high. The chambers had metal walls on each side, clear plastic front and back walls and ceilings, and stainless-steel bars on the floor. A house light (CM1820 bulb, 28v, 100 mA) mounted directly above the chamber provided illumination. Each chamber was located inside a larger, insulated, plastic cabinet that provided protection from outside light and noise. Each cabinet contained a ventilation fan that was operated during the sessions. A paper towel dabbed with lemon solution was placed underneath the chamber floor. Mice were held outside the experimental room in their home cages prior to testing and transported to the conditioning apparatus individually in standard mouse cages Chambers were cleaned with 70% EtOH between each set of mice. Mice were placed into the conditioning chamber and received shocks at 180 s, 240 s, and 300 s (2 s duration, 0.75 mA). Fifteen seconds after the last shock, mice were removed from the chamber. Overall, the training session lasted 317 s. During re-exposure, mice were placed in the conditioning chamber for 5 minutes and did not receive any shocks. All sessions were scored for freezing using FreezeFrame4.

### Forced Swim Test (FST)

The FST was administered as previously described (47). Briefly, mice were placed into clear plastic buckets 20 cm in diameter and 23 cm deep filled ⅔ of the way with 22° C. water. Mice were videotaped from the side for 6 min and were exposed to the swim test on 2 consecutive days Immobility time was scored by an experimenter blind to the experimental groups.

### Elevated Plus Maze (EPM)

Testing was performed as previously described (47). Briefly, the maze is a plus-cross-shaped apparatus consisting of four arms, two open and two enclosed by walls, linked by a central platform at a height of 50 cm from the floor. Mice were individually placed in the center of the maze facing an open arm and were allowed to explore the maze for 5 min. The time spent in and the number of entries into the open arms was used as an anxiety index. Videos were scored using ANY-maze behavior tracking software (Stoelting, Wood Dale, Ill.).

### Open Field (OF)

The OF assay was administered as previously described (47). Briefly, motor activity was quantified in 4 Plexiglas open field boxes 43×43 cm² (MED Associates, Georgia, Vt.). Two sets of 16 pulse-modulated infrared photobeams on opposite walls 2.5-cm apart recorded x-y ambulatory movements. Activity chambers were computer interfaced for data sampling at 100-ms resolution. The computer defined grid lines that dividing center and surround regions, with the center square consisting of four lines 11 cm from the wall.

### Marble Burying (MB)

The MB assay was conducted in a clean cage (10.5 in×5.5 in) containing soft pliable Beta Chip bedding (Northeastern Products Corp, Warrensburg, N.Y.). The cage contained 16 marbles set up in 4 rows of 4 across. Mice were given 30 minutes to explore and bury. At the end of the assay, the percentage of marbles buried was calculated.

### Novelty Suppressed Feeding (NSF)

Testing was performed as previously described (47). Briefly, the NSF testing apparatus consisted of a plastic box (50×50×20 cm). The floor of which was covered with approximately 2 cm of wooden bedding and the arena was brightly lit (approximately 1000 lux). Mice were food restricted for 24 h prior to testing. At the time of testing, a single pellet of food (regular chow) was placed on a white paper platform positioned in the center of the box. Each animal was placed in a corner of the box, and a stopwatch was immediately started. The latency of the mice to begin eating in the arena was recorded Immediately after the latency was recorded, the food pellet was removed from the arena. The mice were then placed back into their home cage. The latency to eat and the amount of food consumed in 5 min were measured (home cage consumption), followed by an assessment of post-restriction weight. A Kaplan-Meier survival analysis was used due to the lack of normal distribution of data. The Mantel-Cox log-rank test was used to evaluate differences between the experimental groups.

### Immunohistochemistry

Immunohistochemistry was performed as previously described (46). Mice were deeply anesthetized, and brains were fixed and extracted using transcardial perfusion. For c-fos immunohistochemistry, floating sections were used. Sections were first rinsed three times in 1×PBS and then blocked in 1×PBS with 0.5% Triton X-100 and 10% NDS for 2 hours at room temperature (RT). Incubation with primary antibodies was performed at 4° C. overnight (rabbit anti-c-fos, 1:5000, SySy, Göettingen, Germany, 226 003) in 1×PBS with 0.5% Triton X-100. Sections were then washed three times in 1×PBS and incubated with secondary antibody (Alexa 647 anti-rabbit, 1:500, Thermo Fisher Scientific, Waltham, Mass. A32733) for 2 hours at RT. Sections were then washed three times in 1×PBS, mounted on slides, and coverslipped with ProLong Gold (Invitrogen, Carlsbad, Calif.).

### Confocal Microscopy

Fluorescent confocal micrographs were captured with a Leica TCS SPE-II confocal microscope with LAS X software. Bilateral hippocampal sections were imaged throughout the rostro-caudal axis of the HPC using a 20× objective. Identification of hippocampal regions involved acquiring 6 dorsal and ventral sections per mouse brain slice at 20×. All individual panels were acquired at a thickness of 5 μm. Z-stack analysis was performed using the LAS X image browser to determine expression of c-fos. Expression levels of c-fos were compared across all sections using identical exposure conditions.

### Cell Quantification

An investigator blind to treatment groups used FIJI software to count c-fos⁺ immunoreactive cells in the DG, CA3, and CA1 throughout the entire rostrocaudal axis of the HPC. Cells were counted bilaterally. Number of c-fos+ cells is presented throughout the text.

### Western Blotting

Samples from mouse hippocampus and prefrontal cortex were dissected, snap frozen, and stored at −80° C. Brain tissue was homogenized in RIPA buffer with protease inhibitor (Thermo Fisher Scientific, Waltham, Mass.; Roche, Basel, Switzerland) using a motorized pestle mixer (Argos Technologies, Vernon Hills, Ill.) and spun at 1000 g×14 minutes to remove cell debris. Protein concentration was determined using a Bradford protein assay (Bio-Rad, Hercules, Calif.). 4× Laemmli buffer and β-mercaptoethanol (Bio-Rad, Hercules, Calif.) was added to the samples, which were then boiled at 90° C. for five minutes. 50 μg of each sample was separated by SDS/PAGE, and proteins were transferred onto PVDF membrane (GE Healthcare Life Sciences, Marlborough, Mass.). Membranes were then blocked in 1×TBS with 0.1% Tween-20 and 5% BSA for 1 hr at RT. Incubation with primary antibodies was performed at 4° C. overnight (rabbit anti-β-tubulin, 1:10000, Abc am, Cambridge, UK, ab6046; rabbit anti-NR2A, 1:1000, Alomone Labs, Jerusalem, Israel, AGC-002; rabbit anti-NR2B, 1:600, Alomone Labs, Jerusalem, Israel, AGC-003; mouse anti-GluR1, 1:1000, Millipore Sigma, Burlington, Mass., MAB2263) in 1×TBS with 0.1% Tween-20 and 5% BSA. Membranes were then washed three times in 1×TBS with 0.1% Tween-20 and incubated with secondary antibody (donkey anti-rabbit HRP, 1:5000, Thermo Fisher Scientific, Waltham, Mass., A16035; donkey anti-mouse HRP, 1:5000, Thermo Fisher Scientific, Waltham, Mass., A16011) for 2 hours at RT. Signal was revealed using chemiluminescent substrate (Thermo Fisher Scientific, Waltham, Mass.), visualized, and analyzed using ImageJ software. The ratio of signal from different proteins to α-tubulin was used to normalize the signal intensities and correct for loading and sampling errors. All data are reported as percentage of control.

### Results

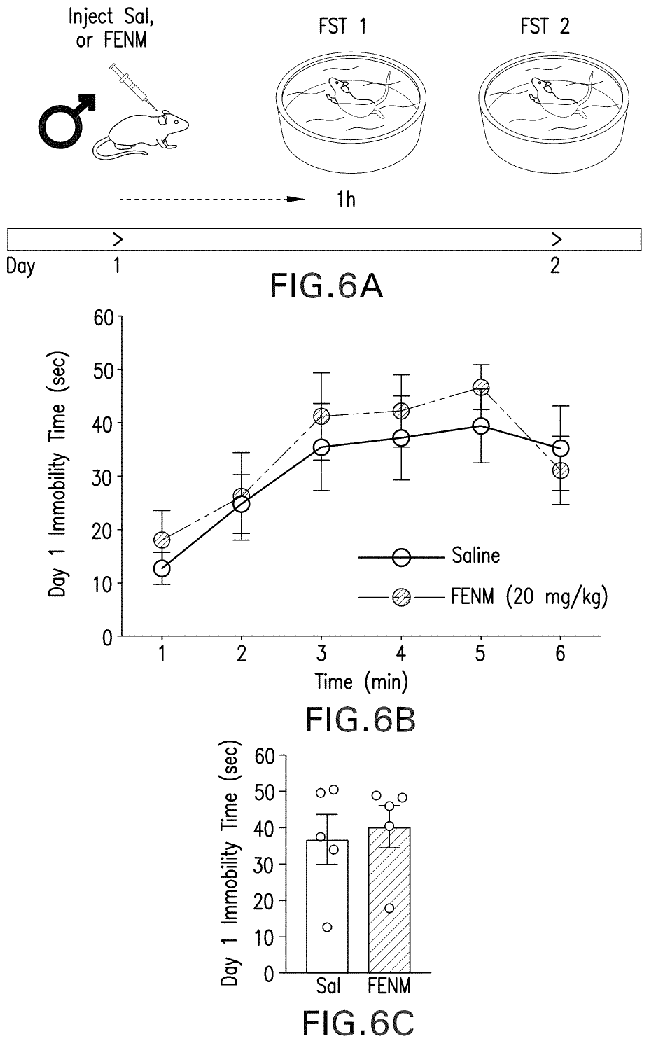

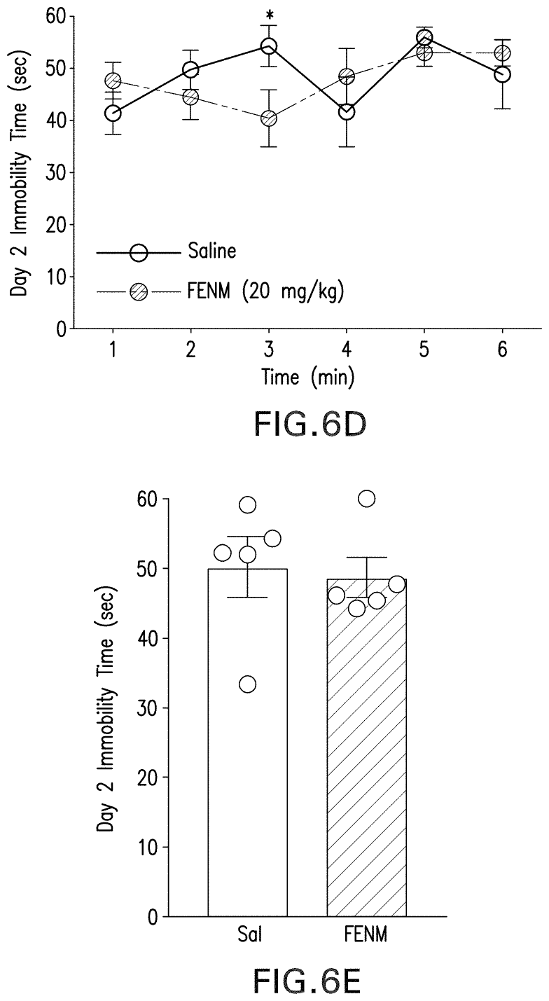

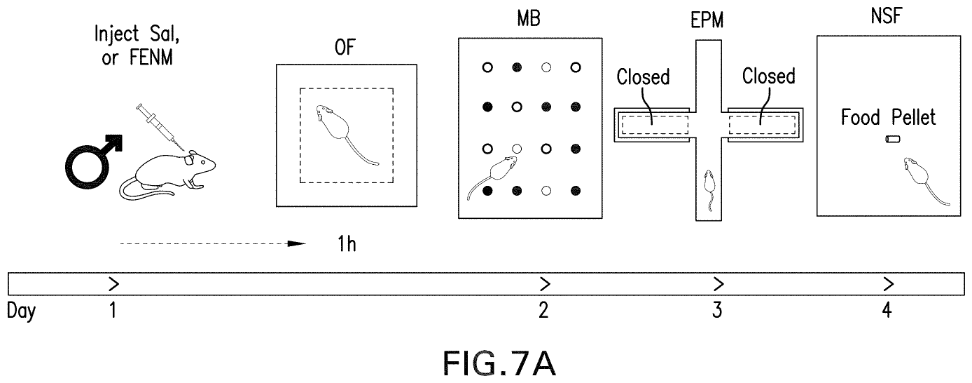

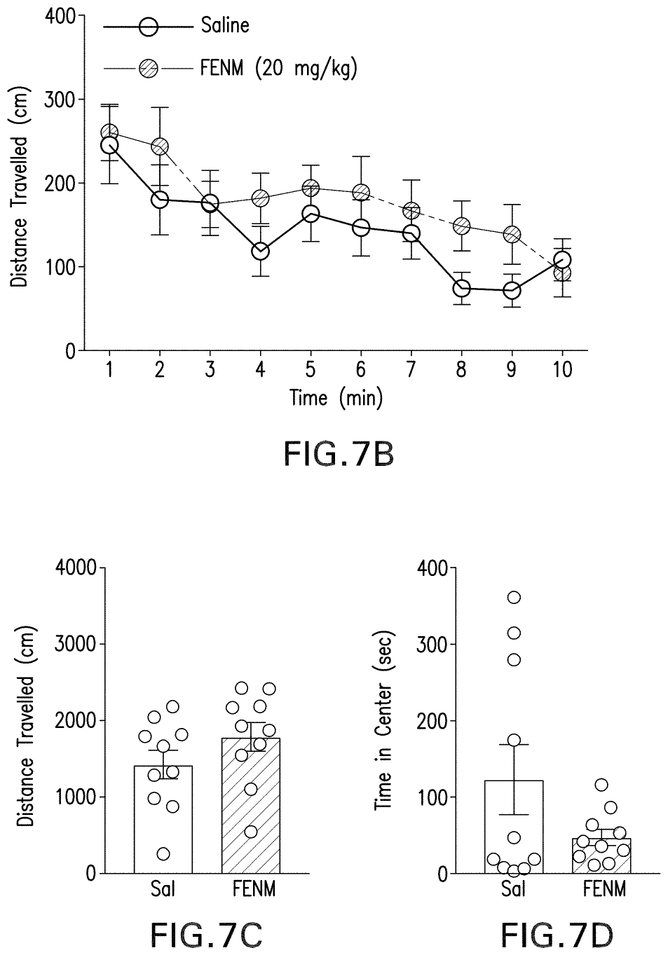

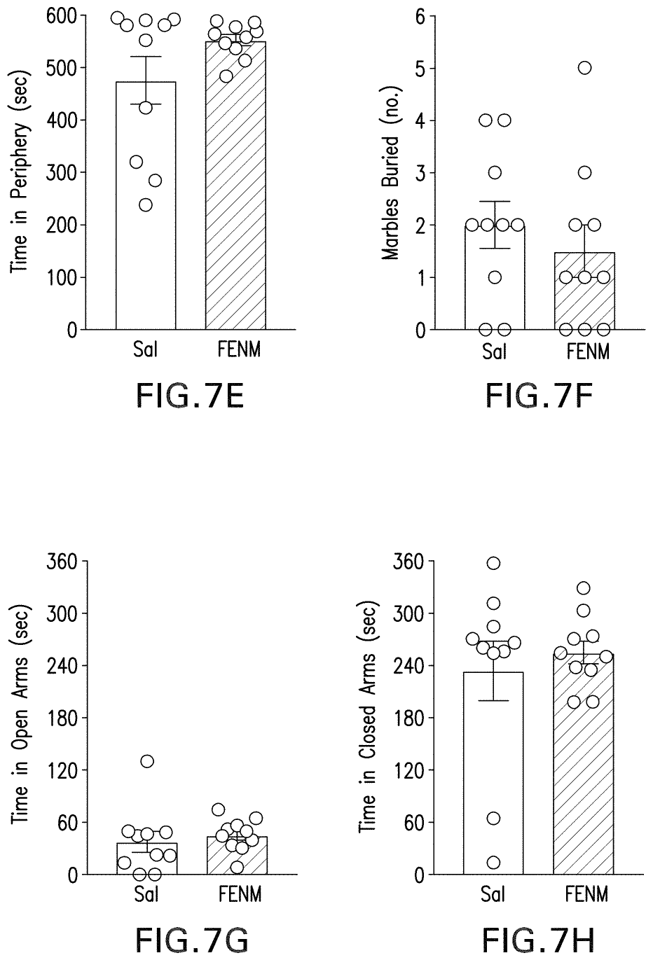

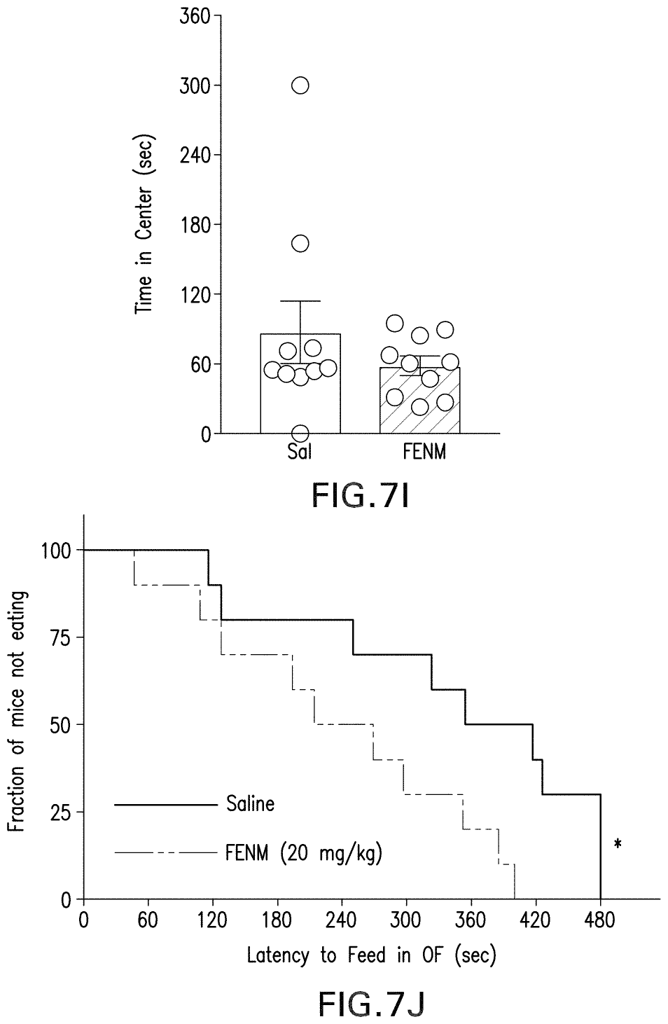

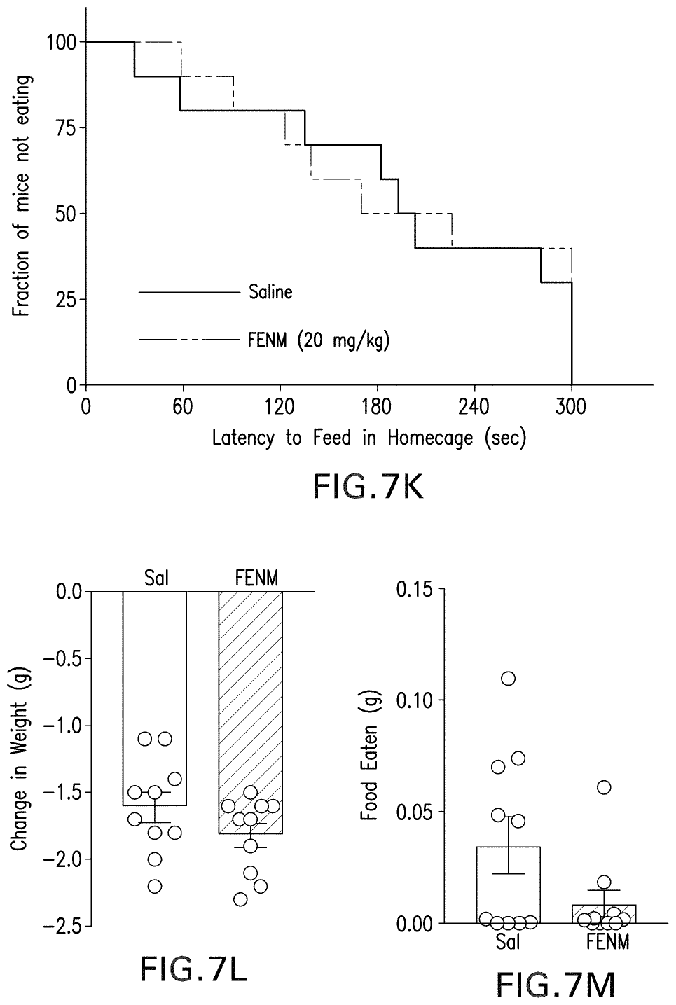

FENM does not Alter Behavioral Despair in Non-Stressed Mice
We first sought to test if FENM altered baseline depressive-like behavior. Saline or FENM (20 mg/kg) was administered 1 hour before the start of the FST (FIG. 6A). Both groups of mice had comparable immobility during day 1 (FIG. 6B-6C) and day 2 (FIG. 6D-6E) of the FST. These data indicate that FENM does not alter behavioral despair in non-stressed mice.
FENM does not Alter Anxiety-Like Behavior, but Decreases Hypophagia in Non-Stressed Mice
We next sought to test if FENM altered baseline anxiety-like behavior. Saline or FENM (20 mg/kg) was administered 1 hour before the start of the OF (FIG. 7A). Both groups of mice travelled a comparable distance in the OF (FIG. 7B-7C), spent a comparable amount of time in the center (FIG. 7D), and spent a comparable amount of time in the periphery (FIG. 7E). In the MB paradigm, FENM did not affect the number of marbles buried (FIG. 7F). In the EPM, both groups of mice spent a comparable amount of time in the open arms (FIG. 7G), closed arms (FIG. 7H), and center (FIG. 7I). In the NSF paradigm, FENM administration significantly decreased the latency to approach the pellet in the NSF arena when compared with saline administration (FIG. 7J). However, FENM did not alter the latency to eat in the homecage (FIG. 7K). FENM did not impact the amount of weight loss in the NSF paradigm (FIG. 7L). Both groups of mice ate a comparable amount of food in the home cage (FIG. 7M). These data indicate that FENM does not alter anxiety-like behavior, but decreases hypophagia in non-stressed mice.

### FENM is a Novel Prophylactic Against Learned Fear and Stress-Induced Depressive-Like Behavior

We have previously reported that (R,S)-ketamine administration 1 week before stress results in prophylactic efficacy in 12956/SvEv mice (Brachman et al., 2016). Here, we sought to determine if FENM was an effective prophylactic since its mechanism of action is similar to (R,S)-ketamine in that they both are NMDAR antagonists. Saline, (R,S)-ketamine (30 mg/kg), or FENM (10, 20, or 30 mg/kg) was administered 1 week prior to CFC (FIG. 1A). Freezing was comparable between all groups during CFC training (FIG. 1B). During CFC re-exposure, (R,S)-ketamine (30 mg/kg) and FENM (20 and 30 mg/kg) administration decreased fear expression (FIG. 1C-1D). On day 1 of the FST 1, all groups exhibited comparable immobility time (FIG. 1E). On day 2 of the FST, all drugs and doses tested significantly reduced immobility time when compared with saline mice (FIG. 1F-1G). These data indicate that like (R,S)-ketamine, FENM attenuates learned fear and decreases stress-induced depressive-like behavior when administered as a prophylactic.

We next assayed stress-induced anxiety-like behavior. In the OF, (R,S)-ketamine and FENM did not alter distance travelled or time spent in the center of the arena (FIG. 1H-1J). In the MB task, all groups buried a comparable number of marbles (FIG. 1K). In the EPM, (R,S)-ketamine and FENM did not alter distance travelled or time spent the open arms and center of the maze (FIG. 1L-1M). In the NSF, (R,S)-ketamine and FENM did not alter latency to feed in the open arena (FIG. 1N). Moreover, weight loss and food eaten in the homecage were comparable between all drug groups (FIG. 1O-1P). These data indicate that while FENM is not a robust prophylactic against stressed-induced anxiety-like behavior, it is a robust prophylactic against learned fear and stress-induced depressive-like behavior.

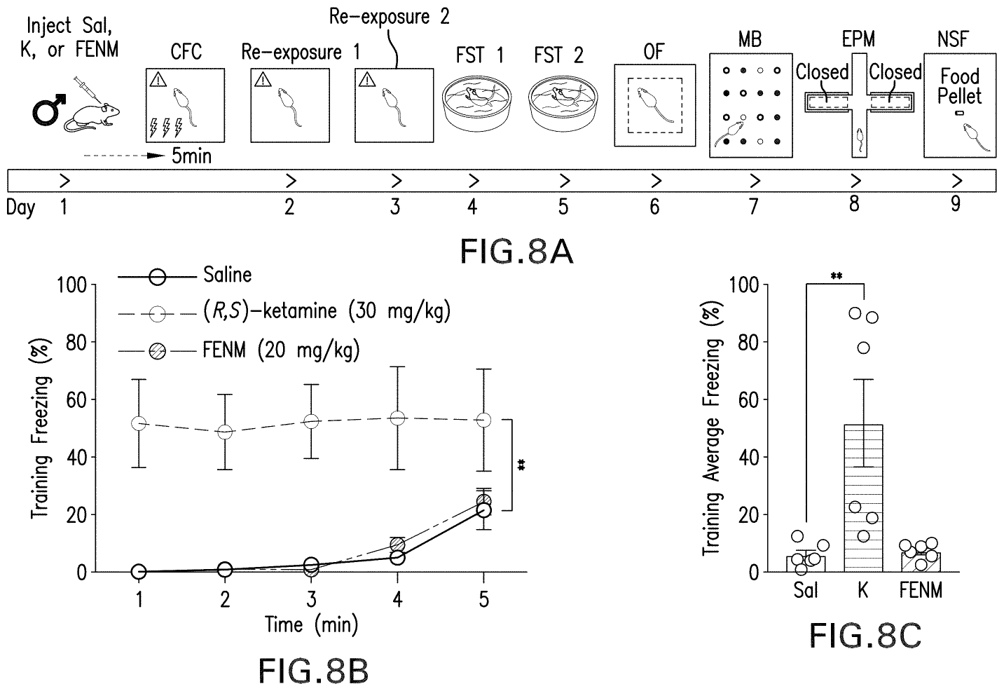

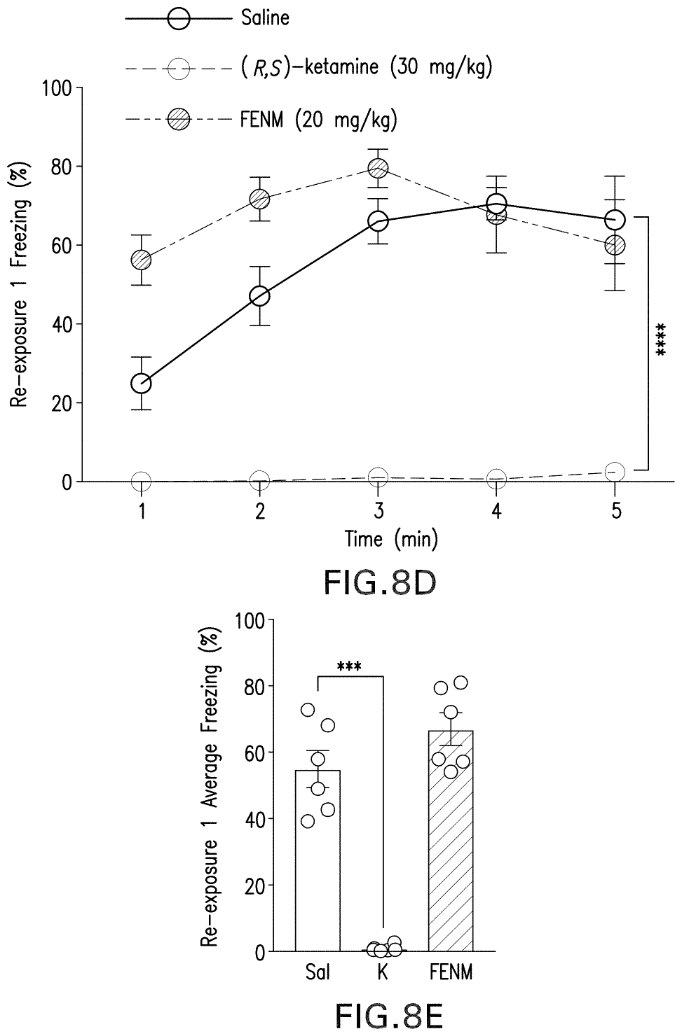

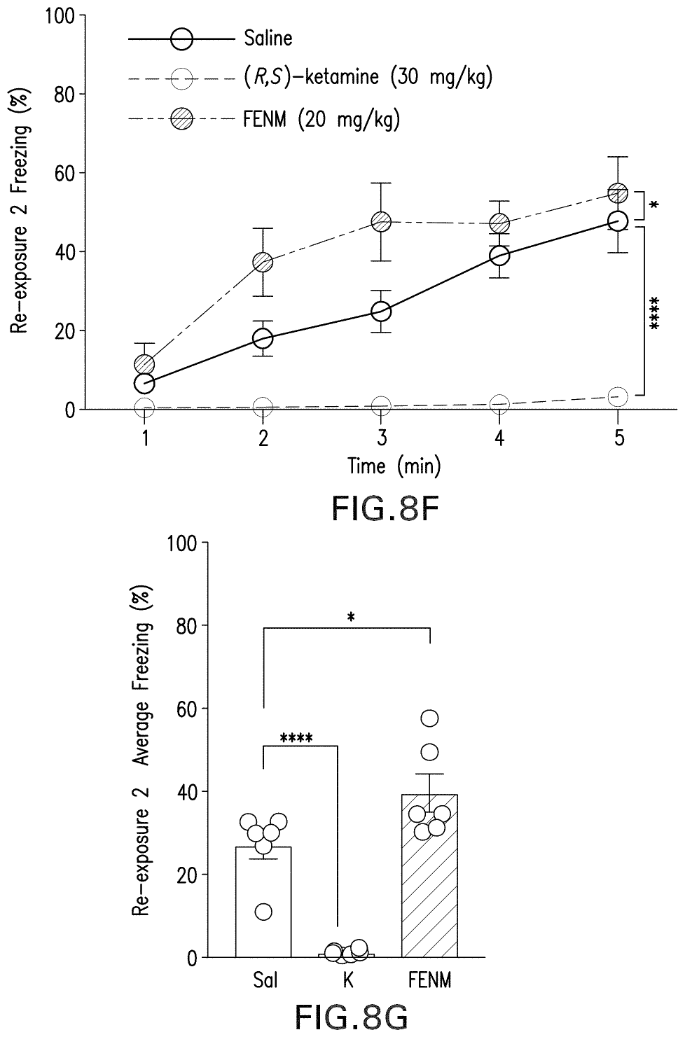

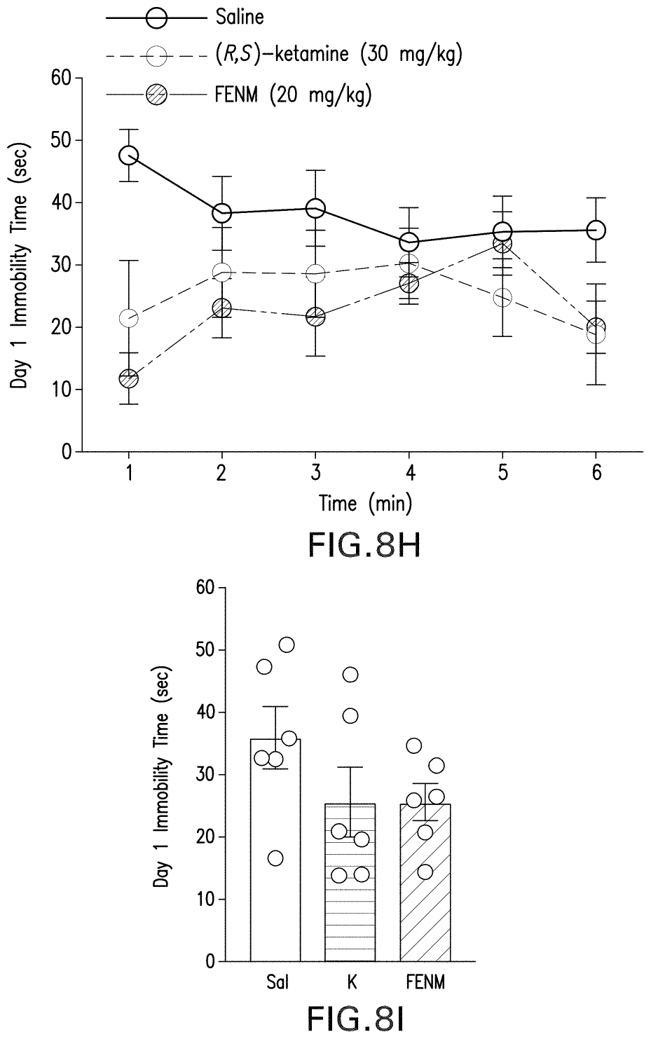

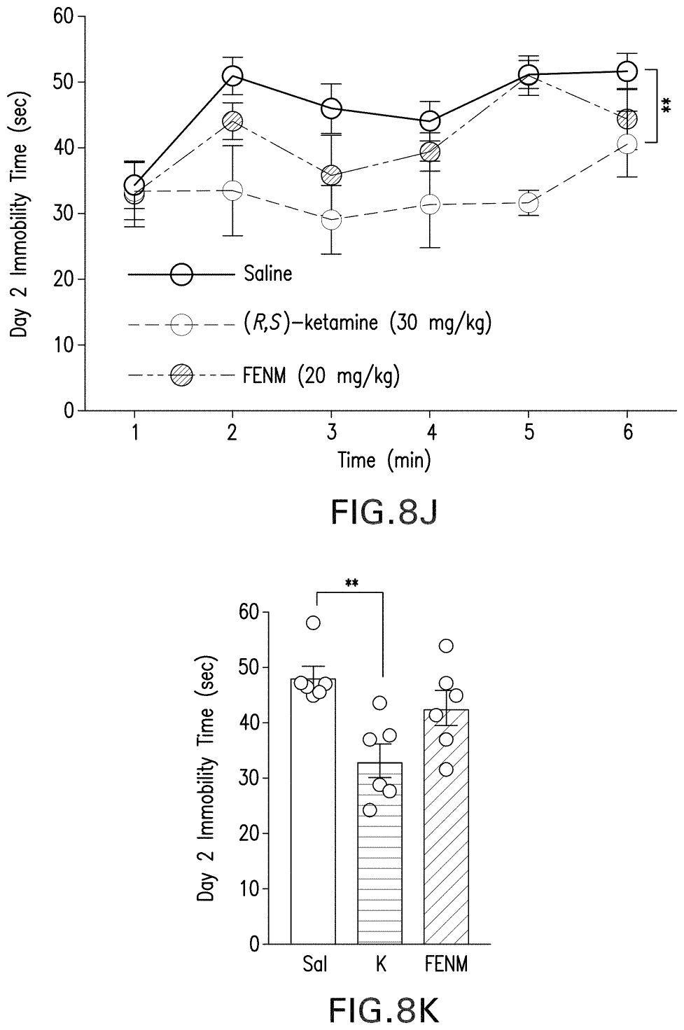

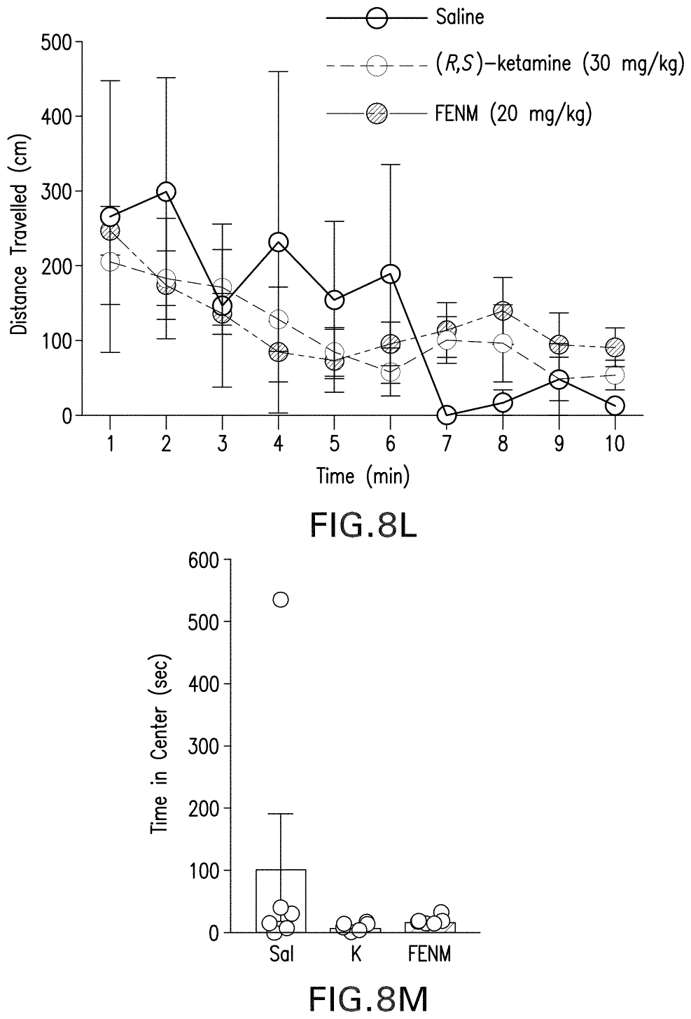

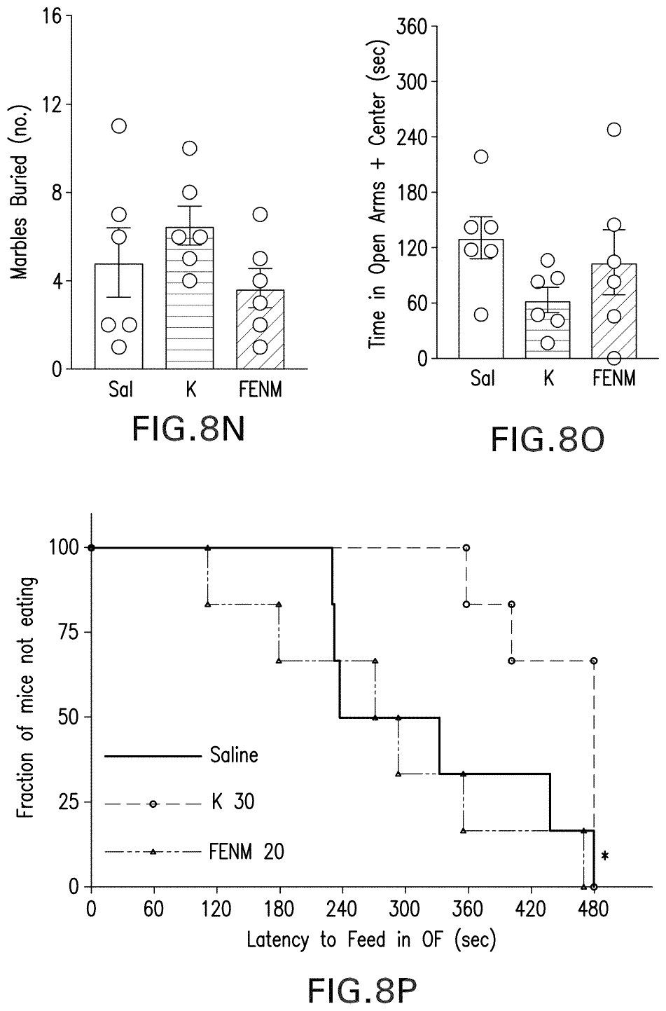

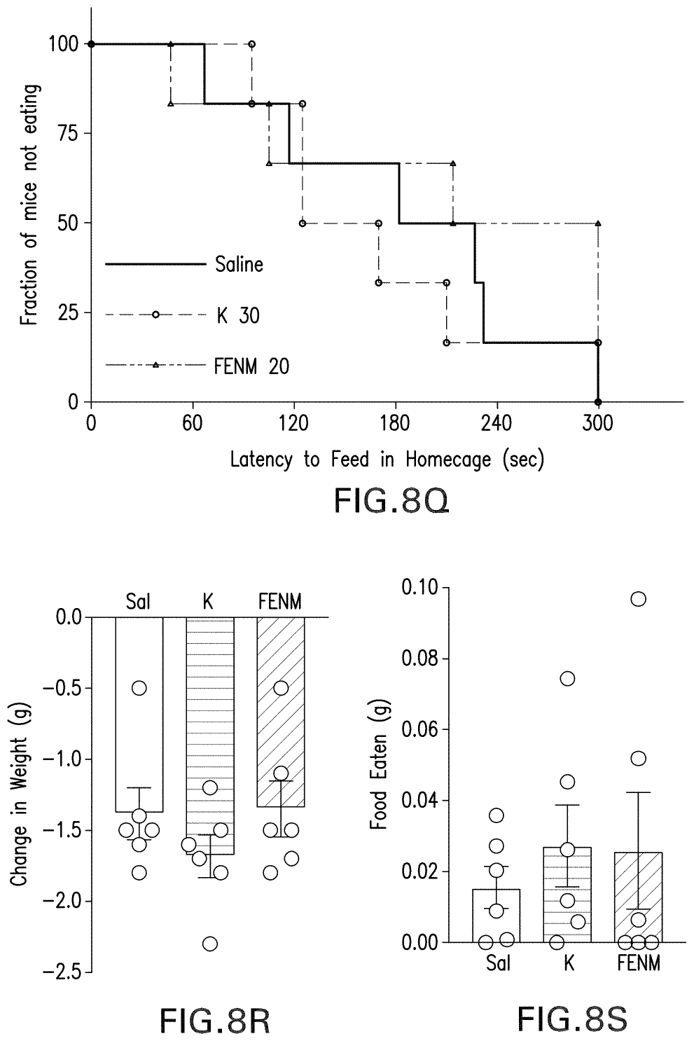

FENM is not a Prophylactic Against Stress-Induced Depressive-Like Behavior when Administered 5 Minutes Before Stress
We next sought to determine if FENM was effective as a prophylactic when administered with a shorter time interval before CFC. Saline, (R,S)-ketamine (30 mg/kg), or FENM (20 mg/kg) was administered 5 min prior to CFC (FIG. 8A). During CFC training, (R,S)-ketamine significantly increased freezing when compared with saline and FENM (FIG. 8B-8C). This increase in freezing behavior during training is most likely due to the psychotropic qualities of (R,S)-ketamine, resulting in increased immobility.

During CFC re-exposure 1, (R,S)-ketamine, but not FENM significantly decreased fear expression (FIG. 8D-8E). During CFC re-exposure 2, (R,S)-ketamine significantly decreased fear expression when compared with saline (FIG. 8F-8G). However, FENM significantly increased fear expression when compared with saline. In the (R,S)-ketamine-treated mice, the decrease in freezing behavior during testing is most likely due to the (R,S)-ketamine being active during CFC training, resulting in ineffective encoding of the CFC context.

On day 1 of the FST 1, all groups exhibited comparable immobility time (FIG. 8H-8I). On day 2 of the FST, (R,S)-ketamine, but not FENM, significantly decreased immobility time when compared with saline (FIG. 8J-8K). In the OF, (R,S)-ketamine and FENM did not alter distance travelled or time spent in the center of the arena (FIG. 8L-8M). In the MB task, all groups buried a similar number of marbles (FIG. 8N). In the EPM, all groups spent a comparable amount of time in the open arms and center of the maze (FIG. 8O). In the NSF, (R,S)-ketamine, but not FENM, increased the latency to feed in the open arena (FIG. 8P). However, all groups fed at a comparable latency in the homecage (FIG. 8Q). Moreover, body weight loss and food eaten in the homecage were comparable between all drug groups (FIG. 8R-8S). These data indicate that neither (R,S)-ketamine or FENM is a robust prophylactic against stress when administered immediately (e.g., 5 min) prior to a stressor.

FENM is a Novel Antidepressant when Administered Following Stress
Since FENM is an effective prophylactic when administered 1 week before stress, we next sought to determine if FENM may be effective as an antidepressant when administered following stress. Saline, (R,S)-ketamine (30 mg/kg), or FENM (10, 20, or 30 mg/kg) was administered 5 minutes after CFC (FIG. 2A). The interval between CFC training and re-exposure was 5 days as was previously described in FIG. 1. Freezing was comparable between all groups during CFC training (FIG. 2B) and during context re-exposure (FIG. 2C-2D). On day 1 of the FST 1, all groups exhibited comparable immobility time (FIG. 2E). On day 2 of the FST, all drugs and doses tested significantly reduced immobility time when compared with saline mice (FIG. 2F-2G), indicating both (R,S)-ketamine (30 mg/kg) or FENM are effective at decreasing stress-induced depressive-like behavior.

We next assayed stress-induced anxiety-like behavior. In the OF, all groups of mice travelled a comparable distance travelled and spent a comparable amount of time in the center of the arena (FIG. 2H-2J). In the MB task, FENM (20 and 30 mg/kg), but not (R,S)-ketamine decreased the number of marbles buried (FIG. 2K). In the EPM, (R,S)-ketamine and FENM did not alter distance travelled or the time spent in the open arms and center of the maze (FIG. 2L-2M). In the NSF, (R,S)-ketamine and FENM did not alter the latency to feed in the open arena (FIG. 2N). Moreover, latency to feed in the home cage, body weight loss, and food eaten in the home cage were comparable between all drug groups (FIG. 2O-2P). These data indicate that in addition to being a robust prophylactic, FENM can be administered immediately following stress to decrease stress-induced depressive-like behavior.

We next sought to determine if FENM was still effective if administered following a stressor, but if the interval between stress and behavioral testing was decreased. Saline, (R,S)-ketamine (30 mg/kg), or FENM (20 mg/kg) was administered 5 minutes after CFC as described in FIG. 2 (FIG. 3A). However, here, CFC context re-exposure occurred 1 day following CFC training as opposed to 5 days. Freezing was comparable between all groups during CFC training (FIG. 3B-3C). During CFC re-exposures 1 and 2, all groups of mice exhibited comparable freezing (FIG. 3D-3G). On day 1 of the FST, all groups exhibited comparable immobility time (FIG. 3H-31). On day 2 of the FST, FENM, but not (R,S)-ketamine significantly reduced immobility time when compared with saline mice (FIG. 3J-3K).

In the OF, all groups of mice travelled a comparable distance travelled and spent a comparable amount of time in the center of the arena (FIG. 3L-3M). In the MB task, all groups of mice buried a comparable number of marbles (FIG. 3N). In the EPM, all groups spent a comparable amount of time in the open arms and center of the maze (FIG. 3O). In the NSF, (R,S)-ketamine and FENM did not alter the latency to feed in the open arena (FIG. 3P) or in the home cage (FIG. 3Q). Moreover, latency to feed in the home cage, body weight loss, and food eaten in the home cage were comparable between all groups (FIG. 3U-3V). These data indicate that FENM, but not (R,S)-ketamine, is effective against stress-induced depressive-like behavior.

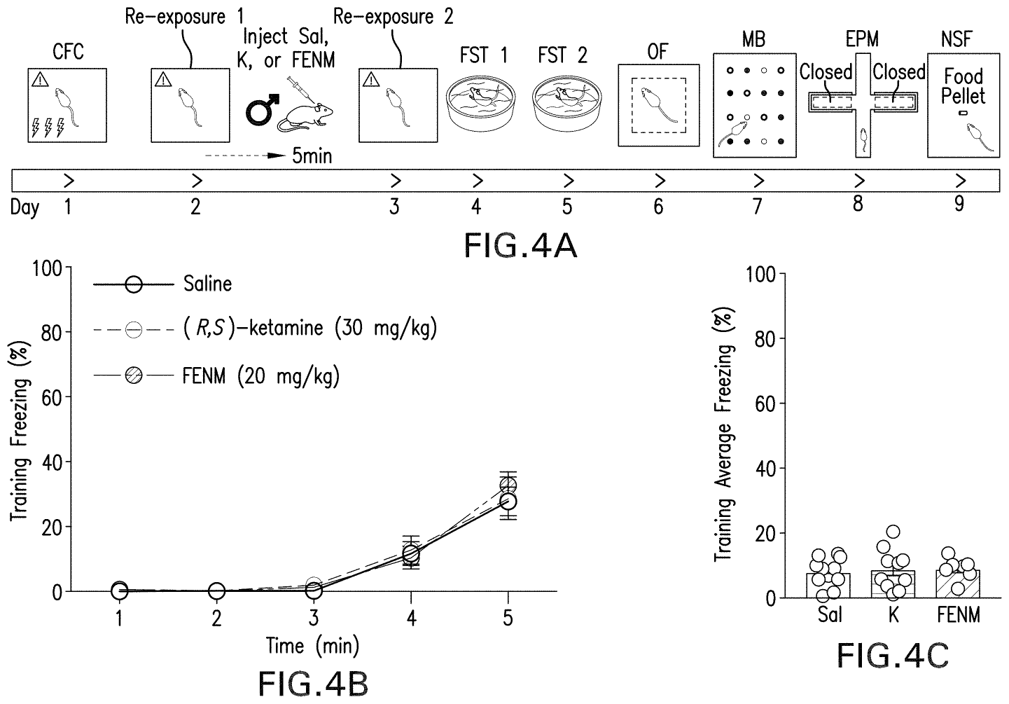

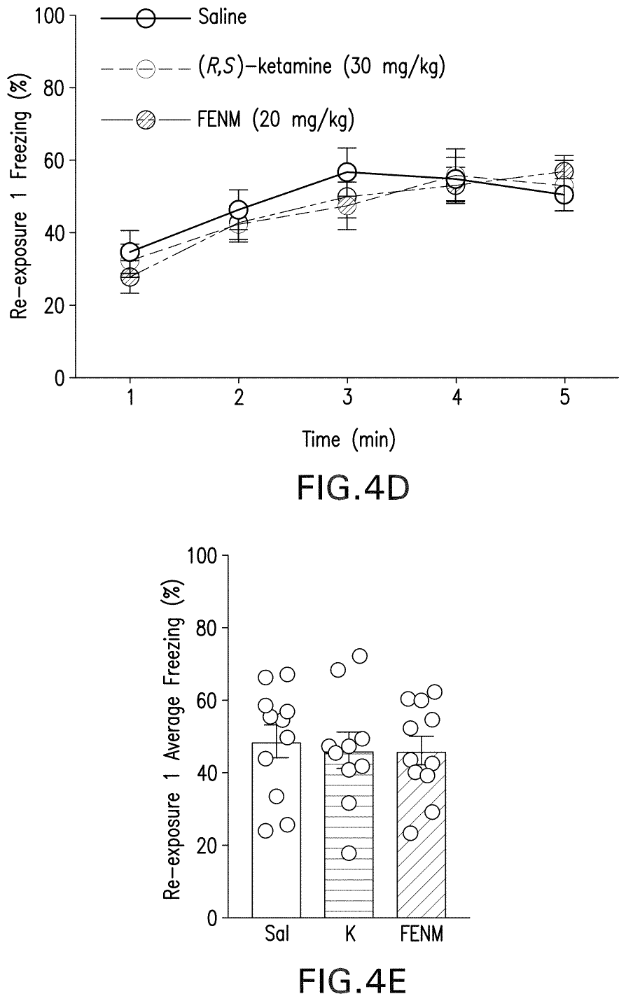

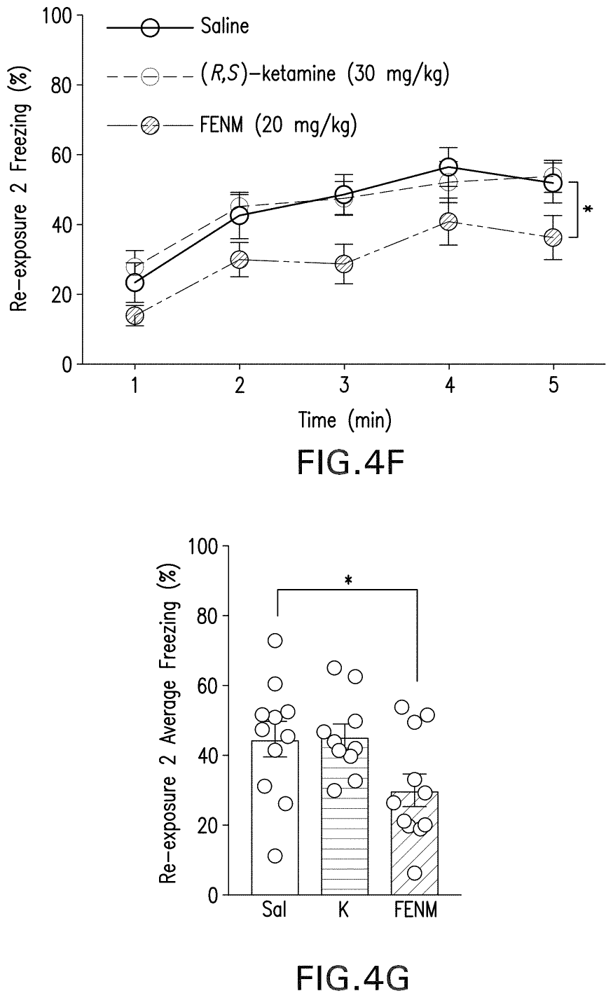

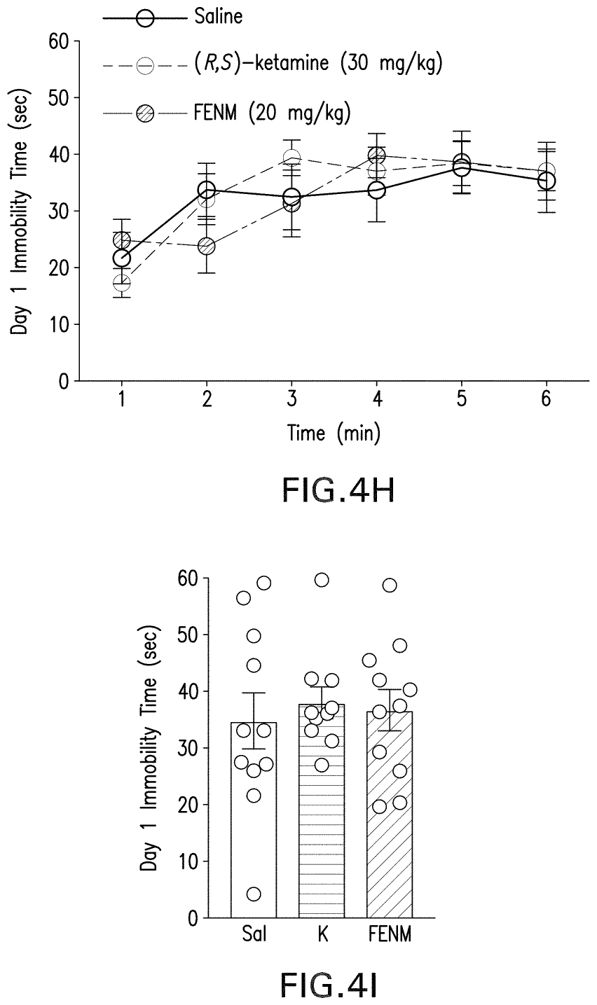

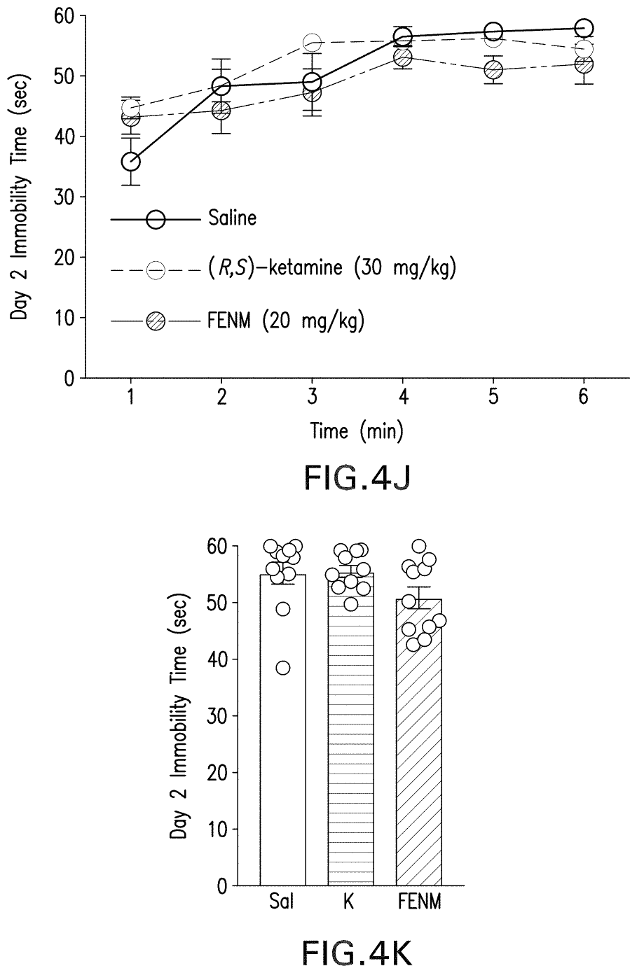

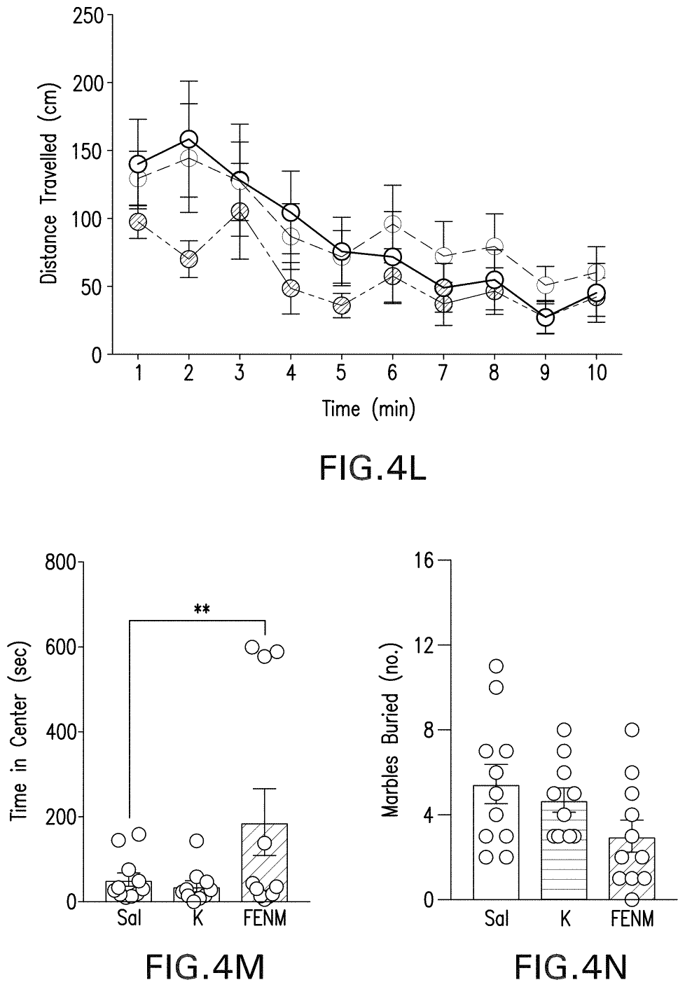

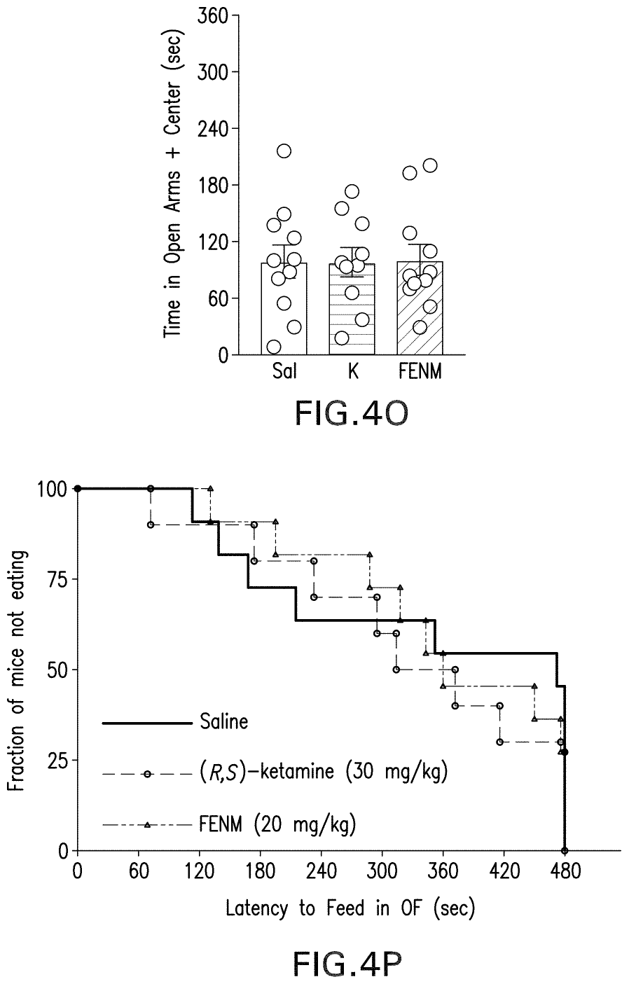

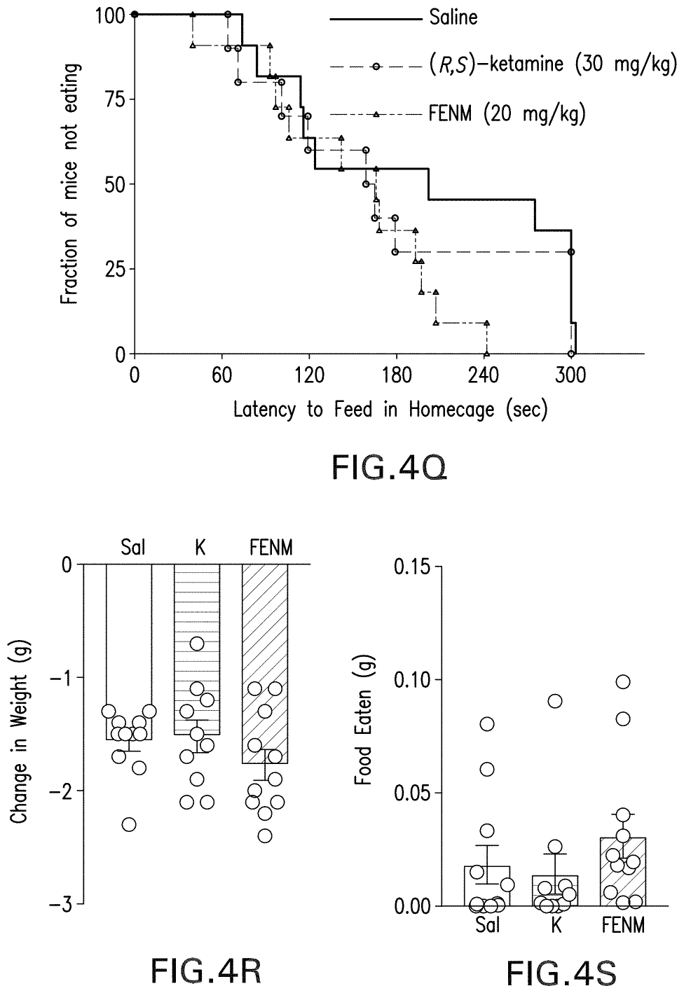

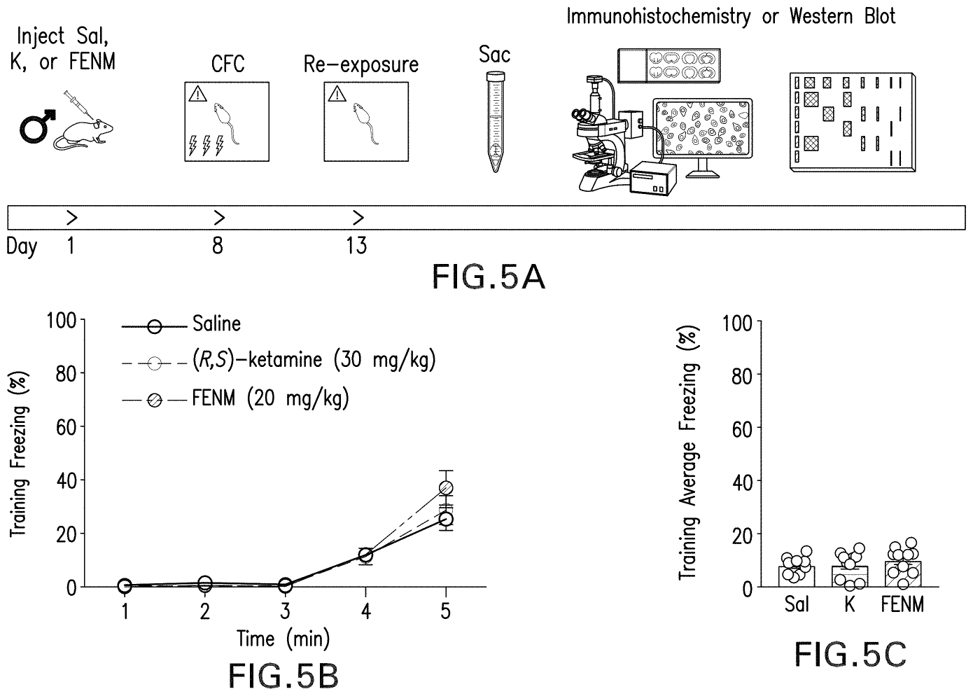

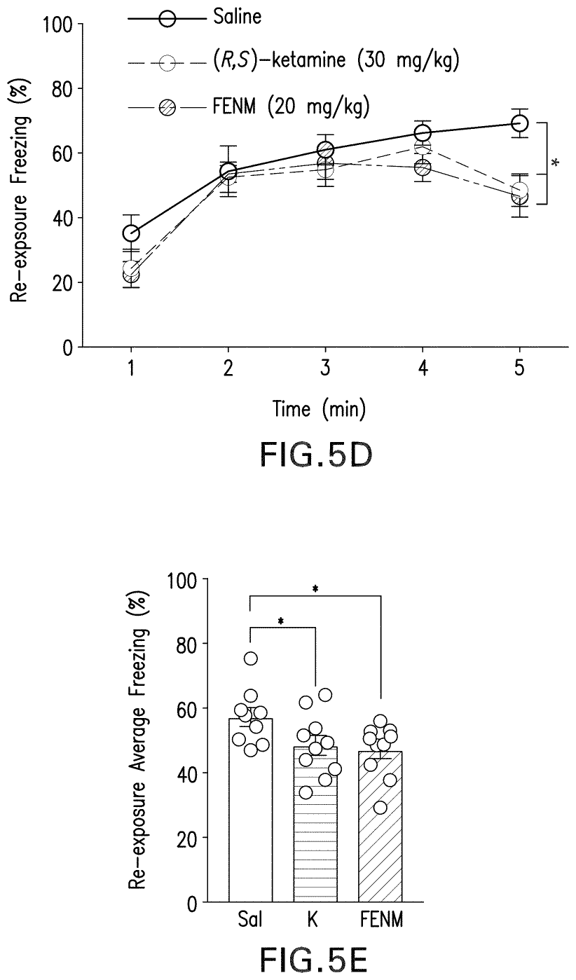

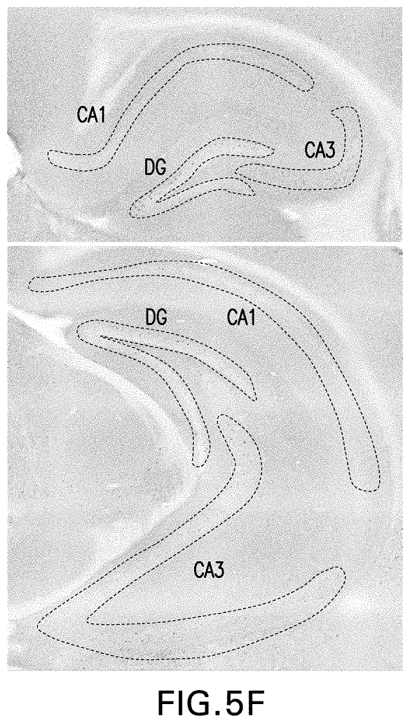

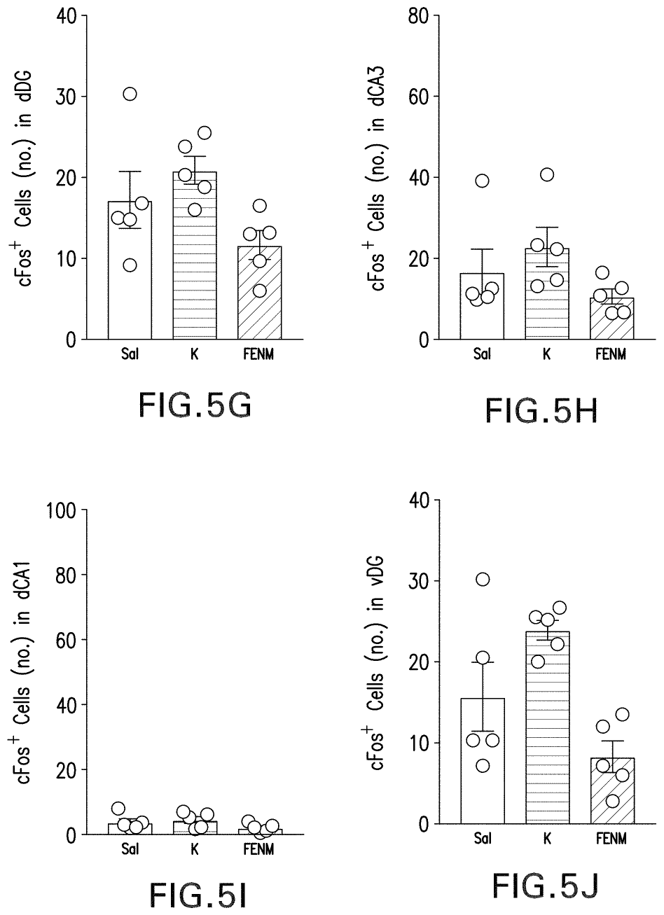

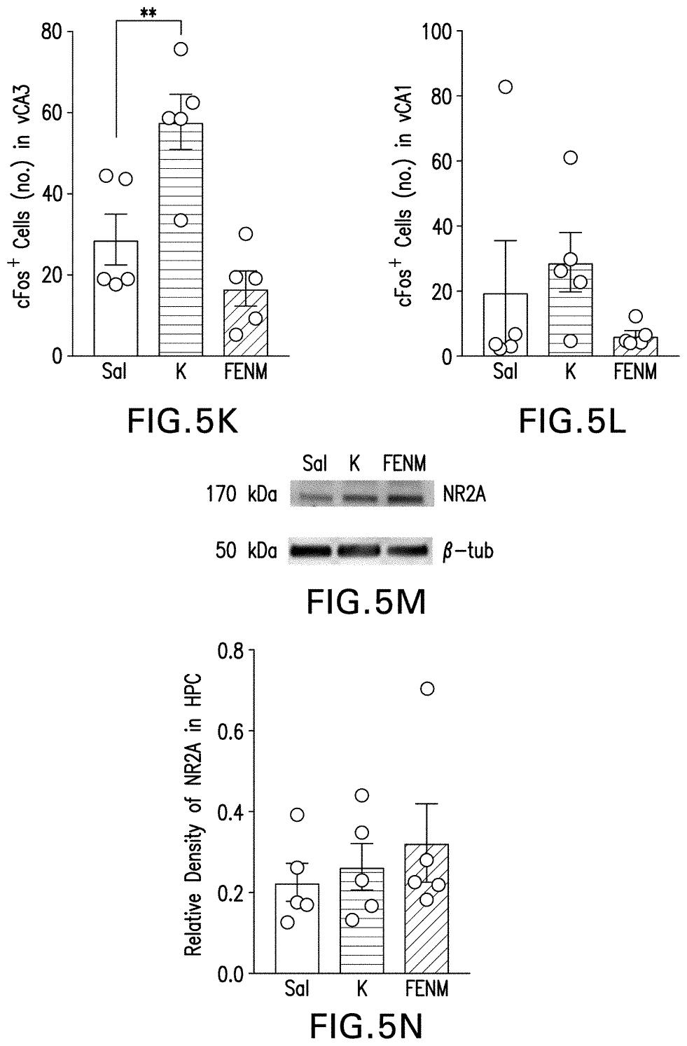

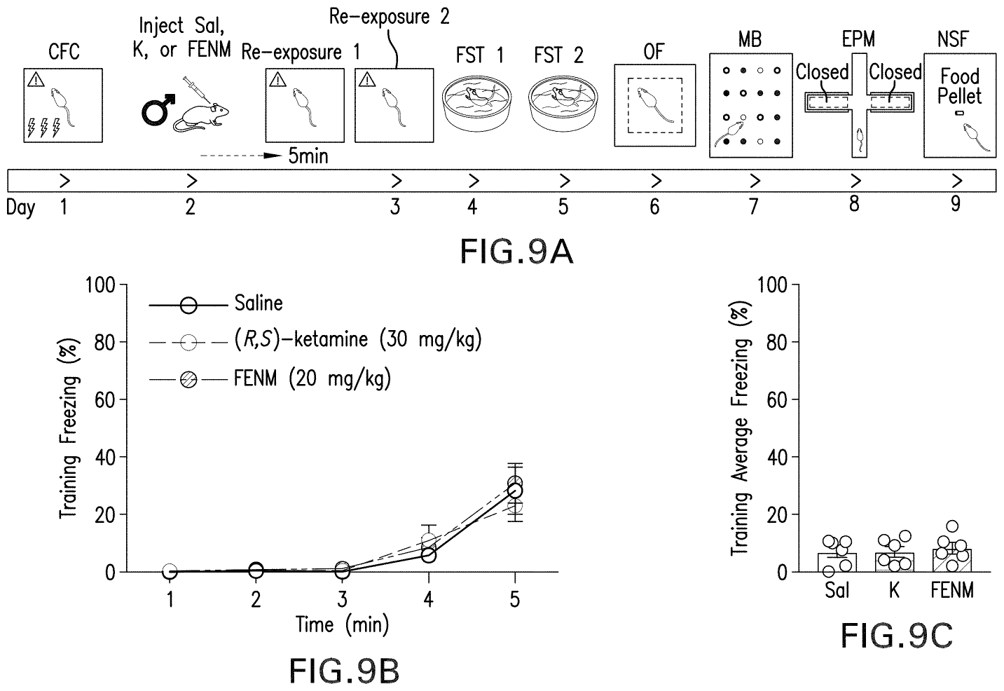

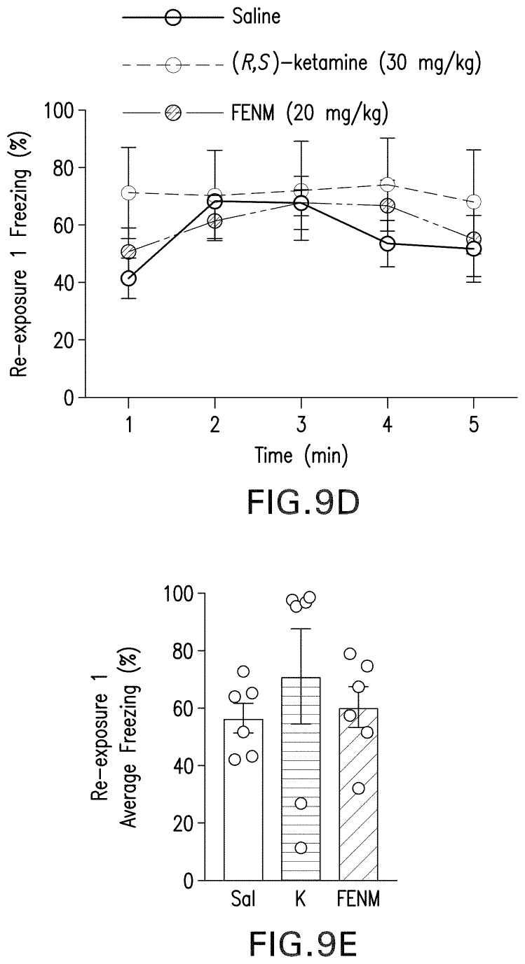

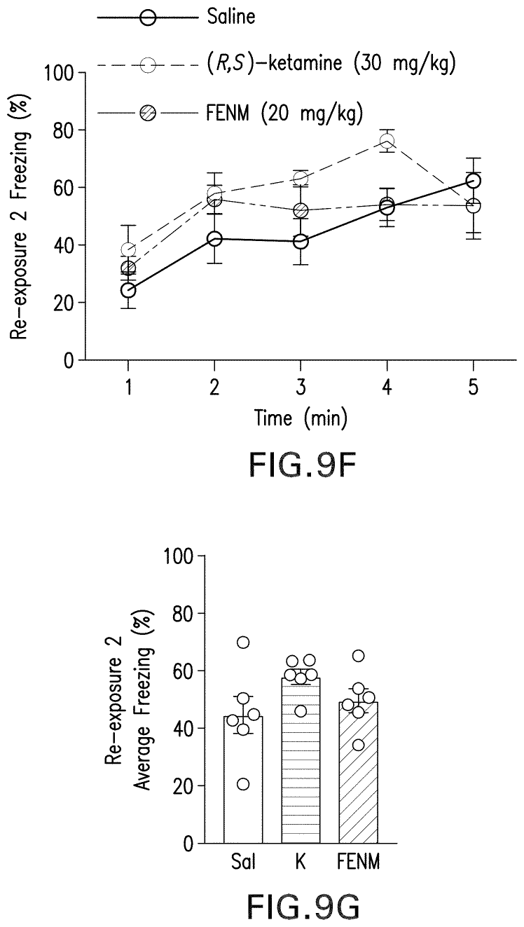

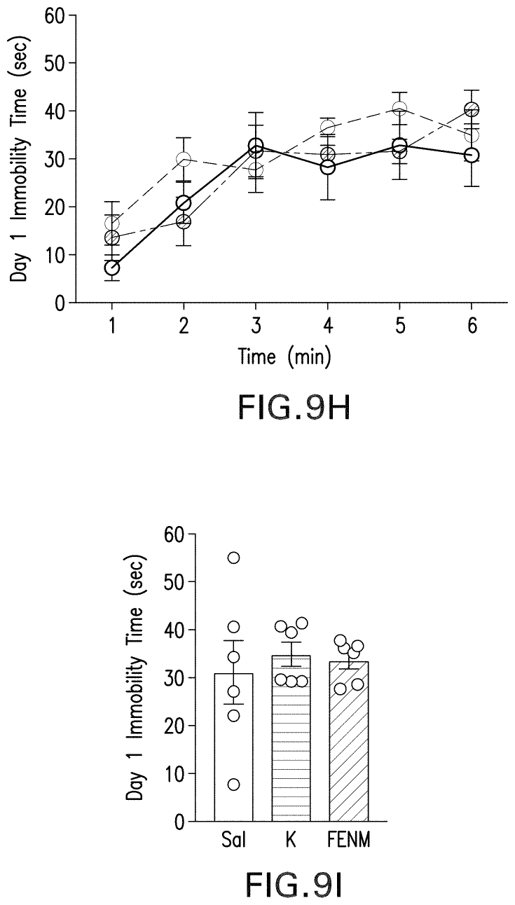

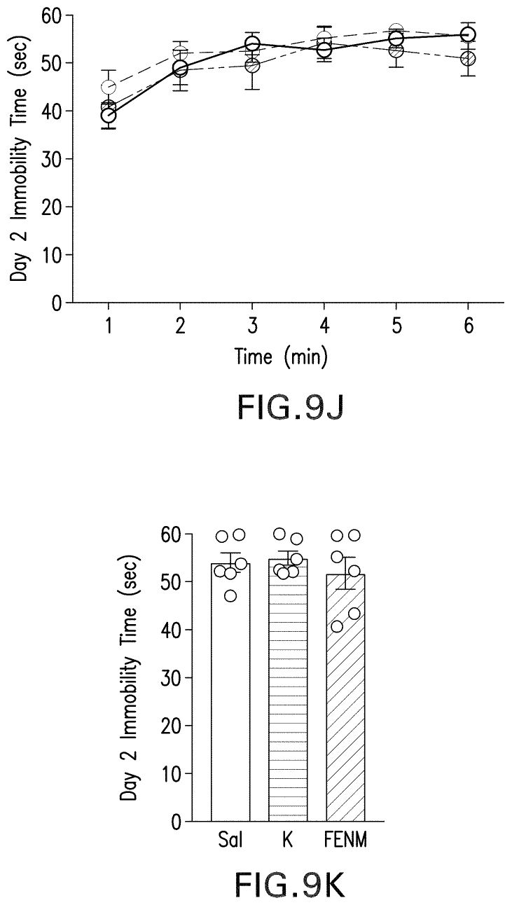

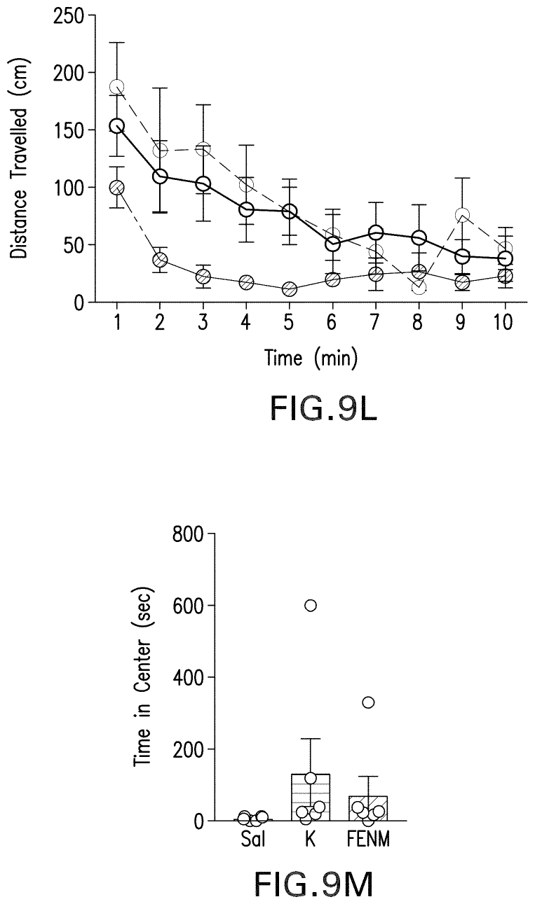

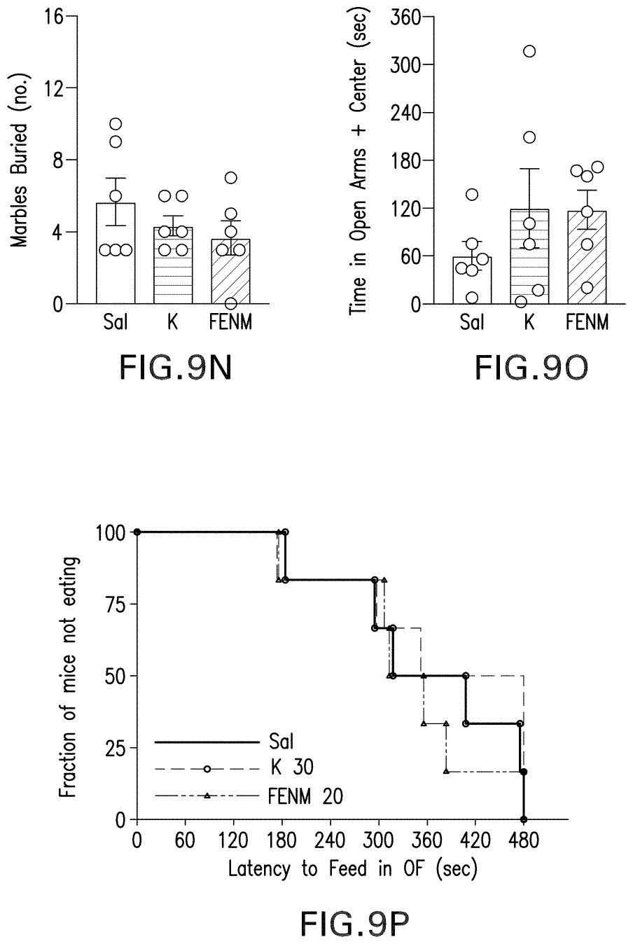

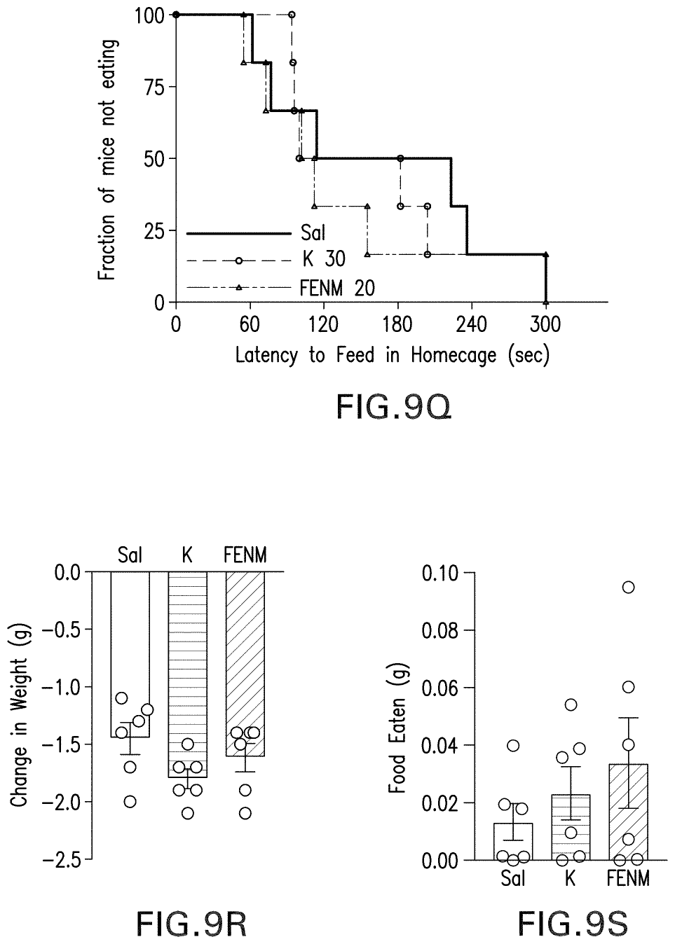

FENM is not Effective when Administered Immediately Prior to an Extinction Trial
To determine if FENM could facilitate extinction training, mice were first administered CFC (FIG. 9A). Twenty four hours later, saline, (R,S)-ketamine (30 mg/kg), or FENM (20 mg/kg) was administered 5 minutes prior to context re-exposure 1. During CFC training (FIG. 9B-9C), re-exposure 1 (FIG. 9D-9E), and re-exposure 2 (FIG. 9F-9G), freezing was comparable between all groups. During day 1 (FIG. 9H-9I) and day 2 (FIG. 9J-9K) of the FST, all groups exhibited comparable immobility time. In all anxiety tests, FENM and (R,S)-ketamine did not alter behavior when compared to saline (FIG. 9L-9S). These data indicate that (R,S)-ketamine and FENM are not effective at facilitating context extinction when administered prior to the first context re-exposure.
FENM is Effective in Attenuating Learned Fear when Administered Following an Extinction Trial
We then sought to determine if FENM could facilitate extinction training if administered following an extinction trial. All mice were administered CFC and 24 hours later, were administered context re-exposure 1 (FIG. 4A). Five minutes later, saline, (R,S)-ketamine (30 mg/kg), or FENM (20 mg/kg) was administered. Context re-exposure 2 occurred 24 hours later. During CFC training (FIG. 4B-4C) and re-exposure 1 (FIG. 4D-4E), freezing was comparable between all groups. Interestingly, during re-exposure 2, FENM, but not (R,S)-ketamine, significantly decreased fear expression when compared with saline (FIG. 4F-4G). During day 1 (FIG. 4H-4I) and day 2 (FIG. 4J-4K) of the FST, all groups exhibited comparable immobility time. In all anxiety tests, FENM and (R,S)-ketamine did not alter behavior when compared to saline (FIG. 4L-4S) with the exception of the time spent in the center of the OF (FIG. 4M). FENM significantly increased the time spent in the center when compared with saline administration. Overall, these data suggest that FENM is not effective when administered prior to extinction training, but may be effective for attenuating fear expression when administered following a context exposure.
FENM does not Alter Hippocampal Activity During Re-Exposure
Finally, we aimed to investigate cellular and/or molecular mechanisms that may contribute to the prophylactic and/or antidepressant actions of FENM. Previously, we showed that prophylactic (R,S)-ketamine may alter fear memory retrieval to attenuate learned fear (12). In order to determine whether FENM also alters hippocampal memory traces, male mice were administered a single dose of saline, (R,S)-ketamine (30 mg/kg), or FENM (20 mg/kg) one week prior to 3-shock CFC. Five days later, mice were re-exposed to the training context and sacrificed one hour later. We then used immunohistochemistry to quantify expression of the immediate early gene c-fos (FIG. 5A). Both (R,S)-ketamine and FENM did not alter behavior during CFC training but significantly reduced freezing upon re-exposure when compared to saline controls (FIG. 5B-5E). The number of c-fos⁺ neurons was comparable across all groups in all subregions of the dorsal hippocampus, as well as in the ventral DG and ventral CA1 (FIG. 5F-5J, 5L). Prophylactic (R,S)-ketamine, but not FENM, significantly increased c-fos expression in the ventral CA3 of the hippocampus, consistent with previous studies (12) (FIG. 5K). These data indicate that although both drugs attenuate learned fear, FENM, unlike (R,S)-ketamine, does not alter activity in the vCA3 during fear memory recall.

Because FENM, similarly to (R,S)-ketamine, acts as an NMDAR antagonist, we hypothesized that the drug may alter expression of either NMDAR or AMPAR subunits in the hippocampus. To determine whether NMDAR or AMPAR expression was altered at the time of fear memory recall, we used Western blotting to quantify protein expression after fear memory recall (FIG. 5A). We found that NR2A expression was comparable across all groups in the hippocampus, indicating that neither FENM nor (R,S)-ketamine alters total expression of the NR2A NMDAR subunit in the hippocampus during re-exposure (FIG. 5M-5N).

### Discussion

Here, we hypothesized that FENM could be an effective prophylactic and/or antidepressant against stress-related psychiatric disorders. We tested several different doses either in the absence of stress or at varying time points before and after stress. At baseline, FENM did not alter depressive-like behavior but reduced stress-induced hyponeophagia. FENM attenuated learned fear and prevented depressive-like behavior when administered 7 days, but not 5 minutes, before stress. When administered as an antidepressant 5 minutes after stress, FENM effectively reduced depressive- and perseverative behavior. Moreover, when administered 5 minutes after, but not 5 minutes before, context re-exposure, FENM decreased fear expression. FENM did not alter hippocampal activity during fear memory retrieval or alter NMDAR expression in the hippocampus. A complete summary of our behavioral results comparing (R,S)-ketamine and FENM can be found in Table 2.

TABLE 2
Summary of behavioral results

| | | | Behavior | | | |
|---|---|---|---|---|---|---|
| | | Dose | | Depressive- | Anxiety- | |
| Experiment | Compound | (mg/kg) | Fear | like | like | Hypophagia |
| Baseline | FENM | 20 | N/A | — | N/A | N/A |
| Antidepressant | | | | | | |
| Baseline | FENM | 20 | N/A | N/A | — | ↓ |
| Anxiolytic | | | | | | |
| Prophylactic | (R,S)- | 30 | ↓ | ↓ | — | — |
| (7 days before | ketamine | | | | | |
| stress) | FENM | 10 | — | ↓ | — | — |
| | | 20 | ↓ | ↓ | — | — |
| | | 30 | ↓ | ↓ | — | — |
| Prophylactic | (R,S)- | 30 | ↓ | ↓ | — | — |
| (5 min before | ketamine | | | | | |
| stress) | FENM | 20 | ↑ | — | — | — |
| Antidepressant | (R,S)- | 30 | — | ↓ | — | — |
| (5 min after | ketamine | | | | | |
| stress) | FENM | 10 | — | ↓ | — | — |
| | | 20 | — | ↓ | ↓ | — |
| | | 30 | — | ↓ | ↓ | — |
| Antidepressant | (R,S)- | 30 | — | — | — | — |
| (5 min after | ketamine | | | | | |
| stress; | FENM | 20 | — | ↓ | — | — |
| extinction) | | | | | | |
| Extinction (5 | (R,S)- | 30 | — | — | — | — |
| min before re- | ketamine | | | | | |
| exposure 1) | FENM | 20 | — | — | — | — |
| Extinction (5 | (R,S)- | 30 | — | — | — | — |
| min after re- | ketamine | | | | | |
| exposure 1) | FENM | 20 | ↓ | — | ↓ | — |

We have previously reported that (R,S)-ketamine (30 mg/kg) is effective as a prophylactic against stress when administered 1 week before stress. In these studies, we showed that (R,S)-ketamine is effective against social defeat (SD), learned helplessness (LH), CFC, and chronic corticosterone (CORT) (11-13). Recently, we have found that 5-HT₄R agonists are effective prophylactics, and Gould and colleagues reported that group II metabotropic glutamate receptor (mGlu_{2/3}) antagonists are also protective against stress (39, 40). Although these four newer drugs target distinct receptors from (R,S)-ketamine, to date, we have not reported on other NMDAR antagonists being effective prophylactics. Therefore, it is of interest that FENM, which also targets NMDARs, is effective as a prophylactic.

Consistent with previous studies indicating the importance of NMDARs for the treatment of affective disorders, our data reinforce emerging evidence that NMDARs are also a key target for the prevention of stress-related psychiatric illness (22). NMDAR activity is intricately linked to synaptic plasticity and, accordingly, plays a significant role in fear learning and memory formation (41, 42). Because NMDARs can act as synchrony detectors due to their unique Mg′ blockers, altering baseline NMDAR function via pharmacological agents in unstressed individuals could lead to modulations in neural network function, particularly in brain regions involved in the encoding and recall of fear (43). Ultimately, this modification could prove beneficial in reducing fearful responses and buffering neurobiological responses to stress in the presence of trauma (42). Moreover, because synaptogenesis is critical to the maintenance, rather than induction, of (R,S)-ketamine's antidepressant effects, the effect of NMDAR inhibition on synaptic plasticity could contribute to the long-term enhancement of stress resilience demonstrated by both (R,S)-ketamine and FENM. Further research is necessary to determine whether FENM can also modulate neural network dynamics in a manner similar to (R,S)-ketamine and whether this action directly contributes to 1-BNM's actions as a prophylactic.

When administered 5 min before CFC, (R,S)-ketamine, but not FENM, increased freezing during CFC training and decreased freezing during context re-exposures. The increase in freezing behavior during CFC encoding is most likely due to the anesthetic and psychotropic qualities of (R,S)-ketamine, which were active during the training session. We hypothesize that the subsequent decrease in fear expression and decrease in immobility time in the FST is due to (R,S)-ketamine mice not fully experiencing the stressful CFC procedure, as the saline- and FENM-injected mice did. In previous studies examining prophylactic (R,S)-ketamine, we and others do not find prophylactic efficacy of (R,S)-ketamine if the time interval is altered from the 1-week time period (13, 14).

Interestingly, we found that FENM, administered 5 minutes after stress, was effective at reducing depressive behavior when the FST was administered either 4 or 6 days following the initial stressor. These results differ from our experimental results with (R,S)-ketamine, which indicate antidepressant efficacy of ketamine only when the FST was administered 6 days post-stress. Our data suggest that, although both compounds may act as rapid-acting antidepressants, the effects of (R,S)-ketamine may require more time to manifest in preclinical models compared to FENM. While FENM's acute actions in the brain are still unknown, (R,S)-ketamine increases BDNF signaling via downstream inactivation of eukaryotic elongation factor 2 kinase (eEFK2) which ultimately results in increased BDNF expression (44). Acute BDNF upregulation, while it does not contribute to the sustained actions of (R,S)-ketamine, is known to be necessary for the compound's rapid-acting antidepressant effects (44). Therefore, FENM may upregulate BDNF signaling or expression even faster than (R,S)-ketamine, leading to a more rapid manifestation of its antidepressant actions. Further studies are necessary to examine the acute neurobiological actions of FENM.

Notably, FENM had significant effects in reducing anxiety-like behavior in the OF and MB and suppressing novelty-induced hypophagia. These behavioral results contrast directly with those seen after (R,S)-ketamine administration, which consistently fails to reduce anxiety-like behavior in preclinical rodent models (11-13, 29, 39). Our data indicate that FENM may be efficacious at preventing or treating stress-related anxiety-like behavior, although further study is necessary to determine the biological mechanisms underlying these effects and whether these findings can translate to clinical use.

Overall, the present study has identified a novel NMDAR antagonist that is efficacious at preventing and treating stress-induced fear, depressive-like, and anxiety-like behavior. These data reinforce the NMDAR as a key target for regulating a variety of stress-related behaviors. Future studies may lead to a deeper understanding of how NMDAR antagonists administered before or after stress can modulate a variety of stress-related maladaptive behaviors.

### REFERENCES

1. (2019): Depression Fact Sheet. World Health Organization.
2. (2019): Major Depression Statistics. National Institute of Mental Health.
3. Kornstein S G, Schneider R K (2001): Clinical features of treatment-resistant depression. J Clin Psychiatry. 62 Suppl 16:18-25.
4. Huynh N N, McIntyre R S (2008): What Are the Implications of the STAR*D Trial for Primary Care? A Review and Synthesis. Prim Care Companion J Clin Psychiatry. 10:91-96.
5. Aleksandrova L R, Phillips A G, Wang Y T (2017): Antidepressant effects of ketamine and the roles of AMPA glutamate receptors and other mechanisms beyond NMDA receptor antagonism. J Psychiatry Neurosci. 42:222-229.
6. Berman R M, Cappiello A, Anand A, Oren D A, Heninger G R, Charney D S, et al. (2000): Antidepressant effects of ketamine in depressed patients. Biological psychiatry. 47:351-354.
7. Serafini G, Howland R H, Rovedi F, Girardi P, Amore M (2014): The role of ketamine in treatment-resistant depression: a systematic review. Curr Neuropharmacol. 12:444-461.
8. Murrough J W, Perez A M, Pillemer S, Stern J, Parides M K, aan het Rot M, et al. (2013): Rapid and longer-term antidepressant effects of repeated ketamine infusions in treatment-resistant major depression. Biological psychiatry. 74:250-256.
9. Murrough J W, Perez A M, Mathew S J, Charney D S (2011): A case of sustained remission following an acute course of ketamine in treatment-resistant depression. J Clin Psychiatry. 72:414-415.
10. Zarate C A, Jr., Singh J B, Carlson P J, Brutsche N E, Ameli R, Luckenbaugh D A, et al. (2006): A randomized trial of an N-methyl-D-aspartate antagonist in treatment-resistant major depression. Arch Gen Psychiatry. 63:856-864.
11. Brachman R A, McGowan J C, Perusini J N, Lim S C, Pham T H, Faye C, et al. (2016): Ketamine as a Prophylactic Against Stress-Induced Depressive-like Behavior. Biological psychiatry. 79:776-786.
12. Mastrodonato A, Martinez R, Pavlova I P, LaGamma C T, Brachman R A, Robison A J, et al. (2018): Ventral CA3 Activation Mediates Prophylactic Ketamine Efficacy Against Stress-Induced Depressive-like Behavior. Biological psychiatry. 84:846-856.
13. McGowan J C, LaGamma C T, Lim S C, Tsitsiklis M, Neria Y, Brachman R A, et al. (2017): Prophylactic Ketamine Attenuates Learned Fear. Neuropsychopharmacology: official publication of the American College of Neuropsychopharmacology.
14. Amat J, Dolzani S D, Tilden S, Christianson J P, Kubala K H, Bartholomay K, et al. (2016): Previous Ketamine Produces an Enduring Blockade of Neurochemical and Behavioral Effects of Uncontrollable Stress. The Journal of neuroscience: the official journal of the Society for Neuroscience. 36:153-161.
15. Dolzani S D, Baratta M V, Moss J M, Leslie N L, Tilden S G, Sorensen A T, et al. (2018): Inhibition of a Descending Prefrontal Circuit Prevents Ketamine-Induced Stress Resilience in Females. eNeuro. 5.
16. Zorumski C F, Izumi Y, Mennerick S (2016): Ketamine: NMDA Receptors and Beyond. The Journal of neuroscience: the official journal of the Society for Neuroscience. 36:11158-11164.
17. Abdallah C G, Sanacora G, Duman R S, Krystal J H (2015): Ketamine and Rapid-Acting Antidepressants: A Window into a New Neurobiology for Mood Disorder Therapeutics. Annual Review of Medicine. 66:509-523.
18. Gerhard D M, Wohleb E S, Duman R S (2016): Emerging treatment mechanisms for depression: focus on glutamate and synaptic plasticity. Drug Discovery Today. 21:454-464.
19. Autry A E, Adachi M, Nosyreva E, Na E S, Los M F, Cheng P-f, et al. (2011): NMDA receptor blockade at rest triggers rapid behavioural antidepressant responses. Nature. 475:91-95.
20. Zanos P, Moaddel R, Morris P J, Georgiou P, Fischell J, Elmer G I, et al. (2016): NMDAR inhibition-independent antidepressant actions of ketamine metabolites. Nature. 533:481-486.
21. Kavalali E T, Monteggia L M (2015): How does ketamine elicit a rapid antidepressant response? Current Opinion in Pharmacology. 20:35-39.
22. Moda-Sava R N, Murdock M H, Parekh P K, Fetcho R N, Huang B S, Huynh T N, et al. (2019): Sustained rescue of prefrontal circuit dysfunction by antidepressant-induced spine formation. Science. 364:eaat8078.
23. Hashimoto K (2016): Ketamine's antidepressant action: beyond NMDA receptor inhibition. Expert Opinion on Therapeutic Targets. 20:1389-1392.
24. Zanos P, Gould T D (2018): Mechanisms of ketamine action as an antidepressant. Molecular psychiatry. 23:801-811.
25. Moskal J R, Burgdorf J S, Stanton P K, Kroes R A, Disterhoft J F, Burch R M, et al. (2017): The Development of Rapastinel (Formerly GLYX-13); A Rapid Acting and Long Lasting Antidepressant. Curr Neuropharmacol. 15:47-56.
26. Kato T, Fogaca M V, Deyama S, Li X Y, Fukumoto K, Duman R S (2018): BDNF release and signaling are required for the antidepressant actions of GLYX-13. Molecular psychiatry. 23:2007-2017.
27. Yang B, Ren Q, Ma M, Chen Q X, Hashimoto K (2016): Antidepressant Effects of (+)-MK-801 and (−)-MK-801 in the Social Defeat Stress Model. Int J Neuropsychopharmacol. 19.
28. Yang C, Shirayama Y, Zhang J C, Ren Q, Yao W, Ma M, et al. (2015): R-ketamine: a rapid-onset and sustained antidepressant without psychotomimetic side effects. Translational psychiatry. 5:e632.
29. Chen B K, LaGamma C T, Xu X, Deng S-X, Brachman R A, Suckow R F, et al. (2019): Ovarian hormones mediate the prophylactic efficacy of (R,S)-ketamine and (2R,6R)-hydroxynorketamine in female mice. bioRxiv.712752.
30. Kishi T, Matsunaga S, Iwata N (2017): A Meta-Analysis of Memantine for Depression. J Alzheimers Dis. 57:113-121.
31. Kishi T, Matsunaga S, Oya K, Nomura I, Ikuta T, Iwata N (2017): Memantine for Alzheimer's Disease: An Updated Systematic Review and Meta-analysis. J Alzheimers Dis. 60:401-425.
32. Gideons E S, Kavalali E T, Monteggia L M (2014): Mechanisms underlying differential effectiveness of memantine and ketamine in rapid antidepressant responses. Proceedings of the National Academy of Sciences of the United States of America. 111:8649-8654.
33. Salabert A S, Fonta C, Fontan C, Adel D, Alonso M, Pestourie C, et al. (2015): Radiolabeling of [18F]-fluoroethylnormemantine and initial in vivo evaluation of this innovative PET tracer for imaging the PCP sites of NMDA receptors. Nucl Med Biol. 42:643-653.
34. Salabert A S, Mora-Ramirez E, Beaurain M, Alonso M, Fontan C, Tahar H B, et al. (2018): Evaluation of [(18)F]FNM biodistribution and dosimetry based on whole-body PET imaging of rats. Nucl Med Biol. 59:1-8.
35. Mastrodonato A, Cohensedgh O, LaGamma C T, McGowan J C, Hunsberger H C, Denny C A (2019): Prophylactic (R,S)-ketamine selectively protects against inflammatory stressors. Behav Brain Res. 112238.
36. McGowan J C, Hill C, Mastrodonato A, LaGamma C T, Kitayev A, Brachman R A, et al. (2018): Prophylactic ketamine alters nucleotide and neurotransmitter metabolism in brain and plasma following stress. Neuropsychopharmacology: official publication of the American College of Neuropsychopharmacology. 43:1813-1821.
37. Chen B K, Mendez-David I, Luna V M, Faye C, Gardier A M, David D J, et al. (2020): Prophylactic efficacy of 5-HT(4)R agonists against stress. Neuropsychopharmacology. 45:542-552.
38. Chen B K, LaGamma C T, Xu X, Deng S-X, Brachman R A, Suckow R F, et al. (2019): Ovarian hormones mediate the prophylactic efficacy of (<em>R,S</em>)-ketamine and (2<em>R</em>,6<em>R</em>)-hydroxynorketamine in female mice. bioRxiv.712752.
39. Chen B K, Mendez-David I, Faye C, Gardier A M, David D J, Denny C A (2019): Prophylactic efficacy of 5-HT4R agonists against stress. bioRxiv.712786.
40. Highland J N, Zanos P, Georgiou P, Gould T D (2019): Group II metabotropic glutamate receptor blockade promotes stress resilience in mice. Neuropsychopharmacology: official publication of the American College of Neuropsychopharmacology. 44:1788-1796.
41. Riaza Bermudo-Soriano C, Perez-Rodriguez M M, Vaquero-Lorenzo C, Baca-Garcia E (2012): New perspectives in glutamate and anxiety. Pharmacology Biochemistry and Behavior. 100:752-774.
42. Horn S R, Charney D S, Feder A (2016): Understanding resilience: New approaches for preventing and treating PTSD. Experimental Neurology. 284:119-132.
43. Shin L M, Liberzon I (2010): The neurocircuitry of fear, stress, and anxiety disorders. Neuropsychopharmacology: official publication of the American College of Neuropsychopharmacology. 35:169-191.
44. Björkholm C, Monteggia L M (2016): BDNF—a key transducer of antidepressant effects. Neuropharmacology. 102:72-79.
45. Drew M R, Denny C A, Hen R (2010): Arrest of adult hippocampal neurogenesis in mice impairs single- but not multiple-trial contextual fear conditioning. Behav Neurosci. 124:446-454.
46. Denny C A, Kheirbek M A, Alba E L, Tanaka K F, Brachman R A, Laughman K B, et al. (2014): Hippocampal Memory Traces Are Differentially Modulated by Experience, Time, and Adult Neurogenesis. Neuron. 83:189-201.
47. Brachman R A, McGowan J C, Perusini J N, Lim S C, Pham T H, Faye C, et al. (2016): Ketamine as a Prophylactic Against Stress-Induced Depressive-like Behavior. Biol Psychiatry. 79:776-786.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, and publications are cited throughout this application, the disclosures of which, particularly, including all disclosed chemical structures, are incorporated herein by reference. Citation of the above publications or documents is not intended as an admission that any of the foregoing is pertinent prior art, nor does it constitute any admission as to the contents or date of these publications or documents. All references cited herein are incorporated by reference to the same extent as if each individual publication, patent application, or patent, was specifically and individually indicated to be incorporated by reference.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1. A method for preventing or delaying a stress-induced affective disorder or stress-induced psychopathology in a subject, comprising administering an effective amount of a pharmaceutic composition comprising fluoroethylnormemantine (FENM), or an analog, a pharmaceutically acceptable salt, a derivative, or a metabolite thereof, to the subject at least about 72 hours prior to a stressor.

2. (canceled)

3. The method of claim 1, wherein the pharmaceutic composition is administered to the subject about 72 hours to about 3 weeks prior to a stressor.

4. The method of claim 1, wherein the pharmaceutic composition is administered to the subject about 72 hours to about 2 weeks prior to a stressor.

5. The method of claim 1, wherein the pharmaceutic composition is administered to the subject about 1 week prior to a stressor.

6. The method of claim 1, wherein the pharmaceutic composition is administered to the subject once prior to a stressor.

7. (canceled)

8. (canceled)

9. The method of claim 1, wherein the pharmaceutic composition is administered orally, intravenously, intranasally, or via injection to the subject.

10. The method of claim 1, wherein the stress-induced affective disorder comprises depression and/or fear.

11. The method of claim 1, wherein the stress-induced affective disorder comprises major depressive disorder and/or posttraumatic stress disorder (PTSD).

12. The method of claim 1, wherein the stress-induced affective disorder is selected from the group consisting of: depressive-like behavior and associated affective disorders, anhedonic behavior and associated affective disorders, anxiety and associated affective disorders, cognitive impairments and deficits and associated disorders, and combinations thereof.

13. (canceled)

14. (canceled)

15. The method of claim 1, preventing or delaying stress-induced cognitive impairment and/or decline.

16. The method of claim 1, further comprising administering an effective amount of an anti-depressant, an anxiolytic, or combinations thereof.

17. The method of claim 1, further comprising administering an effective amount of a selective serotonin reuptake inhibitor (SSRI), or a pharmaceutically acceptable salt or derivative thereof.

18. The method of claim 1, further comprising administering an effective amount of fluoxetine, paroxetine, sertraline, lithium, riluzole, prazosin, lamotrigine, ifenprodil, or combinations thereof.

19. The method of claim 1, wherein the subject is a mammal.

20. The method of claim 1, wherein the subject is a human.

21.-23. (canceled)
